(19) United States
(12) Patent Application Publication
(10) Pub. No.: US 2003/0022170 A1
(43) Pub. Date: Jan. 30, 2003

(54) Novel fibroblast growth factors and therapeutic and diagnostic uses therefor

(75) Inventor: Mehran Mohamad Khodadoust, Chestnut Hill, MA (US)

Correspondence Address: FOLEY, HOAG & ELIOT, LLP, PATENT GROUP, ONE POST OFFICE SQUARE, BOSTON, MA, 02109, US

(73) Assignee: Millenium Pharmaceuticals, Inc.

(21) Appl. No.: 09/820,596

(22) Filed: Mar. 29, 2001

Related U.S. Application Data
(63) Continuation of application No. 09/036,594, filed on Mar. 6, 1998.

(51) Int. Cl. C12Q 1/68; C07H 21/04

(52) U.S. Cl. 435/6; 536/24.3; 536/25.32

(57) ABSTRACT

The present invention relates to the discovery of novel genes encoding a fibroblast growth factor, MFGF. Therapeutics, diagnostics and screening assays based on these molecules are also disclosed.

### 1. BACKGROUND OF THE INVENTION

Fibroblast growth factors (FGFs), currently comprising at least twelve members, are polypeptide mitogens. Although referred to as fibroblast growth factors, in fact these molecules trigger a variety of biological responses in many different cell types, including those of mesoderm and neurectoderm origin, such as endothelial cells, smooth muscle cells, adrenal cortex cells, prostatic and retina epithelial cells, oligodendrocytes, astrocytes, chrondocytes, myoblasts, and osteoblasts. These factors have been shown to be involved in a variety of developmental processes, including: angiogenesis, wound healing and tumorigenicity.

Members of this family are likely to be critical regulators of skeletal muscle development in vivo as a number of FGF family members and FGF receptors are: 1) localized to skeletal muscle, 2) present in high levels in diseased and regenerating skeletal muscle, and 3) required for the maintenance of primary mouse and chick skeletal muscle cultures.

FGF-2, FGF4, FGF-5, FGF-6 and FGF-8 mRNA appear to be expressed in skeletal muscle cells as they have been localized to the myotomal muscle region of the somites and in the developing limb muscle masses. Early embryonic hearts express high levels of FGF-1 (formerly known as acidic fibroblast growth factor) and FGF-2 (formerly known as basic fibroblast growth factor) as well as their high affinity receptors (FGFR) types 1, 2 and 3. The expression of both FGFs and FGFRs is developmentally regulated during heart formation. It is thought that FGF signaling may play an important role in establishment of heart size, thickness and shape.

Adult cardiac myocytes lose the capacity to divide. However, they still express multiple growth factors and growth factor receptors. The role of the growth factors and growth factor receptors in the adult heart is unclear. Cummins, et. al. have reported that some changes in gene expression under conditions such as ischemia or volume overload that lead to adult cardiomyocyte hypertrophy are FGF-2 mediated (Cummins et al.(1993) Cardiovasc. Res. 27:1150-11540). Parker and Schneider in in vitro experiments have shown that FGF-2 activates oncogene expression, thereby inducing the synthesis of fetal-like proteins (Parker and Schneider (1991) Annu. Rev. Physiol. 53:179-200). FGF-2 has also been reported to reduce myocardial infarct size following temporary coronary occlusion (Horrigan, et al. (1996) Prog. Growth Factor Res. 5:145-158). The expression of FGF-1 has been shown to be greatly increased in viable cardiomyocytes close to small necrotic myocardial areas. FGF-1, therefore, may play a specific physiological role in a complex cascade leading to collateral growth and remodeling in response to ischemia.

FGF-8, also known as androgen induced growth factor (AIGF), is a fibroblast growth factor which was isolated from an androgen-dependent mouse mammary carcinoma cell line (SC-3). (Tanaka et al., (1992) Proc. Natl. Acad. Sci. USA 89:8928-8932). Studies have shown that androgen-dependent growth is mediated by FGF-8 through an autocrine mechanism.

### 2. SUMMARY OF THE INVENTION

The present invention is based, at least in part, on the discovery of novel human and murine genes encoding novel proteins, which have sequence homologies with known fibroblast growth factors (FGFs). The newly identified proteins and nucleic acids described herein are referred to as “MFGFs” and are exemplified here by both human and murine homologs of this gene. The human MFGF gene (herein referred to as hMFGF) transcript is shown in FIG. 1 (SEQ ID NO. 1) and includes 5′ and 3′ untranslated regions and a 621 base pair open reading frame (SEQ ID NO. 3) encoding a 207 amino acid polypeptide having SEQ ID NO. 2. The mature protein, i.e., the full length protein without the signal sequence is comprised of about 179 amino acids. Human MFGF is expressed predominantly in heart tissue. A nucleic acid comprising the cDNA encoding the full length human MFGF polypeptide was deposited at the American Type Culture Collection (12301 Parklawn Drive, Rockville, Md.) on Jan. 8, 1998 and has been assigned ATCC Designation No. 209574. The murine homolog of hMFGF has also been isolated and is herein referred to as mMFGF. The mMFGF gene transcript is shown in FIG. 2 (SEQ ID NO. 4) and includes 5′ and 3′ untranslated regions and a 621 bas pair open reading frame (SEQ ID NO. 6) encoding a 207 amino acid polypeptide having SEQ ID NO. 5. The mature protein, i.e., the full length protein without the signal sequence is, like the human MFGF homolog, comprised of about 179 amino acids. A nucleic acid comprising the cDNA encoding the full length murine MFGF polypeptide was deposited at the American Type Culture Collection (12301 Parklawn Drive, Rockville, Md.) on Feb. 26, 1998 and has been assigned ATCC Designation No. 209648.

An amino acid and nucleotide sequence analysis using the BLAST program (Altschul et al. (1990) J. Mol. Biol. 215:403) revealed that certain portions of the amino acid and nucleic acid sequences of the newly identified human and murine MFGF proteins and nucleic acids have a sequence similarity with certain regions of other fibroblast growth factors. In particular, MFGF contains a conserved region of basic residues believed to be involved in binding to heparin sulfate proteoglycans present on the cell surface and in the extracellular matrix (amino acid residues 154 to 164 of SEQ ID NO. 2 for hMFGF and amino acid residues 154 to 164 of SEQ ID NO. 5 for mMFGF). MFGF further comprises a FGFR binding domain which, by analogy with FGF-2 (bFGF) includes amino acid residues 33 to 38 and 152-161 of both SEQ ID NO. 2 (hMFGF) and SEQ ID NO. 5 (mMFGF). Significantly, the predicted mature processed forms of human and murine MFGF contain a pair of cysteine residues, which although characteristic of the FGF family members in general, are uniquely spaced in MFGF, FGF-8, and a third related growth factor, human “FGF-13” (which is the FGF described in WO 96/39508, and which, we note here, is unrelated to mu FGF-13). Thus MFGF, together with FGF-8 and the human “FGF-13”, define a new evolutionary subfamily of fibroblast growth factors. The amino-terminal region (amino acid residues 1 to 28 of SEQ ID NO. 2 for hNEGF and amino acid residues 1 to 28 of of SEQ ID NO. 5 for nMFGF) represent a potential signal peptide which could be processed away during cotranslational import into the endoplasmic reticulum. Thus, MFGF is believed to share at least some of the biological activities of known fibroblast growth factors, in particular the FGF signaling activities. Although MFGF is apparently most highly related to FGF-8 and “FGF-13”, except for the presence of other small regions of homology between MFGF and known FGF proteins, the other portions of MFGF are significantly different.

vIn one aspect, the invention features isolated MFGF nucleic acid molecules. In one embodiment, the MFGF nucleic acid is from a vertebrate. In a preferred embodiment, the MFGF nucleic acid is from a mammal, e.g. a human. In an even more preferred embodiment, the nucleic acid has the nucleic acid sequence set forth in SEQ ID NO. 1, 3, 4, or 6 or a portion thereof In another embodiment of the invention, the nucleic acid is murine in origin and has the nucleic acid sequence set forth in SEQ ID NO. 4 and/or 6 or a portion thereof The disclosed molecules can be non-coding, (e.g. a probe, antisense, or ribozyme molecule) or can encode a functional MFGF polypeptide (e.g. a polypeptide which specifically modulates biological activity, by acting as either an agonist or antagonist of at least one bioactivity of the human MFGF polypeptide). In one embodiment, the nucleic acid molecule can hybridize to the MFGF gene contained in ATCC designation number 209574 (hMFGF) or 209648 (mMFGF). In another embodiment, the nucleic acid of the present invention can hybridize to a vertebrate MFGF gene or to the complement of a vertebrate MFGF gene. In a further embodiment, the claimed nucleic acid can hybridize with a nucleic acid sequence shown in FIG. 1 (SEQ ID NOS. 1 and 3) or a complement thereof. In another embodiment, the claimed nucleic acid can hybridize with a nucleic acid sequence shown in FIG. 2 (SEQ ID NOS. 4 and 6) or a complement thereof. In a preferred embodiment, the hybridization is conducted under mildly stringent or stringent conditions.

In further embodiments, the nucleic acid molecule is an MFGF nucleic acid that is at least about 70%, preferably about 80%, more preferably about 85%, and even more preferably at least about 90% or 95% homologous to the nucleic acid shown as SEQ ID NOS: 1, 3, 4, or 6 or to the complement of the nucleic acid shown as SEQ ID NOS: 1, 3, 4, or 6. In a further embodiment, the nucleic acid molecule is an MFGF nucleic acid that is at least about 70%, preferably at least about 80%, more preferably at least about 85% and even more preferably at least about 90% or 95% similar in sequence to the MFGF nucleic acid contained in ATCC designation number 209574 or ATCC designation number 209648.

The invention also provides probes and primers comprising substantially purified oligonucleotides, which correspond to a region of nucleotide sequence which hybridizes to at least about 6, at least about 10, at least about 15, at least about 20, or preferably at least about 25 consecutive nucleotides of the sequence set forth as SEQ ID NO. 1 or SEQ ID NO. 4 or complements of the sequence set forth as SEQ ID NOS. 1 or 4 or naturally occurring mutants or allelic variants thereof In preferred embodiments, the probe/primer further includes a label group attached thereto, which is capable of being detected.

For expression, the subject nucleic acids can be operably linked to a transcriptional regulatory sequence, e.g., at least one of a transcriptional promoter (e.g., for constitutive expression or inducible expression) or transcriptional enhancer sequence. Such regulatory sequences in conjunction with an MFGF nucleic acid molecule can provide a useful vector for gene expression. This invention also describes host cells transfected with said expression vector whether prokaryotic or eukaryotic and in vitro (e.g. cell culture) and in vivo (e.g. transgenic) methods for producing MFGF proteins by employing said expression vectors.

In another aspect, the invention features isolated MFGF polypeptides, preferably substantially pure preparations, e.g. of plasma purified or recombinantly produced polypeptides. The MFGF polypeptide can comprise a full length protein or can comprise smaller fragments corresponding to one or more particular motifs/domains, or fragments comprising at least about 6, 10, 25, 50, 75, 100, 125, 130, 135, 140, 145, 150, 155, 160, 165, 170, 175, 180, 185, 190, 195, or 200 amino acids in length. In particularly preferred embodiments, the subject polypeptide has an MFGF bioactivity, for example, it is capable of binding to and/or otherwise altering the activity of a receptor, particularly an FGF receptor (FGFR) family type.

In a preferred embodiment, the polypeptide is encoded by a nucleic acid which hybridizes with the nucleic acid sequence represented in SEQ ID NOS. 1, 3, 4, or 6. In a further preferred embodiment, the MFGF polypeptide is comprised of the amino acid sequence set forth in SEQ ID NO. 2 or SEQ ID NO. 5. The subject MFGF protein also includes within its scope modified proteins, e.g. proteins which are resistant to post-tanslational modification, for example, due to mutations which alter modification sites (such as tyrosine, threonine, serine or aspargine residues), or which prevent glycosylation of the protein, or which prevent interaction of the protein with intracellular proteins involved in signal transduction.

The MFGF polypeptides of the present invention can be glycosylated, or conversely, by choice of the expression system or by modification of the protein sequence to preclude glycosylation, reduced carbohydrate analogs can also be provided. Glycosylated forms can be obtained based on derivatization with glycosaminoglycan chains. Also, MFGF polypeptides can be generated which lack an endogenous signal sequence (though this is typically cleaved off even if present in the pro-form of the protein).

In yet another preferred embodiment, the invention features a purified or recombinant polypeptide, which has the ability to modulate, e.g., mimic or antagonize, an activity of a wild-type MFGF protein. Preferably, the polypeptide comprises an amino acid sequence identical or homologous to a sequence designated in SEQ ID NO. 2 or SEQ ID NO. 5.

Another aspect of the invention features chimeric molecules (e.g., fusion proteins) comprising an MFGF protein. For instance, the MFGF protein can be provided as a recombinant fusion protein which includes a second polypeptide portion, e.g., a second polypeptide having an amino acid sequence unrelated (heterologous) to the MFGF polypeptide. A preferred MFGF fusion protein is an immunoglobulin-MFGF fusion protein, in which an immunoglobulin constant region is fused to an MFGF polypeptide.

Yet another aspect of the present invention concerns an immunogen comprising an MFGF polypeptide in an immunogenic preparation, the immunogen being capable of eliciting an immune response specific for an MFGF polypeptide; e.g. a humoral response, an antibody response and/or cellular response. In a preferred embodiment, the immunogen comprises an antigenic determinant, e.g. a unique determinant of a protein encoded by the nucleic acid set forth in SEQ ID NO. 1, 3, 4, or 6; or as set forth in SEQ ID NOS. 2 or 5.

A still further aspect of the present invention features antibodies and antibody preparations specifically reactive with an epitope of an MFGF protein.

The invention also features transgenic non-human animals which include (and preferably express) a heterologous form of an MFGF gene described herein, or which misexpress an endogenous MFGF gene (e.g., an animal in which expression of one or more of the subject MFGF proteins is disrupted). Such transgenic animals can serve as animal models for studying cellular and/or tissue disorders comprising mutated or mis-expressed MFGF alleles or for use in drug screening. Alternatively, such transgenic animals can be useful for expressing recombinant MFGF polypeptides.

The invention further features assays and kits for determining whether an individual's MFGF genes and/or proteins are defective or deficient (e.g in activity and/or level), and/or for determining the identity of MFGF alleles. In one embodiment, the method comprises the step of determining the level of MFGF protein, the level of MFGF mRNA and/or the transcription rate of an MFGF gene. In another preferred embodiment, the method comprises detecting, in a tissue of the subject, the presence or absence of a genetic alteration, which is characterized by at least one of the following: a deletion of one or more nucleotides from a gene; an addition of one or more nucleotides to the gene; a substitution of one or more nucleotides of the gene; a gross chromosomal rearrangement of the gene; an alteration in the level of a messenger RNA transcript of the gene; the presence of a non-wild type splicing pattern of a messenger RNA transcript of the gene; and/or a non-wild type level of the MFGF protein.

For example, detecting a genetic alteration or the presence of a specific polymorphic region can include (i) providing a probe/primer comprised of an oligonucleotide which hybridizes to a sense or antisense sequence of an MFGF gene or naturally occurring mutants thereof, or 5′ or 3′ flanking sequences naturally associated with the MFGF gene; (ii) contacting the probe/primer with an appropriate nucleic acid containing sample; and (iii) detecting, by hybridization of the probe/primer to the nucleic acid, the presence or absence of the genetic alteration. Particularly preferred embodiments comprise: 1) sequencing at least a portion of an MFGF gene, 2) performing a single strand conformation polymorphism (SSCP) analysis to detect differences in electrophoretic mobility between mutant and wild-type nucleic acids; and 3) detecting or quantitating the level of an MFGF protein in an immunoassay using an antibody which is specifically immunoreactive with a wild-type or mutated MFGF protein.

Information obtained using the diagnostic assays described herein (alone or in conjunction with information on another genetic defect, which contributes to the same disease) is useful for diagnosing or confirming that a symptomatic subject has a genetic defect (e.g. in an MFGF gene or in a gene that regulates the expression of an MFGF gene), which causes or contributes to the particular disease or disorder. Alternatively, the information (alone or in conjunction with information on another genetic defect, which contributes to the same disease) can be used prognostically for predicting whether a non-symptomatic subject is likely to develop a disease or condition, which is caused by or contributed to by an abnormal MFGF activity or protein level in a subject. In particular, the assays permit one to ascertain an individual's predilection to develop a condition associated with a mutation in MFGF, where the mutation is a single nucleotide polymorphism (SNP). Based on the prognostic information, a doctor can recommend a regimen (e.g. diet or exercise) or therapeutic protocol useful for preventing or prolonging onset of the particular disease or condition in the individual.

In addition, knowledge of the particular alteration or alterations, resulting in defective or deficient MFGF genes or proteins in an individual, alone or in conjunction with information on other genetic defects contributing to the same disease (the genetic profile of the particular disease) allows customization of therapy for a particular disease to the individual's genetic profile, the goal of pharmacogenomics. For example, an individual's MFGF genetic profile or the genetic profile of a disease or condition to which MFGF genetic alterations cause or contribute, can enable a doctor to: 1) more effectively prescribe a drug that will address the molecular basis of the disease or condition; and 2) better determine the appropriate dosage of a particular drug. For example, the expression level of MFGF proteins, alone or in conjunction with the expression level of other genes known to contribute to the same disease, can be measured in many patients at various stages of the disease to generate a transcriptional or expression profile of the disease. Expression patterns of individual patients can then be compared to the expression profile of the disease to determine the appropriate drug and dose to administer to the patient.

The ability to target populations expected to show the highest clinical benefit, based on the MFGF or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling (e.g. since the use of MFGF as a marker is useful for optimizing effective dose).

In another aspect, the invention provides methods for identifying a compound which modulates an MFGF activity, e.g. the interaction between an MFGF polypeptide and a target peptide In a preferred embodiment, the method includes the steps of (a) forming a reaction mixture including: (i) an MFGF polypeptide, (ii) an MFGF binding partner (e.g., an MFGF receptor or a heparin sulfate proteoglycan), and (iii) a test compound; and (b) detecting interaction of the MFGF polypeptide and the MFGF binding protein. A statistically significant change (potentiation or inhibition) in the interaction of the MFGF polypeptide and MFGF binding protein in the presence of the test compound, relative to the interaction in the absence of the test compound, indicates a potential agonist (mimetic or potentiator) or antagonist (inhibitor) of MFGF bioactivity for the test compound. The reaction mixture can be a cell-free protein preparation, e.g., a reconstituted protein mixture or a cell lysate, or it can be a recombinant cell including a heterologous nucleic acid recombinantly expressing the MFGF binding partner.

In preferred embodiments, the step of detecting interaction of the MFGF and MFGF binding partner is a competitive binding assay. In other preferred embodiments, at least one of the MFGF polypeptide and the MFGF binding partner comprises a detectable label, and interaction of the MFGF and MFGF binding partner is quantified by detecting the label in the complex. The detectable label can be, e.g., a radioisotope, a fluorescent compound, an enzyme, or an enzyme co-factor. In other embodiments, the complex is detected by an immunoassay.

Yet another exemplary embodiment provides an assay for screening test compounds to identify agents which modulate the amount of MFGF produced by a cell. In one embodiment, the screening assay comprises contacting a cell transfected with a reporter gene operably linked to an MFGF promoter with a test compound and determining the level of expression of the reporter gene. The reporter gene can encode, e.g., a gene product that gives rise to a detectable signal such as: color, fluorescence, luminescence, cell viability, relief of a cell nutritional requirement, cell growth, and drug resistance. For example, the reporter gene can encode a gene product selected from the group consisting of chloramphenicol acetyl transferase, luciferase, beta-galactosidase and alkaline phosphatase.

Also within the scope of the invention are methods for treating diseases or disorders which are associated with an aberrant MFGF level or activity or which can benefit from modulation of the activity or level of MFGF. The methods comprise administering, e.g., either locally or systemically to a subject, a pharmaceutically effective amount of a composition comprising an MFGF therapeutic. Depending on the condition being treated, the therapeutic can be an MFGF agonist or an MFGF antagonist.

Other features and advantages of the invention will be apparent from the following detailed description and claims.

### 3. BRIEF DESCRIPTION OF THE FIGURES

FIG. 1 shows the nucleotide sequence of a full length cDNA encoding human MFGF including 5′ and 3′ untranslated regions and coding sequences (SEQ ID NO. 1) and the deduced amino acid sequence of the MFGF protein (SEQ ID NO. 2).

FIG. 2 shows the nucleotide sequence of a full length cDNA encoding murine MFGF including 5′ and 3′ untranslated regions and coding sequences (SEQ ID NO. 4) and the deduced amino acid sequence of the murine MFGF protein (SEQ ID NO. 5).

FIG. 3 shows an alignment of the amino acid sequence of human MFGF having SEQ ID NO. 2 and the amino acid sequence of murine MFGF having SEQ ID NO. 5 with human FGF-1 (SEQ ID NO. 7; GenBank Accession No. E03692), human FGF-2 (SEQ ID NO. 8; GenBank Accession No. E05628), mouse FGF-3 (INT-2) (SEQ ID NO. 9; GenBank Accession No. X68450), mouse FGF-13 (SEQ ID NO. 10; GenBank Accession No. AF020737), and human FGF-8 (SEQ ID NO. 11; GenBank Accession No. U36223). The uniquely spaced conserved cysteine residues which are characteristic of MFGF, FGF-8 and “FGF-13” are circled. Note the position of the more carboxy-termine cysteine residue is conserved in all of the FHF family members. The FGFR binding regions of human FGF-2 human, FGF8, hMFGF and MFGF (i) and (ii) are boxed.

### 4. DETAILED DESCRIPTION OF THE INVENTION

4.1. General

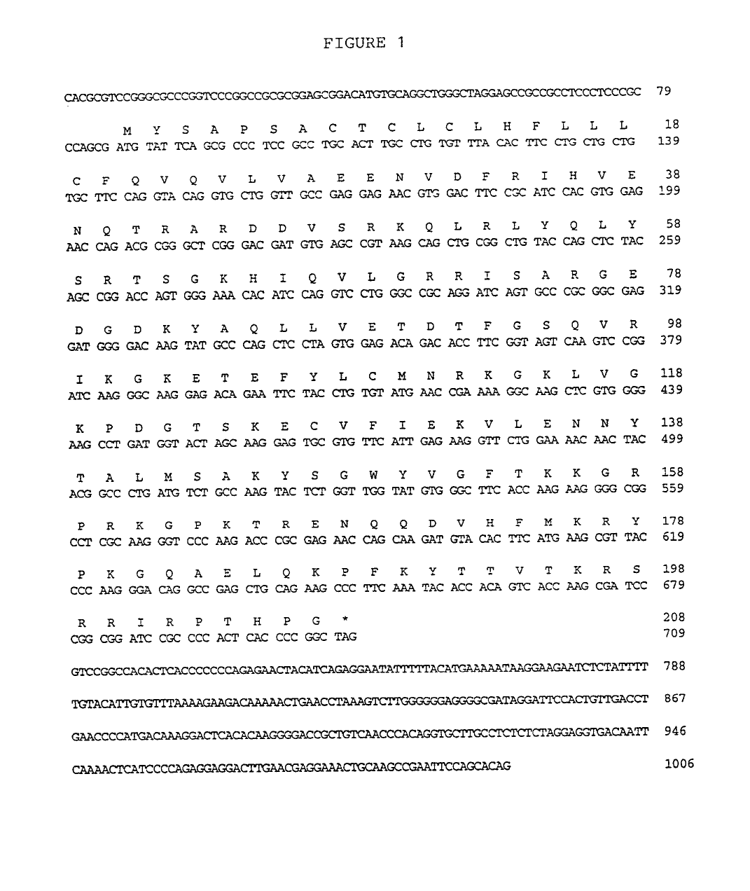

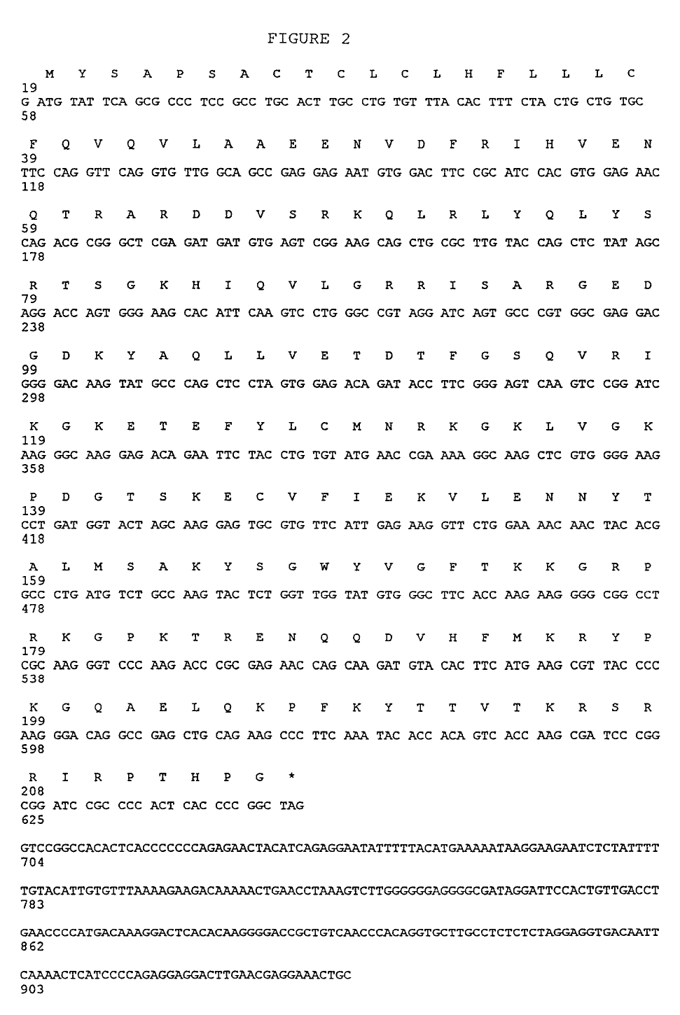

The invention is based at least in part on the discovery of a gene encoding a protein having regions which are significantly homologous to regions of known fibroblast growth factors (FGFs). Thus, the genes and proteins disclosed herein are referred to as MFGF genes and proteins. The sequence of the cDNA encoding full length MFGF was determined from a clone obtained from a cDNA library prepared from mRNA of a human heart obtained from a subject who had congestive heart failure. The cDNA encoding the full length human MFGF protein and comprising 5′ and 3′ untranslated regions is 1006 nucleotides long and has the nucleotide sequence shown in FIG. 1 and is set forth as SEQ ID NO. 1. The full length human MFGF protein is 207 amino acids long and has the amino acid sequence shown in FIG. 1 and set forth in SEQ ID NO. 2. The coding portion (open reading frame) of SEQ ID No. 1 is set forth as SEQ ID No. 3 and corresponds to nucleotides 86 to 709 of SEQ ID NO. 1. The cDNA encoding the full length human MFGF protein was deposited at the American Type Culture Collection (12301 Parklawn Drive, Rockville, Md.) on Jan. 8, 1998 and has been assigned ATCC Designation No. 209574. The murine MFGF homolog was obtained from cDNA prepared from mouse heart mRNA. The cDNA encoding the full length murine MFGF protein and comprising 5′ and 3′ untranslated regions is 903 nucleotides long and has the nucleotide sequence shown in FIG. 2 and is set forth as SEQ ID NO. 4. The full length murine MFGF protein is 207 amino acids long and has the amino acid sequence shown in FIG. 2 and set forth in SEQ ID NO. 5. The coding portion (open reading frame) of SEQ ID No. 4 is set forth as SEQ ID No. 6 and corresponds to nucleotides 2 to 625 of SEQ ID NO. 4. The cDNA encoding the full length murine MFGF protein was deposited at the American Type Culture Collection (12301 Parklawn Drive, Rockville, Md.) on Feb. 26, 1998 and has been assigned ATCC Designation No. 209648.

Both the human and mouse MFGF proteins comprise a signal peptide from amino acid 1 to amino acid 28 of SEQ ID NO. 2 or 5. This signal sequence is encoded by human MFGF nucleotides 86 to 169 of SEQ ID NO. 1 and by murine MFGF nucleotides 2 to 169 of SEQ ID NO. 4. Thus, the mature MFGF protein has 179 amino acids and comprises the amino acid sequence from about amino acid 29 to amino acid 207 of SEQ ID NO. 2 or SEQ ID NO. 5.

MFGF protein further comprises several functional domains. A prosite pattern search indicated an N-glycosylation site within the NQTR amino acid sequence from amino acid 39 to amino acid 42 of SEQ ID NO. 2 or SEQ ID NO. 5, which is encoded by nucleotides 200 to 211 of SEQ ID NO. 1 and by nucleotides 116 to 127 of SEQ ID NO. 4 respectively. A second N-glycosylation is predicted with the the NYTA amino acid sequence from amino acid 137 to 140 of human (SEQ ID NO. 2) or murine (SEQ ID NO. 5) MFGF protein, which is encoded by nucleotides 494 to 505 of SEQ ID NO. 1 and by nucleotides 410 to 421 of SEQ ID NO. 4 respectively.

As described further in the exemplifications, human multiple tissue Northern blot analysis indicated that MFGF mRNA is expressed predominantly in heart tissue. In particular, there was a preponderance of MFGF message in cardiac muscle as compared to such organs and tissues as prostate, pancreas, kidney, liver, lung, placenta, and brain, as well as other sources of non-cardiac muscle tissue such as skeletal muscle, uterus, colon, small intestine, bladder, and stomach. Furthermore, the murine MFGF cDNA clone was obtained from mouse heart tissue, providing additional support for a conserved function for MFGF in cardiac muscle.

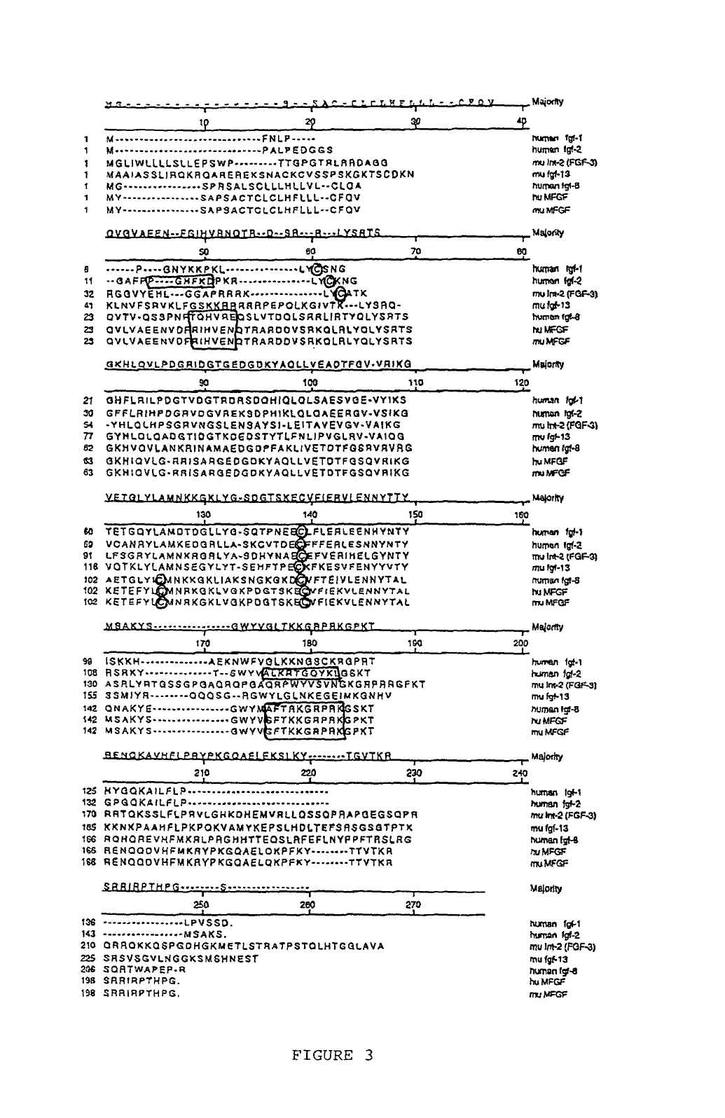

A BLAST search (Altschul et al. (1990) J. Mol. Biol. 215:403) of the nucleic acid and the amino acid sequences of human MFGF revealed that certain portions of the MFGF protein and nucleic acid sequence show homology to certain regions of previously identified fibroblast growth factors (See also, FIG. 3).

However, the overall similarity of MFGF with other FGF nucleic acids and proteins is relatively weak. In fact, the overall percent identity and similarity between human MFGF and human “FGF-13” (described in WO 96/39508 and which is the FGF protein with which MFGF has the highest overall similarity) is about 60% and 82% respectively. At the nucleotide level, human MFGF and human “FGF-13” have about 63% identity. In the case of the next most related protein sequence, human FGF-8, the overall percent identity and similarity with human MFGF is about 60% and 75% respectively. See FIG. 2. At the nucleotide level, human MFGF and human FGF-8 have about 68% identity.

4.2 Definitions

For convenience, the meaning of certain terms and phrases employed in the specification, examples, and appended claims are provided below.

The term “agonist”, as used herein, is meant to refer to an agent that mimics or upregulates (e.g. potentiates or supplements) an MFGF bioactivity. An MFGF agonist can be a wild-type MFGF protein or derivative thereof having at least one bioactivity of the wild-type MFGF, e.g. FGF receptor binding activity. An MFGF therapeutic can also be a compound that upregulates expression of an MFGF gene or which increases at least one bioactivity of an MFGF protein. An agonist can also be a compound which increases the interaction of an MFGF polypeptide with another molecule, e.g, a FGF receptor.

“Antagonist” as used herein is meant to refer to an agent that downregulates (e.g. suppresses or inhibits) at least one MFGF bioactivity. An MFGF antagonist can be a compound which inhibits or decreases the interaction between an MFGF protein and another molecule, e.g., a receptor, such as a FGFR. Accordingly, a preferred antagonist is a compound which inhibits or decreases binding to a FGFR and thereby blocks subsequent activation of the FGFR. An antagonist can also be a compound that downregulates expression of an MFGF gene or which reduces the amount of MFGF protein present. The MFGF antagonist can be a dominant negative form of an MFGF polypeptide, e.g., a form of an MFGF polypeptide which is capable of interacting with a target peptide, e.g., a fibroblast growth factor receptor, but which is not capable of simultaneous binding to heparin sulfate proteoglycans which promote the activation of MFGFRs through an allosteric change in the MFGFR binding domain of MFGF. The MFGF antagonist can also be a nucleic acid encoding a dominant negative form of an MFGF polypeptide, an MFGF antisense nucleic acid, or a ribozyme capable of interacting specifically with an MFGF RNA. Yet other MFGF antagonists are molecules which bind to an MFGF polypeptide and inhibit its action. Such molecules include peptides, e.g., forms of MFGF target peptides which do not have biological activity, and which inhibit binding by MFGF to MFGFRs. Thus, such peptides will bind the active site of MFGF and prevent it from interacting with target peptides, e.g., MFGFR. Yet other MFGF antagonists include antibodies interacting specifically with an epitope of an MFGF molecule, such that binding interferes with hydrolysis. In yet another preferred embodiment, the MFGF antagonist is a small molecule, such as a molecule capable of inhibiting the interaction between an MFGF polypeptide and a target MFGFR. Alternatively, the small molecule can be antagonist by interacting with sites other than the MFGFR binding site, such as the heparin sulfate proteoglycan binding site and inhibit the activity of MFGF by, e.g., altering the tertiary or quaternary structure of the growth factor.

The term “allele”, which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions, and insertions of nucleotides. An allele of a gene can also be a form of a gene containing a mutation. The term “allelic variant of a polymorphic region of an MFGF gene” refers to a region of an MFGF gene having one or several nucleotide sequences found in that region of the gene in other individuals.

“Biological activity” or “bioactivity” or “activity” or “biological function”, which are used interchangeably, for the purposes herein means an effector or antigenic function that is directly or indirectly performed by an MFGF polypeptide (whether in its native or denatured conformation), or by any subsequence thereof. Biological activities include binding to a target peptide, e.g., an FGF receptor or heparin sulfate. An MFGF bioactivity can be modulated by directly affecting an MFGF polypeptide. Alternatively, an MFGF bioactivity can be modulated by modulating the level of an MFGF polypeptide, such as by modulating expression of an MFGF gene.

As used herein the term “bioactive fragment of an MFGF polypeptide” refers to a fragment of a full-length MFGF polypeptide, wherein the fragment specifically mimics or antagonizes the activity of a wild-type MFGF polypeptide. The bioactive fragment preferably is a fragment capable of interacting with a FGF receptor or heparin sulfate.

The term “an aberrant activity”, as applied to an activity of a polypeptide such as MFGF, refers to an activity which differs from the activity of the wild-type or native polypeptide or which differs from the activity of the polypeptide in a healthy subject. An activity of a polypeptide can be aberrant because it is stronger than the activity of its native counterpart. Alternatively, an activity can be aberrant because it is weaker or absent relative to the activity of its native counterpart. An aberrant activity can also be a change in an activity. For example an aberrant polypeptide can interact with a different target peptide. A cell can have an aberrant MFGF activity due to overexpression or underexpression of the gene encoding MFGF.

“Cells”, “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A “chimeric polypeptide” or “fusion polypeptide” is a fusion of a first amino acid sequence encoding one of the subject MFGF polypeptides with a second amino acid sequence defining a domain (e.g. polypeptide portion) foreign to and not substantially homologous with any domain of an MFGF polypeptide. A chimeric polypeptide may present a foreign domain which is found (albeit in a different polypeptide) in an organism which also expresses the first polypeptide, or it may be an “interspecies”, “intergenic”, etc. fusion of polypeptide structures expressed by different kinds of organisms. In general, a fusion polypeptide can be represented by the general formula X-MFGF-Y, wherein MFGF represents a portion of the polypeptide which is derived from an MFGF polypeptide, and X and Y are independently absent or represent amino acid sequences which are not related to an MFGF sequence in an organism including naturally occurring mutants.

The term “nucleotide sequence complementary to the nucleotide sequence set forth in SEQ ID NO. x” refers to the nucleotide sequence of the complementary strand of a nucleic acid strand having SEQ ID NO. x. The term “complementary strand” is used herein interchangeably with the term “complement”. The complement of a nucleic acid strand can be the complement of a coding strand or the complement of a non-coding strand. When referring to double stranded nucleic acids, the complement of a nucleic acid having SEQ ID NO. x refers to the complementary strand of the strand having SEQ ID NO. x or to any nucleic acid having the nucleotide sequence of the complementary strand of SEQ ID NO. x. When referring to a single stranded nucleic acid having the nucleotide sequence SEQ ID NO. x, the complement of this nucleic acid is a nucleic acid having a nucleotide sequence which is complementary to that of SEQ ID NO. x. The nucleotide sequences and complementary sequences thereof are always given in the 5′ to 3′ direction.

A “delivery complex” shall mean a targeting means (e.g. a molecule that results in higher affinity binding of a gene, protein, polypeptide or peptide to a target cell surface and/or increased cellular or nuclear uptake by a target cell). Examples of targeting means include: sterols (e.g. cholesterol), lipids (e.g. a cationic lipid, virosome or liposome), viruses (e.g. adenovirus, adeno-associated virus, and retrovirus) or target cell specific binding agents (e.g. ligands recognized by target cell specific receptors). Preferred complexes are sufficiently stable in vivo to prevent significant uncoupling prior to internalization by the target cell. However, the complex is cleavable under appropriate conditions within the cell so that the gene, protein, polypeptide or peptide is released in a functional form.

As is well known, genes may exist in single or multiple copies within the genome of an individual. Such duplicate genes may be identical or may have certain modifications, including nucleotide substitutions, additions or deletions, which all still code for polypeptides having substantially the same activity. The term “DNA sequence encoding an MFGF polypeptide” may thus refer to one or more genes within a particular individual. Moreover, certain differences in nucleotide sequences may exist between individual organisms, which are called alleles. Such allelic differences may or may not result in differences in amino acid sequence of the encoded polypeptide yet still encode a polypeptide with the same biological activity.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. A degree of homology or similarity of amino acid sequences is a function of the number of amino acids, i.e. structurally related, at positions shared by the amino acid sequences. An “unrelated” or “non-homologous” sequence shares less than 40% identity, though preferably less than 25% identity, with one of the MFGF sequences of the present invention.

The term “interact” as used herein is meant to include detectable relationships or association (e.g. biochemical interactions) between molecules, such as interaction between protein-protein, protein-nucleic acid, nucleic acid-nucleic acid, and protein-small molecule or nucleic acid-small molecule in nature.

The term “isolated” as used herein with respect to nucleic acids, such as DNA or RNA, refers to molecules separated from other DNAs, or RNAs, respectively, that are present in the natural source of the macromolecule. For example, an isolated nucleic acid encoding one of the subject MFGF polypeptides preferably includes no more than 10 kilobases (kb) of nucleic acid sequence which naturally immediately flanks the MFGF gene in genomic DNA, more preferably no more than 5 kb of such naturally occurring flanking sequences, and most preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

The term “MFGF nucleic acid” refers to a nucleic acid encoding an MFGF protein, such as nucleic acids having SEQ ID NO. 1, 3, 4 or 6, fragments thereof, a complement thereof, and derivatives thereof.

The terms “MFGF polypeptide” and “MFGF protein” are intended to encompass polypeptides comprising the amino acid sequence shown as SEQ ID NO. 2 or SEQ ID NO. 5 or fragments thereof, and homologs thereof and include agonist and antagonist polypeptides.

The term “MFGF receptor” or “MFGFR” refers to various cell membrane bound protein receptors capable of binding to and/or transducing a signal from MFGF. The term “FGF receptor” or “FGFR” refers to various cell membrane bound protein receptors capable of binding to and/or transducing a signal from any or all members of the FGF family, e.g. FGFR-2 (Miki et al. (1991) Science 251:72-5), FGFR-3 (Keegan et al. (1991) Ann. N. Y. Acad. Sci. 638:400-2), FGFR4 (Partanen et al. (1991) EMBO J. 10:1347-54) (reviewed in Johnson and Williams (1993) Adv. Cancer Res. 60:1-41, The term “FGFR” also refers to different isoforms of the FGF receptor proteins which may arise by differential splicing mechanisms from a common FGFR gene. Such splicing variants may possess identical or altered ligand binding specificity as in the case of FGFR-2, in which one isoform, arising from a differential splicing event affecting the ligand binding domain, has a dramatically increased affinity for FGF-7 (Slavin (1995) Cell Biology International 19:431-44). In another example, there are unique splice variants of FGFR-3 which bind only FGF-1 (Chellaiah et al. (1994) J. Biol. Chem. 269:11620-7). Furthermore the term “FGFR” as used here is meant to include both the high affinity receptors discussed above, and the low affinity receptors which include a group of cell surface heparan sulfate proteoglycans known as the syndecans (including Syndecan 1, 2, 3 or 4) (Kiefer (1990) Proc. Natl. Acad. Sci. U.S.A. 87:6985-9; Bernfield and Sanderson (1990) Phil. Trans. R. Soc. Lond. 327: 171-86). Studies suggest that the low affinity receptor is an accessory molecule required for binding of FGF to the high affinity receptor. Finally, the term “FGFR” is also meant to refer to a unique cysteine-rich FGF receptor (CFR) (Burrus, et al. (1992) Mol. Cell Biol. 12:5600-9).

The term “heparin sulfate” as used herein is meant to refer to any of a number of chemically related sulfated mucopolysaccharides or mucopoysaccharide sulfuric acid esters. The term “heparin sulfate” as used herein is also meant to connote members of a large family of cell surface heparan sulfate proteoglycans. Both free heparin sulfate and the cell surface heparan sulfate proteoglycans are capable of serving the related function of facilitating FGF binding to any of a number of high-affinity FGFRs as defined above.

The term “MFGF therapeutic” refers to various forms of MFGF polypeptides, as well as peptidomimetics, nucleic acids, or small molecules, which can modulate at least one activity of an MFGF polypeptide, e.g., interaction with an FGF receptor interaction with and/or heparin sulfate, by mimickiing or potentiating (agonizing) or inhibiting (antagonizing) the effects of a naturally-occurring MFGF polypeptide. An MFGF therapeutic which mimics or potentiates the activity of a wild-type MFGF polypeptide is a “MFGF agonist”. Conversely, an MFGF therapeutic which inhibits the activity of a wild-type MFGF polypeptide is a “MFGF antagonist”.

The term “modulation” as used herein refers to both upregulation (i.e., activation or stimulation (e.g., by agonizing or potentiating)) and downregulation (i.e. inhibition or suppression (e.g., by antagonizing, decreasing or inhibiting)).

The term “mutated gene” refers to an allelic form of a gene, which is capable of altering the phenotype of a subject having the mutated gene relative to a subject which does not have the mutated gene. If a subject must be homozygous for this mutation to have an altered phenotype, the mutation is said to be recessive. If one copy of the mutated gene is sufficient to alter the genotype of the subject, the mutation is said to be dominant. If a subject has one copy of the mutated gene and has a phenotype that is intermediate between that of a homozygous and that of a heterozygous subject (for that gene), the mutation is said to be co-dominant.

The “non-human animals” of the invention include mammalians such as rodents, non-human primates, sheep, dog, cow, chickens, amphibians, reptiles, etc. Preferred non-human animals are selected from the rodent family including rat and mouse, most preferably mouse, though transgenic amphibians, such as members of the Xenopus genus, and transgenic chickens can also provide important tools for understanding and identifying agents which can affect, for example, embryogenesis and tissue formation. The term “chimeric animal” is used herein to refer to animals in which the recombinant gene is found, or in which the recombinant gene is expressed in some but not all cells of the animal. The term “tissue-specific chimeric animal” indicates that one of the recombinant MFGF genes is present and/or expressed or disrupted in some tissues but not others.

As used herein, the term “nucleic acid” refers to polynucleotides or oligonucleotides such as deoxyribonucleic acid (DNA), and, where appropriate, ribonucleic acid (RNA). The term should also be understood to include, as equivalents, analogs of either RNA or DNA made from nucleotide analogs and as applicable to the embodiment being described, single (sense or antisense) and double-stranded polynucleotides.

The term “polymorphism” refers to the coexistence of more than one form of a gene or portion (e.g., allelic variant) thereof A portion of a gene of which there are at least two different forms, i.e., two different nucleotide sequences, is referred to as a “polymorphic region of a gene”. A polymorphic region can be a single nucleotide, the identity of which differs in different alleles. A polymorphic region can also be several nucleotides long.

A “polymorphic gene” refers to a gene having at least one polymorphic region.

As used herein, the term “promoter” means a DNA sequence that regulates expression of a selected DNA sequence operably linked to the promoter, and which effects expression of the selected DNA sequence in cells. The term encompasses “tissue specific” promoters, i.e. promoters, which effect expression of the selected DNA sequence only in specific cells (e.g. cells of a specific tissue). The term also covers so-called “leaky” promoters, which regulate expression of a selected DNA primarily in one tissue, but cause expression in other tissues as well. The term also encompasses non-tissue specific promoters and promoters that constitutively express or that are inducible (i.e. expression levels can be controlled).

The terms “protein”, “polypeptide” and “peptide” are used interchangeably herein when referring to a gene product.

The term “recombinant protein” refers to a polypeptide of the present invention which is produced by recombinant DNA techniques, wherein generally, DNA encoding an MFGF polypeptide is inserted into a suitable expression vector which is in turn used to transform a host cell to produce the heterologous protein. Moreover, the phrase “derived from”, with respect to a recombinant MFGF gene, is meant to include within the meaning of “recombinant protein” those proteins having an amino acid sequence of a native MFGF polypeptide, or an amino acid sequence similar thereto which is generated by mutations including substitutions and deletions (including truncation) of a naturally occurring form of the polypeptide.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention to identity compounds that modulate an MFGF bioactivity.

As used herein, the term “specifically hybridizes” or “specifically detects” refers to the ability of a nucleic acid molecule of the invention to hybridize to at least approximately 6, 12, 20, 30, 50, 100, 150, 200, 300, 350, 400 or 425 consecutive nucleotides of a vertebrate, preferably an MFGF gene.

“Transcriptional regulatory sequence” is a generic term used throughout the specification to refer to DNA sequences, such as initiation signals, enhancers, and promoters, which induce or control transcription of protein coding sequences with which they are operably linked. In preferred embodiments, transcription of one of the MFGF genes is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring forms of MFGF polypeptide.

As used herein, the term “transfection” means the introduction of a nucleic acid, e.g., via an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of an MFGF polypeptide or, in the case of anti-sense expression from the transferred gene, the expression of a naturally-occurring form of the MFGF polypeptide is disrupted.

As used herein, the term “transgene” means a nucleic acid sequence (encoding, e.g., one of the MFGF polypeptides, or an antisense transcript thereto) which has been introduced into a cell. A transgene could be partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can also be present in a cell in the form of an episome. A transgene can include one or more transcriptional regulatory sequences and any other nucleic acid, such as introns, that may be necessary for optimal expression of a selected nucleic acid.

A “transgenic animal” refers to any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of the MFGF polypeptides, e.g. either agonistic or antagonistic forms. However, transgenic animals in which the recombinant MFGF gene is silent are also contemplated, as for example, the FLP or CRE recombinase dependent constructs described below. Moreover, “transgenic animal” also includes those recombinant animals in which gene disruption of one or more MFGF genes is caused by human intervention, including both recombination and antisense techniques.

The term “treating” as used herein is intended to encompass curing as well as ameliorating at least one symptom of the condition or disease.

The term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

4.3. Nucleic Acids of the Present Invention

The invention provides MFGF nucleic acids, homologs thereof, and portions thereof Preferred nucleic acids have a sequence at least about 60%, 61%, 62%, 63%, 64%, 65%, 66%, 67%, 68%, 69%, 70%, 71%, 72%, 73%, 74%, 75%, 76%, 77%, 78%, 79%, 80%, and more preferably 85% homologous and more preferably 90% and even more preferably at least 99% homologous with a nucleotide sequence of an MFGF gene, e.g., such as a sequence shown in one of SEQ ID NOS: 1, 3, 4, or 6 or complement thereof of the MFGF nucleic acids having ATCC Designation No. 209574 or No. 209648. Nucleic acids at least 90%, more preferably 95%, and most preferably at least about 98-99% homologous with a nucleic sequence represented in one of SEQ ID NOS. 1, 3, 4, or 6, or complement thereof are of course also within the scope of the invention. In preferred embodiments, the nucleic acid is mammalian and in particularly preferred embodiments, includes all or a portion of the nucleotide sequence corresponding to the coding region of one of SEQ ID NOS. 1, 3, 4, or 6.

The invention also pertains to isolated nucleic acids comprising a nucleotide sequence encoding MFGF polypeptides, variants and/or equivalents of such nucleic acids. The term equivalent is understood to include nucleotide sequences encoding functionally equivalent MFGF polypeptides or functionally equivalent peptides having an activity of an MFGF protein such as described herein. Equivalent nucleotide sequences will include sequences that differ by one or more nucleotide substitution, addition or deletion, such as allelic variants; and will, therefore, include sequences that differ from the nucleotide sequence of the MFGF gene shown in SEQ ID NOS. 1, 3, 4, or 6 due to the degeneracy of the genetic code.

Preferred nucleic acids are vertebrate MFGF nucleic acids. Particularly preferred vertebrate MFGF nucleic acids are mammalian. Regardless of species, particularly preferred MFGF nucleic acids encode polypeptides that are at least 60%, 65%, 70%, 72%, 74%, 76%, 78%, 80%, 90%, or 95% similar or identical to an amino acid sequence of a vertebrate MFGF protein. In one embodiment, the nucleic acid is a cDNA encoding a polypeptide having at least one bio-activity of the subject MFGF polypeptide. Preferably, the nucleic acid includes all or a portion of the nucleotide sequence corresponding to the nucleic acid of SEQ ID NOS. 1, 3, 4, or 6.

Still other preferred nucleic acids of the present invention encode an MFGF polypeptide which is comprised of at least 2, 5, 10, 25, 50, 100, 150 or 200 amino acid residues. For example, such nucleic acids can comprise about 50, 60, 70, 80, 90, or 100 base pairs. Also within the scope of the invention are nucleic acid molecules for use as probes/primer or antisense molecules (i.e. noncoding nucleic acid molecules), which can comprise at least about 6, 12, 20, 30, 50, 60, 70, 80, 90 or 100 base pairs in length.

Another aspect of the invention provides a nucleic acid which hybridizes under stringent conditions to a nucleic acid represented by SEQ ID NOS. 1, 3, 4, or 6 or complement thereof or the nucleic acids having ATCC Designation No. 209574 or No. 209648. Appropriate stringency conditions which promote DNA hybridization, for example, 6.0× sodium chloride/sodium citrate (SSC) at about 45° C., followed by a wash of 2.0× SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0× SSC at 50° C. to a high stringency of about 0.2× SSC at 50° C. In addition, the temperature in the wash step can be increased from low stringency conditions at room temperature, about 22° C., to high stringency conditions at about 65° C. Both temperature and salt may be varied, or temperature and salt concentration may be held constant while the other variable is changed. In a preferred embodiment, an MFGF nucleic acid of the present invention will bind to one of SEQ ID NOS. 1, 3, 4, or 6 or complement thereof under moderately stringent conditions, for example at about 2.0× SSC and about 40° C. In a particularly preferred embodiment, an MFGF nucleic acid of the present invention will bind to one of SEQ ID NOS. 1, 3, 4, or 6 or complement thereof under high stringency conditions.

Nucleic acids having a sequence that differs from the nucleotide sequences shown in one of SEQ ID NOS. 1, 3, 4, or 6 or complement thereof due to degeneracy in the genetic code are also within the scope of the invention. Such nucleic acids encode functionally equivalent peptides (i.e., peptides having a biological activity of an MFGF polypeptide) but differ in sequence from the sequence shown in the sequence listing due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence of an MFGF polypeptide. However, it is expected that DNA sequence polymorphisms that do lead to changes in the amino acid sequences of the subject MFGF polypeptides will exist among mammals. One skilled in the art will appreciate that these variations in one or more nucleotides (e.g., up to about 3-5% of the nucleotides) of the nucleic acids encoding polypeptides having an activity of an MFGF polypeptide may exist among individuals of a given species due to natural allelic variation.

Nucleic acids of the invention can encode one or more of the following domains of an MFGF protein: the signal peptide, the transmembrane domain, the extracellular domain, the heparin binding basic region, and the FGFR binding domain. The amino acid sequences of these domains in human MFGF (SEQ ID NO. 2) and the position of the nucleotide sequence in SEQ ID NO. 1 encoding these domains are indicated in Table Table I:

TABLE I

| | Position of Domains in | |
|---|---|---|
| | Human MFGF Nucleotides | Amino acids of |
| Domain | of SEQ ID NO. 1 | SEQ ID NO. 2 |
| signal sequence | 86 to 169 | 1 to 28 |
| extracellular domain | 170 to 706 | 29 to 207 |
| heparin binding basic | 545 to 577 | 154 to 164 |
| region | | |
| FGFR binding region | 182 to 199 | 33 to 38 |
| (i) | | |
| FGFR binding region | 539 to 568 | 152 to 161 |
| (ii) | | |

The polynucleotide sequence of the present invention may encode a mature form of the MFGF, i.e., a polypeptide substantially corresponding to about amino acids 29 to 207 of SEQ ID NO. 2 or SEQ ID NO. 5. This corresponds to a form of MFGF which does not comprise the leader peptide, e.g., an MFGF protein which does not comprise about amino acids 1 to 28 of SEQ ID NO. 2 or SEQ ID NO. 5.

The polynucleotide sequence of the present invention may encode a recombinant soluble form of MFGF, e.g. a polypeptide substantially corresponding to about amino acids 29 to 207 of SEQ ID NO. 2 or SEQ ID NO 5. This form of the protein may be obtained by deleting the nucleic acid sequences which encode the hydrophobic signal sequence which spans about amino acids 1 to 28 of SEQ ID NO. 2 or SEQ ID NO. 5, such that the resulting protein is a recombinant soluble form of MFGF without a hydrophobic signal sequence.

The polynucleotide of the present invention may also be fused in frame to a marker sequence, also referred to herein as “Tag sequence” encoding a “Tag peptide”, which allows for marking and/or purification of the polypeptide of the present invention. In a preferred embodiment, the marker sequence is a hexahistidine tag, e.g., supplied by a PQE-9 vector. Numerous other Tag peptides are available commercially. Other frequently used Tags include myc-epitopes (e.g., see Ellison et al. (1991) J Biol Chem 266:21150-21157) which includes a 10-residue sequence from c-myc, the pFLAG system (International Biotechnologies, Inc.), the pEZZ-protein A system (Pharmacia, N.J.), and a 16 amino acid portion of the Haemophilus influenza hemagglutinin protein. Furthermore, any polypeptide can be used as a Tag so long as a reagent, e.g., an antibody interacting specifically with the Tag polypeptide is available or can be prepared or identified.

In another embodiment, a fusion gene coding for a purification leader sequence, such as a poly-(His)/enterokinase cleavage site sequence at the N-terminus of the desired portion of the recombinant protein, can allow purification of the expressed fusion protein by affinity chromatography using a Ni²⁺ metal resin. The purification leader sequence can then be subsequently removed by treatment with enterokinase to provide the purified protein (e.g., see Hochuli et al. (1987) J. Chromatography 411:177; and Janknecht et al. PNAS 88:8972).

Techniques for making fusion genes are known to those skilled in the art. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al. John Wiley & Sons: 1992).

Other preferred MFGF fusion proteins include MFGF-immunoglobulin (MFGF-Ig) polypeptides. An MFGF-Ig polypeptide can comprise the entire extracellular domain of MFGF, e.g, human MFGF, or a variant thereof For example, an MFGF-Ig polypeptide can comprise an amino acid sequence from about amino acid 1 to about amino acid 207 of SEQ ID NOS. 2 or 5. MFGF-Ig fusion proteins can be prepared as described e.g., in U.S. Pat. No. 5,434,131.

As indicated by the examples set out below, MFGF protein-encoding nucleic acids can be obtained from mRNA present in any of a number of eukaryotic cells, e.g., from cardiac tissue. It should also be possible to obtain nucleic acids encoding MFGF polypeptides of the present invention from genomic DNA from both adults and embryos. For example, a gene encoding an MFGF protein can be cloned from either a cDNA or a genomic library in accordance with protocols described herein, as well as those generally known to persons skilled in the art. cDNA encoding an MFGF protein can be obtained by isolating total mRNA from a cell, e.g., a vertebrate cell, a mammalian cell, or a human cell, including embryonic cells. Double stranded cDNAs can then be prepared from the total mRNA, and subsequently inserted into a suitable plasmid or bacteriophage vector using any one of a number of known techniques. The gene encoding an MFGF protein can also be cloned using established polymerase chain reaction techniques in accordance with the nucleotide sequence information provided by the invention The nucleic acid of the invention can be DNA or RNA or analogs thereof A preferred nucleic acid is a cDNA represented by a sequence selected from the group consisting of SEQ ID NOS. 1, 3, 4, or 6.

Preferred nucleic acids encode a vertebrate MFGF polypeptide comprising an amino acid sequence that is at least about 60% homologous, more preferably at least about 70% homologous and most preferably at least about 80% homologous with an amino acid sequence contained in SEQ ID NOS. 2 or 5. Nucleic acids which encode polypeptides with at least about 90%, more preferably at least about 95%, and most preferably at least about 98-99% homology with an amino acid sequence represented in SEQ ID NO. 2 or 5 are also within the scope of the invention. In one embodiment, the nucleic acid is a cDNA encoding a peptide having at least one activity of the subject vertebrate MFGF polypeptide. Preferably, the nucleic acid includes all or a portion of the nucleotide sequence corresponding to the coding region of SEQ ID NOS. 1, 3, 4 or 6.

Preferred nucleic acids encode a bioactive fragment of a vertebrate MFGF polypeptide comprising an amino acid sequence, which is at least about 60% homologous or identical, more preferably at least about 70% homologous or identical, still more preferably at least about 75% homologous or identical and most preferably at least about 80% homologous or identical with an amino acid sequence of SEQ ID NOS. 2 or 5. Nucleic acids which encode polypeptides which are at least about 90%, more preferably at least about 95%, and most preferably at least about 98-99% homologous or identical, with an amino acid sequence represented in SEQ ID NOS. 2 or 5 are also within the scope of the invention.

Bioactive fragments of MFGF polypeptides can be polypeptides having one or more of the following biological activities: heparin sulfate binding activity, heparin sulfate proteoglycan binding activity, FGFR binding activity, mitogenic activity, chemotactic activity, cellular transformation activity, cellular differentiation inducing activity, angiogenic activity, neurogenic activity, or mesoderm inducing activity. Furthermore these fragments can either promote or inhibit these processes or agonize or antagonize the activity of another agent which itself promotes or inhibits these processes. Assays for determining whether an MFGF polypeptide has any of these or other biological activities are known in the art and are further described herein.

For example, nucleic acids encoding proteins having an MFGF activity include nucleic acids comprising a nucleotide sequence encoding a heparin sulfate binding region, such as the region of MFGF consisting of about amino acids 149 to 169 of SEQ ID NOS. 2 or 5. Such a nucleic acid can be represented by the generic formula: X-D-Y, wherein D represents nucleotides 530 to 592 of SEQ ID NO. 1 or nucleotides 446 to 508 of SEQ ID NO. 4, and X and Y represent a certain number of nucleotides located 5′ and 3′ of the sequence represented by D, respectively. For example, a nucleic acid of the invention can comprise nucleotides 530 to 592 of SEQ ID NO. 1 or nucleotides 446 to 508 of SEQ ID NO. 4 and X and Y selected from any of 0, 5, 10, 20, 30, 50, 100, 150, 200, 300, 400, 500, 600, 700, 800, 900, or about 1000 nucleotides.

Additional nucleic acids included in the present invention are those encoding a bioactive fragment of MFGF include nucleic acids comprising a nucleotide sequence encoding a fibroblast growth factor receptor (FGFR) binding site, such as the site of MFGF consisting of amino acids 33 to 45 of SEQ ID NOS. 2 or 5. Such a nucleic acid can be represented by the generic formula: X-D-Y, wherein D represents nucleotides 182 to 220 of SEQ ID NO. 1 or nucleotides 98 to 136 of SEQ ID NO. 4, and X and Y represent a certain number of nucleotides located 5′ and 3′ of the sequence represented by D respectively. For example, a nucleic acid of the invention can comprise nucleotides 182 to 220 of SEQ ID NO. 1 or nucleotides 98 to 136 of SEQ ID NO. 4 and X and Y selected from any of 0, 5, 10, 20, 30, 50, 100, 150, 200, 300, 400, 500, 600, 700, 800, 900, or about 1000 nucleotides.

Nucleic acids encoding modified forms or mutant forms of MFGF also include those encoding MFGF proteins having mutated glycosylation sites, such that either the encoded MFGF protein is not glycosylated, partially glycosylated and/or has a modified glycosylation pattern. Two potential N-linked glycosylation sites have been identified in MFGF and these are located within amino acids 39 to 42 and 137 to 140 as shown in SEQ ID NO. 2 for hMFGF and in SEQ ID NO. 5 for MFGF. Glycosylation sites, N-glycosylation or O-glycosylation sites can also be added to the protein. Amino acid sequence motifs required for the attachment of a sugar unit are well known in the art.

Other preferred nucleic acids of the invention include nucleic acids encoding derivatives of MFGF polypeptides which lack one or more biological activities of MFGF polypeptides. Such nucleic acids can be obtained, e.g., by a first round of screening of libraries for the presence or absence of a first activity and a second round of screening for the presence or absence of another activity. For example, it has been shown that interaction of FGF-2 (bFGF), FGF-1 (aFGF), and FGF4 (K-FGF) with heparin sulfate is necessary for their interaction with FGFRs, perhaps because of a conformational change induced by FGF binding to heparin (Yayon, et al. (1991) Cell 64: 841-848). Therefore the products of a screen to identify suitable nucleic acid molecules encoding MFGF polypeptide fragments which bind to heparin sulfate on the one hand, could subsequently be reexamined in a second round of screening for the ability to inhibit MFGF-dependent activation of FGFRs by virtue of their ability to compete with biologically active heparin sulfate-bound wild-type MFGF for binding to FGFRs.

Also within the scope of the invention are nucleic acids encoding splice variants or nucleic acids representing transcripts synthesized from an alternative transcriptional initiation site, such as those whose transcription was initiated from a site in an intron. For example, cloning and analysis of murine and human FGF-8 genes has revealed the existence of multiple potential splice variants of the encoded transcripts (Gemel, J. et al. (1996) Genomics 35: 253-257). Such homologs can be cloned by hybridization or PCR, as further described herein.

In preferred embodiments, the MFGF nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup B. et al. (1996)Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. PNAS 93: 14670-675.

PNAs of MFGF can be used in therapeutic and diagnostic applications and are further described herein in section 4.3.2. Such modified nucleic acids can be used as antisense or antigene agents for sequence-specific modulation of gene expression or in the analysis of single base pair mutations in a gene by, e.g., PNA directed PCR clamping or as probes or primers for DNA sequence and hybridization (Hyrup B. et al (1996) supra; Perry-O'Keefe supra).

PNAs of MFGF can further be modified, e.g., to enhance their stability or cellular uptake, e.g., by attaching lipophilic or other helper groups to the MFGF PNA, by the formation of PNA-DNA chimeras, or by the use of liposomes or other techniques of drug delivery known in the art. MFGF PNAs can also be linked to DNA as described, e.g., in Hyrup B. (1996) supra and Finn P. J. etal. (1996) Nucleic Acids Research 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment Finn P. J. et al. (1996) supra). Alternatively, chimeric molecules can be synthesized with a 5′ DNA segment and a 3′ PNA segment (Peterser, K. H. et al. (1975) Bioorganic Med Chem. Lett. 5: 1119-11124).

In other embodiments, MFGF nucleic acids may include other appended groups such as peptides (e.g., for targeting host cell receptors in vivo), or agents that facilitate transport across the cell membrane as described in section 4.3.2. herein.

4.3.1 Probes and Primers

The nucleotide sequences determined from the cloning of MFGF genes from mammalian organisms will further allow for the generation of probes and primers designed for use in identifying and/or cloning MFGF homologs in other cell types, e.g., from other tissues, as well as MFGF homologs from other mammalian organisms. For instance, the present invention also provides a probe/primer comprising a substantially purified oligonucleotide, which oligonucleotide comprises a region of nucleotide sequence that hybridizes under stringent conditions to at least approximately 12, preferably 25, more preferably 40, 50 or 75 consecutive nucleotides of sense or anti-sense sequence selected from the group consisting of SEQ ID NOS. 1, 3, 4, or 6 or naturally occurring mutants thereof. For instance, primers based on the nucleic acid represented in SEQ ID NOS. 1 or 3 can be used in PCR reactions to clone MFGF homologs.

Likewise, probes based on the subject MFGF sequences can be used to detect transcripts or genomic sequences encoding the same or homologous proteins, for use, e.g, in prognostic or diagnostic assays (further described below). In preferred embodiments, the probe further comprises a label group attached thereto and able to be detected, e.g., the label group is selected from amongst radioisotopes, fluorescent compounds, enzymes, and enzyme co-factors.

Probes and primers can be prepared and modified, e.g., as previously described herein for other types of nucleic acids.

4.3.2 Antisense, Ribozyme and Triplex Techniques

Another aspect of the invention relates to the use of the isolated nucleic acid in “antisense” therapy. As used herein, “antisense” therapy refers to administration or in situ generation of oligonucleotide molecules or their derivatives which specifically hybridize (e.g., bind) under cellular conditions, with the cellular mRNA and/or genomic DNA encoding one or more of the subject MFGF proteins so as to inhibit expression of that protein, e.g., by inhibiting transcription and/or translation. The binding may be by conventional base pair complementarity, or, for example, in the case of binding to DNA duplexes, through specific interactions in the major groove of the double helix. In general, “antisense” therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the cellular mRNA which encodes an MFGF protein. Alternatively, the antisense construct is an oligonucleotide probe which is generated ex vivo and which, when introduced into the cell causes inhibition of expression by hybridizing with the mRNA and/or genomic sequences of an MFGF gene. Such oligonucleotide probes are preferably modified oligonucleotides which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668. With respect to antisense DNA, oligodeoxyribonucleotides derived from the translation initiation site, e.g., between the −10 and +10 regions of the MFGF nucleotide sequence of interest, are preferred.

Antisense approaches involve the design of oligonucleotides (either DNA or RNA) that are complementary to MFGF mRNA. The antisense oligonucleotides will bind to the MFGF mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. (Wagner, R 1994. Nature 372:333). Therefore, oligonucleotides complementary to either the 5′ or 3′ untranslated, non-coding regions of an MFGF gene could be used in an antisense approach to inhibit translation of endogenous MFGF mRNA. Oligonucleotides complementary to the 5′ untranslated region of the mRNA should include the complement of the AUG start codon. Antisense oligonucleotides complementary to mRNA coding regions are less efficient inhibitors of translation but could also be used in accordance with the invention. Whether designed to hybridize to the 5′, 3′ or coding region of MFGF mRNA, antisense nucleic acids should be at least six nucleotides in length, and are preferably less than about 100 and more preferably less than about 50, 25, 17 or 10 nucleotides in length.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A 86:6553-6556; Lemaitre et al., 1987, Proc. Natl. Acad. Sci. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluraci 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their ability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate olgonucleotides can be prepared by use of controlled pore glass polymer supports (Sarin et al., 1988, Proc. Natl. Acad. Sci. U.S.A. 85:7448-7451), etc.

While antisense nucleotides complementary to the MFGF coding region sequence can be used, those complementary to the transcribed untranslated region and to the region comprising the initiating methionine are most preferred.

The antisense molecules can be delivered to cells which express MFGF in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

However, it may be difficult to achieve intracellular concentrations of the antisense sufficient to suppress translation on endogenous mRNAs in certain instances. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous MFGF transcripts and thereby prevent translation of the MFGF mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive and can include but not be limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:3942), etc. Any type of plasmid, cosmid, YAC or viral vector can be used to prepare the recombinant DNA construct which can be introduced directly into the tissue site. Alternatively, viral vectors can be used which selectively infect the desired tissue, in which case administration may be accomplished by another route (e.g., systematically).

Ribozyme molecules designed to catalytically cleave MFGF mRNA transcripts can also be used to prevent translation of MFGF mRNA and expression of MFGF (See, e.g., PCT International Publication WO90/11364, published Oct. 4, 1990; Sarver et al., 1990, Science 247:1222-1225 and U.S. Pat. No. 5,093,246). While ribozymes that cleave mRNA at site specific recognition sequences can be used to destroy MFGF mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591. There are a number of potential hammerhead ribozyme cleavage sites within the nucleotide sequence of human MFGF cDNA (FIG. 1) and the murine MFGF cDNA (FIG. 2). Preferably the ribozyme is engineered so that the cleavage recognition site is located near the 5′ end of the MFGF mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470475; Zaug, et al., 1986, Nature, 324:429-433; published International patent application No. WO88/04300 by University Patents Inc.; Been and Cech, 1986, Cell, 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in an MFGF gene.

As in the antisense approach, the ribozymes can be composed of modified oligonucleotides (e.g., for improved stability, targeting, etc.) and should be delivered to cells which express the MFGF gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous MFGF messages and inhibit translation. Because ribozymes unlike antisense molecules, are catalytic, a lower intracellular concentration is required for efficiency.

Endogenous MFGF gene expression can also be reduced by inactivating or “knocking out” the MFGF gene or its promoter using targeted homologous recombination. (E.g., see Smithies et al., 1985, Nature 317:230-234; Thomas & Capecchi, 1987, Cell 51:503-512; Thompson et al., 1989 Cell 5:313-321; each of which is incorporated by reference herein in its entirety). For example, a mutant, non-functional MFGF (or a completely unrelated DNA sequence) flanked by DNA homologous to the endogenous MFGF gene (either the coding regions or regulatory regions of the MFGF gene) can be used, with or without a selectable marker and/or a negative selectable marker, to transfect cells that express MFGF in vivo. Insertion of the DNA construct, via targeted homologous recombination, results in inactivation of the MFGF gene. Such approaches are particularly suited in the agricultural field where modifications to ES (embryonic stem) cells can be used to generate animal offspring with an inactive MFGF (e.g., see Thomas & Capecchi 1987 and Thompson 1989, supra). However this approach can be adapted for use in humans provided the recombinant DNA constructs are directly administered or targeted to the required site in vivo using appropriate viral vectors.

Alternatively, endogenous MFGF gene expression can be reduced by targeting deoxyribonucleotide sequences complementary to the regulatory region of the MFGF gene (i.e., the MFGF promoter and/or enhancers) to form triple helical structures that prevent transcription of the MFGF gene in target cells in the body. (See generally, Helene, C. 1991, Anticancer Drug Des., 6(6):569-84; Helene, C., et al., 1992, Ann. N.Y. Acad. Sci., 660:27-36; and Maher, L. J., 1992, Bioassays 14(12):807-15).

Nucleic acid molecules to be used in triple helix formation for the inhibition of transcription are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

Antisense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

4.3.3. Vectors Encoding MFGF Proteins and MFGF Expressing Cells

The invention further provides plasmids and vectors encoding an MFGF protein, which can be used to express an MFGF protein in a host cell. The host cell may be any prokaryotic or eukaryotic cell. Thus, a nucleotide sequence derived from the cloning of mammalian MFGF proteins, encoding all or a selected portion of the full-length protein, can be used to produce a recombinant form of an MFGF polypeptide via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial) cells, are standard procedures well known in the art.

Vectors that allow expression of a nucleic acid in a cell are referred to as expression vectors. Typically, expression vectors used for expressing an MFGF protein contain a nucleic acid encoding an MFGF polypeptide, operably linked to at least one transcriptional regulatory sequence. Regulatory sequences are art-recognized and are selected to direct expression of the subject MFGF proteins. Transcriptional regulatory sequences are described in Goeddel; Gene Expression Technology: Methods in Enzymology 185, Academic Press, San Diego, Calif. (1990). In one embodiment, the expression vector includes a recombinant gene encoding a peptide having an agonistic activity of a subject MFGF polypeptide, or alternatively, encoding a peptide which is an antagonistic form of an MFGF protein.

Suitable vectors for the expression of an MFGF polypeptide include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIPS, YEP51, YEP52, pYES2, and YRP17 are cloning and expression vehicles useful in the introduction of genetic constructs into S. cerevisiae (see, for example, Broach et al. (1983) in Experimental Manipulation of Gene Expression, ed. M. Inouye Academic Press, p. 83, incorporated by reference herein). These vectors can replicate in E. coli due the presence of the pBR322 ori, and in S. cerevisiae due to the replication determinant of the yeast 2 micron plasmid. In addition, drug resistance markers such as ampicillin can be used. In an illustrative embodiment, an MFGF polypeptide is produced recombinantly utilizing an expression vector generated by sub-cloning the coding sequence of one of the MFGF genes represented in SEQ ID NOS. 1 or 3.

The preferred mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papillomavirus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2^{nd} Ed., ed. by Sambrook, Fritsch and Maniatis (Cold Spring Harbor Laboratory Press: 1989) Chapters 16 and 17.

In some instances, it may be desirable to express the recombinant MFGF polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the B-gal containing pBlueBac III)

When it is desirable to express only a portion of an MFGF protein, such as a form lacking a portion of the N-terminus, i.e. a truncation mutant which lacks the signal peptide, it may be necessary to add a start codon (ATG) to the oligonucleotide fragment containing the desired sequence to be expressed. It is well known in the art that a methionine at the N-terminal position can be enzymatically cleaved by the use of the enzyme methionine aminopeptidase (MAP). MAP has been cloned from E. coli (Ben-Bassat et al. (1987) J. Bacteriol. 169:751-757) and Salmonella typhimurium and its in vitro activity has been demonstrated on recombinant proteins (Miller et al. (1987) PNAS 84:2718-1722). Therefore, removal of an N-terminal methionine, if desired, can be achieved either in vivo by expressing MFGF derived polypeptides in a host which produces MAP (e.g., E. coli or CM89 or S. cerevisiae), or in vitro by use of purified MAP (e.g., procedure of Miller et al., supra).

Moreover, the gene constructs of the present invention can also be used as part of a gene therapy protocol to deliver nucleic acids encoding either an agonistic or antagonistic form of one of the subject MFGF proteins. Thus, another aspect of the invention features expression vectors for in vivo or in vitro transfection and expression of an MFGF polypeptide in particular cell types so as to reconstitute the function of, or alternatively, abrogate the function of MFGF in a tissue. This could be desirable, for example, when the naturally-occurring form of the protein is misexpressed or the natural protein is mutated and less active.

In addition to viral transfer methods, non-viral methods can also be employed to cause expression of a subject MFGF polypeptide in the tissue of an animal. Most nonviral methods of gene transfer rely on normal mechanisms used by mammalian cells for the uptake and intracellular transport of macromolecules. In preferred embodiments, non-viral targeting means of the present invention rely on endocytic pathways for the uptake of the subject MFGF polypeptide gene by the targeted cell. Exemplary targeting means of this type include liposomal derived systems, poly-lysine conjugates, and artificial viral envelopes.

In other embodiments transgenic animals, described in more detail below could be used to produce recombinant proteins.

4.4. Polypeptides of the Present Invention

The present invention makes available isolated MFGF polypeptides which are isolated from, or otherwise substantially free of other cellular proteins. The term “substantially free of other cellular proteins” (also referred to herein as “contaminating proteins”) or “substantially pure or purified preparations” are defined as encompassing preparations of MFGF polypeptides having less than about 20% (by dry weight) contaminating protein, and preferably having less than about 5% contaminating protein. Functional forms of the subject polypeptides can be prepared, for the first time, as purified preparations by using a cloned gene as described herein.

Preferred MFGF proteins of the invention have an amino acid sequence which is at least about 60%, 65%, 66%, 67%, 68%, 69%, 70%, 71%, 72%, 73%, 74%, 75%, 76%,77%, 78%,790%, 80%, 85%, 90%, or 95% identical or homologous to an amino acid sequence of SEQ ID NO. 2. Even more preferred MFGF proteins comprise an amino acid sequence which is at least about 97, 98, or 99% homologous or identical to an amino acid sequence of SEQ ID NOS. 2 or 5. Such proteins can be recombinant proteins, and can be, e.g., produced in vitro from nucleic acids comprising a nucleotide sequence set forth in SEQ ID NOS. 1, 3, 4, or 6, or homologs thereof. For example, recombinant polypeptides preferred by the present invention can be encoded by a nucleic acid, which is at least 85% homologous and more preferably 90% homologous and most preferably 95% homologous with a nucleotide sequence set forth in SEQ ID NOS. 1, 3, 4, or 6. Polypeptides which are encoded by a nucleic acid that is at least about 98-99% homologous with the sequence of SEQ ID NOS. 1, 3, 4, or 6 are also within the scope of the invention.

In a preferred embodiment, an MFGF protein of the present invention is a mammalian MFGF protein. In a particularly preferred embodiment an MFGF protein is set forth as SEQ ID NO. 2 or SEQ ID NO. 5. In particularly preferred embodiments, an MFGF protein has an MFGF bioactivity. It will be understood that certain post-translational modifications, e.g., phosphorylation and the like, can increase the apparent molecular weight of the MFGF protein relative to the unmodified polypeptide chain.

The invention also features protein isoforms encoded by splice variants of the present invention. Such isoforms may have biological activities identical to or different from those possessed by the MFGF proteins specified by SEQ ID NOS. 2 or 5. For example, analysis of three different isoforms of FGF-8 has revealed significant differences in the potency of NIH3T3 cell transformation and tumorigenicity of the transfected cells in nude mice (MacArthur, C. A et al. (1995) Cell Growth and Differentiation 6: 817-35).

MFGF polypeptides preferably are capable of functioning as either an agonist or antagonist of at least one biological activity of a wild-type (“authentic”) MFGF protein of the appended sequence listing. The term “evolutionarily related to”, with respect to amino acid sequences of MFGF proteins, refers to both polypeptides having amino acid sequences which have arisen naturally, and also to mutational variants of human MFGF polypeptides which are derived, for example, by combinatorial mutagenesis.

Full length proteins or fragments corresponding to one or more particular motifs and/or domains or to arbitrary sizes, for example, at least 5, 10, 25, 50, 75 and 100, amino acids in length are within the scope of the present invention.

For example, isolated MFGF polypeptides can be encoded by all or a portion of a nucleic acid sequence shown in any of SEQ ID NOS. 1, 3, 4, or 6. Isolated peptidyl portions of MFGF proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, an MFGF polypeptide of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of a wild-type (e.g., “authentic”) MFGF protein.

An MFGF polypeptide can be a membrane bound form or a soluble form. A preferred soluble MFGF polypeptide is a polypeptide which does not contain the hydrophobic signal sequence domain located from about amino acid 1 to about amino acid 228 of SEQ ID NOS. 2 or 5. This preferred embodiment encompasses a polypeptide substantially corresponding to about amino acids 29 to 207 of SEQ ID NOS. 2 or 5. It is likely that there are natural forms of MFGF which fail to contain this domain. Alternatively, such proteins can be created by genetic engineering by methods known in the art. Soluble MFGF proteins can comprise an amino acid sequence from about amino acid 29 to about amino acid 207 of SEQ ID NOS. 2 or 5 or homologs thereof Alternatively, soluble MFGF proteins can comprise the signal sequence, i.e., amino acids 1-28 of SEQ ID NO. 2 or 5, or a heterologous signal sequence, which is operably linked to the amino acid sequence of the mature processed form of MFGF corresponding to about amino acids 29 to 207 of SEQ ID NOS 2 or 5.

In general, polypeptides referred to herein as having an activity (e.g., are “bioactive”) of an MFGF protein are defined as polypeptides which include an amino acid sequence encoded by all or a portion of the nucleic acid sequences shown in one of SEQ ID NOS. 1, 3, 4, or 6 and which mimic or antagonize all or a portion of the biological/biochemical activities of a naturally occurring MFGF protein. Examples of such biological activity include: heparin sulfate binding activity, heparin sulfate proteoglycan binding activity, FGFR binding activity, mitogenic activity, chemotactic activity, cellular transformation activity, cellular differentiation inducing activity, angiogenic activity, neurogenic activity, or mesoderm inducing activity. Furthermore these fragments can either promote or inhibit these processes or agonize or antagonize the activity of another agent which itself promotes or inhibits these processes. Other biological activities of the subject MFGF proteins will be reasonably apparent to those skilled in the art. According to the present invention, a polypeptide has biological activity if it is a specific agonist or antagonist of a naturally-occurring form of an MFGF protein.

A preferred MFGF polypeptide having a biological activity is an MFGF polypeptide comprising a heparin sulfate binding domain, e.g, an amino acid sequence from amino acid 154 to amino acid 164 of SEQ ID NOS. 2 or 5.

Assays for determining whether a compound, e.g, a protein, such as an MFGF protein or variant thereof has one or more of the above biological activities are well known in the art.

Other preferred proteins of the invention are those encoded by the nucleic acids set forth in the section pertaining to nucleic acids of the invention. In particular, the invention provides fusion proteins, e.g., MFGF-immunoglobulin fusion proteins. Such fusion proteins can provide, e.g., enhanced stability and solubility of MFGF proteins and may thus be useful in therapy. Fusion proteins can also be used to produce an immunogenic fragment of an MFGF protein. For example, the VP6 capsid protein of rotavirus can be used as an immunologic carrier protein for portions of the MFGF polypeptide, either in the monomeric form or in the form of a viral particle. The nucleic acid sequences corresponding to the portion of a subject MFGF protein to which antibodies are to be raised can be incorporated into a fusion gene construct which includes coding sequences for a late vaccinia virus structural protein to produce a set of recombinant viruses expressing fusion proteins comprising MFGF epitopes as part of the virion. It has been demonstrated with the use of immunogenic fusion proteins utilizing the Hepatitis B surface antigen fusion proteins that recombinant Hepatitis B virions can be utilized in this role as well. Similarly, chimeric constructs coding for fusion proteins containing a portion of an MFGF protein and the poliovirus capsid protein can be created to enhance immunogenicity of the set of polypeptide antigens (see, for example, EP Publication No: 0259149; and Evans et al. (1989) Nature 339:385; Huang et al. (1988) J. Virol. 62:3855; and Schlienger et al. (1992) J. Virol. 66:2).

The Multiple antigen peptide system for peptide-based immunization can also be utilized to generate an immunogen, wherein a desired portion of an MFGF polypeptide is obtained directly from organo-chemical synthesis of the peptide onto an oligomeric branching lysine core (see, for example, Posnett et al. (1988) JBC 263:1719 and Nardelli et al. (1992) J. Immunol. 148:914). Antigenic determinants of MFGF proteins can also be expressed and presented by bacterial cells.

In addition to utilizing fusion proteins to enhance immunogenicity, it is widely appreciated that fusion proteins can also facilitate the expression of proteins, and accordingly, can be used in the expression of the MFGF polypeptides of the present invention. For example, MFGF polypeptides can be generated as glutathione-S-transferase (GST-fusion) proteins. Such GST-fusion proteins can enable easy purification of the MFGF polypeptide, as for example by the use of glutathione-derivatized matrices (see, for example, Current Protocols in Molecular Biology, eds. Ausubel et al. (N. Y.: John Wiley & Sons, 1991)).

The present invention further pertains to methods of producing the subject MFGF polypeptides. For example, a host cell transfected with a nucleic acid vector directing expression of a nucleotide sequence encoding the subject polypeptides can be cultured under appropriate conditions to allow expression of the peptide to occur. Suitable media for cell culture are well known in the art. The recombinant MFGF polypeptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide. In a preferred embodiment, the recombinant MFGF polypeptide is a fusion protein containing a domain which facilitates its purification, such as GST fusion protein.

Moreover, it will be generally appreciated that, under certain circumstances, it may be advantageous to provide homologs of one of the subject MFGF polypeptides which function in a limited capacity as one of either an MFGF agonist (mimetic) or an MFGF antagonist, in order to promote or inhibit only a subset of the biological activities of the naturally-occurring form of the protein. Thus, specific biological effects can be elicited by treatment with a homolog of limited function, and with fewer side effects relative to treatment with agonists or antagonists which are directed to all of the biological activities of naturally occurring forms of MFGF proteins.

Homologs of each of the subject MFGF proteins can be generated by mutagenesis, such as by discrete point mutation(s), or by truncation. For instance, mutation can give rise to homologs which retain substantially the same, or merely a subset, of the biological activity of the MFGF polypeptide from which it was derived. Alternatively, antagonistic forms of the protein can be generated which are able to inhibit the function of the naturally occurring form of the protein, such as by competitively binding to an MFGF receptor.

The recombinant MFGF polypeptides of the present invention also include homologs of the wildtype MFGF proteins, such as versions of those protein which are resistant to proteolytic cleavage, as for example, due to mutations which alter ubiquitination or other enzymatic targeting associated with the protein.

MFGF polypeptides may also be chemically modified to create MFGF derivatives by forming covalent or aggregate conjugates with other chemical moieties, such as glycosyl groups, lipids, phosphate, acetyl groups and the like. Covalent derivatives of MFGF proteins can be prepared by linking the chemical moieties to functional groups on amino acid sidechains of the protein or at the N-terminus or at the C-terminus of the polypeptide.

Modification of the structure of the subject MFGF polypeptides can be for such purposes as enhancing therapeutic or prophylactic efficacy, stability (e.g., ex vivo shelf life and resistance to proteolytic degradation), or post-translational modifications (e.g., to alter phosphorylation pattern of protein). Such modified peptides, when designed to retain at least one activity of the naturally-occurring form of the protein, or to produce specific antagonists thereof are considered functional equivalents of the MFGF polypeptides described in more detail herein. Such modified peptides can be produced, for instance, by amino acid substitution, deletion, or addition. The substitutional variant may be a substituted conserved amino acid or a substituted non-conserved amino acid.

For example, it is reasonable to expect that an isolated replacement of a leucine with an isoleucine or valine, an aspartate with a glutamate, a threonine with a serine, or a similar replacement of an amino acid with a structurally related amino acid (i.e. isosteric and/or isoelectric mutations) will not have a major effect on the biological activity of the resulting molecule. Conservative replacements are those that take place within a family of amino acids that are related in their side chains. Genetically encoded amino acids can be divided into four families: (1) acidic=aspartate, glutamate; (2) basis=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 2^{nd} ed., Ed. by L. Stryer, W H Freeman and Co.: 1981). Whether a change in the amino acid sequence of a peptide results in a functional MFGF homolog (e.g., functional in the sense that the resulting polypeptide mimics or antagonizes the wild-type form) can be readily determined by assessing the ability of the variant peptide to produce a response in cells in a fashion similar to the wild-type protein, or competitively inhibit such a response. Polypeptides in which more than one replacement has taken place can readily be tested in the same manner.

This invention further contemplates a method for generating sets of combinatorial mutants of the subject MFGF proteins as well as truncation mutants, and is especially useful for identifying potential variant sequences (e.g., homologs). The purpose of screening such combinatorial libraries is to generate, for example, novel MFGF homologs which can act as either agonists or antagonist, or alternatively, possess novel activities all together. Thus, combinatorially-derived homologs can be generated to have an increased potency relative to a naturally occurring form of the protein.

In one embodiment, the variegated library of MFGF variants is generated by combinatorial mutagenesis at the nucleic acid level, and is encoded by a variegated gene library. For instance, a mixture of synthetic oligonucleotides can be enzymatically ligated into gene sequences such that the degenerate set of potential MFGF sequences are expressible as individual polypeptides, or alternatively, as a set of larger fusion proteins (e.g., for phage display) containing the set of MFGF sequences therein.

There are many ways by which such libraries of potential MFGF homologs can be generated from a degenerate oligonucleotide sequence. Chemical synthesis of a degenerate gene sequence can be carried out in an automatic DNA synthesizer, and the synthetic genes then ligated into an appropriate expression vector. The purpose of a degenerate set of genes is to provide, in one mixture, all of the sequences encoding the desired set of potential MFGF sequences. The synthesis of degenerate oligonucleotides is well known in the art (see for example, Narang, S A (1983) Tetrahedron 39:3; Itakura et al. (1981) Recombinant DNA, Proc 3^{rd} Cleveland Sympos. Macromolecules, ed. A G Walton, Amsterdam: Elsevier pp 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

Likewise, a library of coding sequence fragments can be provided for an MFGF clone in order to generate a variegated population of MFGF fragments for screening and subsequent selection of bioactive fragments. A variety of techniques are known in the art for generating such libraries, including chemical synthesis. In one embodiment, a library of coding sequence fragments can be generated by (i) treating a double stranded PCR fragment of an MFGF coding sequence with a nuclease under conditions wherein nicking occurs only about once per molecule; (ii) denaturing the double stranded DNA; (iii) renaturing the DNA to form double stranded DNA which can include sense/antisense pairs from different nicked products; (iv) removing single stranded portions from reformed duplexes by treatment with S1 nuclease; and (v) ligating the resulting fragment library into an expression vector. By this exemplary method, an expression library can be derived which codes for N-terminal, C-terminal and internal fragments of various sizes.

A wide range of techniques are known in the art for screening gene products of combinatorial libraries made by point mutations or truncation, and for screening cDNA libraries for gene products having a certain property. Such techniques will be generally adaptable for rapid screening of the gene libraries generated by the combinatorial mutagenesis of MFGF homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate MFGF sequences created by combinatorial mutagenesis techniques. Combinatorial mutagenesis has a potential to generate very large libraries of mutant proteins, e.g., in the order of 10²⁶ molecules. Combinatorial libraries of this size may be technically challenging to screen even with high throughput screening assays. To overcome this problem, a new technique has been developed recently, recrusive ensemble mutagenesis (REM), which allows one to avoid the very high proportion of non-functional proteins in a random library and simply enhances the frequency of functional proteins, thus decreasing the complexity required to achieve a useful sampling of sequence space. REM is an algorithm which enhances the frequency of functional mutants in a library when an appropriate selection or screening method is employed (Arkin and Yourvan, 1992, PNAS USA 89:7811-7815; Yourvan et al., 1992, Parallel Problem Solving from Nature, 2., In Maenner and Manderick, eds., Elsevir Publishing Co., Amsterdam, pp. 401-410; Delgrave et al., 1993, Protein Engineering 6(3):327-331).

The invention also provides for reduction of the MFGF proteins to generate mimetics, e.g., peptide or non-peptide agents, such as small molecules, which are able to disrupt binding of an MFGF polypeptide of the present invention with a molecule, e.g. target peptide. Thus, such mutagenic techniques as described above are also useful to map the determinants of the MFGF proteins which participate in protein-protein interactions involved in, for example, binding of the subject MFGF polypeptide to a target peptide. To illustrate, the critical residues of a subject MFGF polypeptide which are involved in molecular recognition of its receptor can be determined and used to generate MFGF derived peptidomimetics or small molecules which competitively inhibit binding of the authentic MFGF protein with that moiety. By employing, for example, scanning mutagenesis to map the amino acid residues of the subject MFGF proteins which are involved in binding other proteins, peptidomimetic compounds can be generated which mimic those residues of the MFGF protein which facilitate the interaction. Such mimetics may then be used to interfere with the normal function of an MFGF protein. For instance, non-hydrolyzable peptide analogs of such residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9^{th} American Peptide Symposium) Pierce Chemical Co. Rockland, Ill., 1985), b-turn dipeptide cores (Nagai et al. (1985) Tetrahedron Lett 26:647; and Sato et al. (1986) J Chem Soc Perkin Trans 1:1231), and b-aminoalcohols (Gordon et al. (1985) Biochem Biophys Res Commun 126:419; and Dann et al. (1986) Biochem Biophys Res Commun 134:71).

4.5. Anti-MFGF Antibodies and Uses Therefor

Another aspect of the invention pertains to an antibody specifically reactive with a mammalian MFGF protein, e.g., a wild-type or mutated MFGF protein. For example, by using immunogens derived from an MFGF protein, e.g., based on the cDNA sequences, anti-protein/anti-peptide antisera or monoclonal antibodies can be made by standard protocols (See, for example, Antibodies: A Laboratory Manual ed. by Harlow and Lane (Cold Spring Harbor Press: 1988)). A mammal, such as a mouse, a hamster or rabbit can be immunized with an immunogenic form of the peptide (e.g., a mammalian MFGF polypeptide or an antigenic fragment which is capable of eliciting an antibody response, or a fusion protein as described above). Techniques for conferring immunogenicity on a protein or peptide include conjugation to carriers or other techniques well known in the art. An immunogenic portion of an MFGF protein can be administered in the presence of adjuvant. The progress of immunization can be monitored by detection of antibody titers in plasma or serum. Standard ELISA or other immunoassays can be used with the immunogen as antigen to assess the levels of antibodies. In a preferred embodiment, the subject antibodies are immunospecific for antigenic determinants of an MFGF protein of a mammal, e.g., antigenic determinants of a protein set forth in SEQ ID No: 2 or closely related homologs (e.g., at least 90% homologous, and more preferably at least 94% homologous).

Following immunization of an animal with an antigenic preparation of an MFGF polypeptide, anti-MFGF antisera can be obtained and, if desired, polyclonal anti-MFGF antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique originally developed by Kohler and Milstein ((1975) Nature, 256: 495497), the human B cell hybridoma technique (Kozbar et al., (1983) Immunology Today , 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a mammalian MFGF polypeptide of the present invention and monoclonal antibodies isolated from a culture comprising such hybridoma cells. In one embodiment anti-human MFGF antibodies specifically react with the protein encoded by a nucleic acid having SEQ ID NO. 1 or 4.

The term antibody as used herein is intended to include fragments thereof which are also specifically reactive with one of the subject mammalian MFGF polypeptides. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific, single-chain, and chimeric and humanized molecules having affinity for an MFGF protein conferred by at least one CDR region of the antibody. In preferred embodiments, the antibody further comprises a label attached thereto and able to be detected, (e.g., the label can be a radioisotope, fluorescent compound, enzyme or enzyme co-factor).

Anti-MFGF antibodies can be used, e.g., to monitor MFGF protein levels in an individual for determining, e.g., whether a subject has a disease or condition associated with an aberrant MFGF protein level, or allowing determination of the efficacy of a given treatment regimen for an individual afflicted with such a disorder. The level of MFGF polypeptides may be measured from cells in bodily fluid, such as in blood samples.

Another application of anti-MFGF antibodies of the present invention is in the immunological screening of cDNA libraries constructed in expression vectors such as λgt11, λgt18-23, λZAP, and λORF8. Messenger libraries of this type, having coding sequences inserted in the correct reading frame and orientation, can produce fusion proteins. For instance, λgt11 will produce fusion proteins whose amino termini consist of γ-galactosidase amino acid sequences and whose carboxy termini consist of a foreign polypeptide. Antigenic epitopes of an MFGF protein, e.g., other orthologs of a particular MFGF protein or other paralogs from the same species, can then be detected with antibodies, as, for example, reacting nitrocellulose filters lifted from infected plates with anti-MFGF antibodies. Positive phage detected by this assay can then be isolated from the infected plate. Thus, the presence of MFGF homologs can be detected and cloned from other animals, as can alternate isoforms (including splice variants) from humans.

4.6. Transgenic Animals

The invention further provides for transgenic animals, which can be used for a variety of purposes, e.g., to identify MFGF therapeutics. Transgenic animals of the invention include non-human animals containing a heterologous MFGF gene or fragment thereof under the control of an MFGF promoter or under the control of a heterologous promoter. Accordingly, the transgenic animals of the invention can be animals expressing a transgene encoding a wild-type MFGF protein or fragment thereof or variants thereof, including mutants and polymorphic variants thereof Such animals can be used, e.g., to determine the effect of a difference in amino acid sequence of an MFGF protein from the sequence set forth in SEQ ID NOS. 2 or 5, such as a polymorphic difference. These animals can also be used to determine the effect of expression of an MFGF protein in a specific site or for identifying MFGF therapeutics or confirming their activity in vivo.

The transgenic animals can also be animals containing a transgene, such as reporter gene, under the control of an MFGF promoter or fragment thereof. These animals are useful, e.g., for identifying MFGF drugs that modulate production of MFGF, such as by modulating MFGF gene expression. An MFGF gene promoter can be isolated, e.g., by screening of a genomic library with an MFGF cDNA fragment and characterized according to methods known in the art. In a preferred embodiment of the present invention, the transgenic animal containing said MFGF reporter gene is used to screen a class of bioactive molecules known as steroid hormones for their ability to modulate MFGF expression. In a more preferred embodiment of the invention, the steroid hormones screened for MFGF expression modulating activity belong to the group known as androgens. In a still more preferred embodiment of the invention, the steroid hormone is testosterone or a testosterone analog. Yet other non-human animals within the scope of the invention include those in which the expression of the endogenous MFGF gene has been mutated or “knocked out”. A “knock out” animal is one carrying a homozygous or heterozygous deletion of a particular gene or genes. These animals could be useful to determine whether the absence of MFGF will result in a specific phenotype, in particular whether these mice have or are likely to develop a specific disease, such as high susceptibility to heart disease or cancer. Furthermore these animals are useful in screens for drugs which alleviate or attenuate the disease condition resulting from the mutation of the MFGF gene as outlined below. These animals are also useful for determining the effect of a specific amino acid difference, or allelic variation, in an MFGF gene. That is, the MFGF knock out animals can be crossed with transgenic animals expressing, e.g., a mutated form or allelic variant of MFGF, thus resulting in an animal which expresses only the mutated protein and not the wild-type MFGF protein. In a preferred embodiment of this aspect of the invention, a transgenic MFGF knock-out mouse, carrying the mutated MFGF locus on one or both of its chromosomes, is used as a model system for transgenic or drug treatment of the condition resulting from loss of MFGF expression.

Methods for obtaining transgenic and knockout non-human animals are well known in the art. Knock out mice are generated by homologous integration of a “knock out” construct into a mouse embryonic stem cell chromosome which encodes the gene to be knocked out. In one embodiment, gene targeting, which is a method of using homologous recombination to modify an animal's genome, can be used to introduce changes into cultured embryonic stem cells. By targeting a MFGF gene of interest in ES cells, these changes can be introduced into the germlines of animals to generate chimeras. The gene targeting procedure is accomplished by introducing into tissue culture cells a DNA targeting construct that includes a segment homologous to a target MFGF locus, and which also includes an intended sequence modification to the MFGF genomic sequence (e.g., insertion, deletion, point mutation). The treated cells are then screened for accurate targeting to identify and isolate those which have been properly targeted.

Gene targeting in embryonic stem cells is in fact a scheme contemplated by the present invention as a means for disrupting a MFGF gene function through the use of a targeting transgene construct designed to undergo homologous recombination with one or more MFGF genomic sequences. The targeting construct can be arranged so that, upon recombination with an element of a MFGF gene, a positive selection marker is inserted into (or replaces) coding sequences of the gene. The inserted sequence functionally disrupts the MFGF gene, while also providing a positive selection trait. Exemplary MFGF targeting constructs are described in more detail below.

Generally, the embryonic stem cells (ES cells) used to produce the knockout animals will be of the same species as the knockout animal to be generated. Thus for example, mouse embryonic stem cells will usually be used for generation of knockout mice.

Embryonic stem cells are generated and maintained using methods well known to the skilled artisan such as those described by Doetschman et al. (1985) J Embryol. Exp. MoMFGFhol. 87:2745). Any line of ES cells can be used, however, the line chosen is typically selected for the ability of the cells to integrate into and become part of the germ line of a developing embryo so as to create germ line transmission of the knockout construct. Thus, any ES cell line that is believed to have this capability is suitable for use herein. One mouse strain that is typically used for production of ES cells, is the 129J strain. Another ES cell line is murine cell line D3 (American Type Culture Collection, catalog no. CKL 1934) Still another preferred ES cell line is the WW6 cell line (ioffe et al. (1995) PNAS 92:7357-7361). The cells are cultured and prepared for knockout construct insertion using methods well known to the skilled artisan, such as those set forth by Robertson in: Teratocarcinomas and Embryonic Stem Cells: A Practical Approach, E. J. Robertson, ed. IRL Press, Washington, D.C. [1987]); by Bradley et al. (1986) Current Topics in Devel. Biol. 20:357-371); and by Hogan et al. (Manipulating the Mouse Embryo: A Laboratory Manual, Cold Spring Harbor Laboratory Press, Cold Spring Harbor, N.Y. [1986]).

A knock out construct refers to a uniquely configured fragment of nucleic acid which is introduced into a stem cell line and allowed to recombine with the genome at the chromosomal locus of the gene of interest to be mutated. Thus a given knock out construct is specific for a given gene to be targeted for disruption. Nonetheless, many common elements exist among these constructs and these elements are well known in the art A typical knock out construct contains nucleic acid fragments of not less than about 0.5 kb nor more than about 10.0 kb from both the 5 ′ and the 3′ ends of the genomic locus which encodes the gene to be mutated. These two fragments are separated by an intervening fragment of nucleic acid which encodes a positive selectable marker, such as the neomycin resistance gene (neo^{R}). The resulting nucleic acid fragment, consisting of a nucleic acid from the extreme 5′ end of the genomic locus linked to a nucleic acid encoding a positive selectable marker which is in turn linked to a nucleic acid from the extreme 3′ end of the genomic locus of interest, omits most of the coding sequence for MFGF or other gene of interest to be knocked out. When the resulting construct recombines homologously with the chromosome at this locus, it results in the loss of the omitted coding sequence, otherwise known as the structural gene, from the genomic locus. A stem cell in which such a rare homologous recombination event has taken place can be selected for by virtue of the stable integration into the genome of the nucleic acid of the gene encoding the positive selectable marker and subsequent selection for cells expressing this marker gene in the presence of an appropriate drug (neomycin in this example).

Variations on this basic technique also exist and are well known in the art. For example, a “knock-in” construct refers to the same basic arrangement of a nucleic acid encoding a 5′ genomic locus fragment linked to nucleic acid encoding a positive selectable marker which in turn is linked to a nucleic acid encoding a 3′ genomic locus fragment, but which differs in that none of the coding sequence is omitted and thus the 5′ and the 3′ genomic fragments used were initially contiguous before being disrupted by the introduction of the nucleic acid encoding the positive selectable marker gene. This “knock-in” type of construct is thus very useful for the construction of mutant transgenic animals when only a limited region of the genomic locus of the gene to be mutated, such as a single exon, is available for cloning and genetic manipulation. Alternatively, the “knock-in” construct can be used to specifically eliminate a single functional domain of the targetted gene, resulting in a transgenic animal which expresses a polypeptide of the targetted gene which is defective in one function, while retaining the function of other domains of the encoded polypeptide. This type of “knock-in” mutant frequently has the characteristic of a so-called “dominant negative” mutant because, especially in the case of proteins which homomultimerize, it can specifically block the action of (or “poison”) the polypeptide product of the wild-type gene from which it was derived. In a variation of the knock-in technique, a marker gene is integrated at the genomic locus of interest such that expression of the marker gene comes under the control of the transcriptional regulatory elements of the targeted gene. A marker gene is one that encodes an enzyme whose activity can be detected (e.g., b-galactosidase), the enzyme substrate can be added to the cells under suitable conditions, and the enzymatic activity can be analyzed One skilled in the art will be familiar with other useful markers and the means for detecting their presence in a given cell. All such markers are contemplated as being included within the scope of the teaching of this invention.

As mentioned above, the homologous recombination of the above described “knock out” and “knock in” constructs is very rare and frequently such a construct inserts nonhomologously into a random region of the genome where it has no effect on the gene which has been targeted for deletion, and where it can potentially recombine so as to disrupt another gene which was otherwise not intended to be altered. Such nonhomologous recombination events can be selected against by modifying the abovementioned knock out and knock in constructs so that they are flanked by negative selectable markers at either end (particularly through the use of two allelic variants of the thymidine kinase gene, the polypeptide product of which can be selected against in expressing cell lines in an appropriate tissue culture medium well known in the art—i.e. one containing a drug such as 5-bromodeoxyuridine). Thus a preferred embodiment of such a knock out or knock in construct of the invention consist of a nucleic acid encoding a negative selectable marker linked to a nucleic acid encoding a 5′ end of a genomic locus linked to a nucleic acid of a positive selectable marker which in turn is linked to a nucleic acid encoding a 3′ end of the same genomic locus which in turn is linked to a second nucleic acid encoding a negative selectable marker Nonhomologous recombination between the resulting knock out construct and the genome will usually result in the stable integration of one or both of these negative selectable marker genes and hence cells which have undergone nonhomologous recombination can be selected against by growth in the appropriate selective media (e.g. media containing a drug such as 5-bromodeoxyuridine for example). Simultaneous selection for the positive selectable marker and against the negative selectable marker will result in a vast enrichment for clones in which the knock out construct has recombined homologously at the locus of the gene intended to be mutated. The presence of the predicted chromosomal alteration at the targeted gene locus in the resulting knock out stem cell line can be confirmed by means of Southern blot analytical techniques which are well known to those familiar in the art. Alternatively, PCR can be used.

Each knockout construct to be inserted into the cell must first be in the linear form. Therefore, if the knockout construct has been inserted into a vector (described infra), linearization is accomplished by digesting the DNA with a suitable restriction endonuclease selected to cut only within the vector sequence and not within the knockout construct sequence.

For insertion, the knockout construct is added to the ES cells under appropriate conditions for the insertion method chosen, as is known to the skilled artisan. For example, if the ES cells are to be electroporated, the ES cells and knockout construct DNA are exposed to an electric pulse using an electroporation machine and following the manufacturer's guidelines for use. After electroporation, the ES cells are typically allowed to recover under suitable incubation conditions. The cells are then screened for the presence of the knock out construct as explained above. Where more than one construct is to be introduced into the ES cell, each knockout construct can be introduced simultaneously or one at a time.

After suitable ES cells containing the knockout construct in the proper location have been identified by the selection techniques outlined above, the cells can be inserted into an embryo. Insertion may be accomplished in a variety of ways known to the skilled artisan, however a preferred method is by microinjection. For microinjection, about 10-30 cells are collected into a micropipet and injected into embryos that are at the proper stage of development to permit integration of the foreign ES cell containing the knockout construct into the developing embryo. For instance, the transformed ES cells can be microinjected into blastocytes. The suitable stage of development for the embryo used for insertion of ES cells is very species dependent, however for mice it is about 3.5 days. The embryos are obtained by perfusing the uterus of pregnant females. Suitable methods for accomplishing this are known to the skilled artisan, and are set forth by, e.g., Bradley et al. (supra).

While any embryo of the right stage of development is suitable for use, preferred embryos are male. In mice, the preferred embryos also have genes coding for a coat color that is different from the coat color encoded by the ES cell genes. In this way, the offspring can be screened easily for the presence of the knockout construct by looking for mosaic coat color (indicating that the ES cell was incorporated into the developing embryo). Thus, for example, if the ES cell line carries the genes for white fur, the embryo selected will carry genes for black or brown fur.

After the ES cell has been introduced into the embryo, the embryo may be implanted into the uterus of a pseudopregnant foster mother for gestation. While any foster mother may be used, the foster mother is typically selected for her ability to breed and reproduce well, and for her ability to care for the young. Such foster mothers are typically prepared by mating with vasectomized males of the same species. The stage of the pseudopregnant foster mother is important for successful implantation, and it is species dependent. For mice, this stage is about 2-3 days pseudopregnant.

Offspring that are born to the foster mother may be screened initially for mosaic coat color where the coat color selection strategy (as described above, and in the appended examples) has been employed. In addition, or as an alternative, DNA from tail tissue of the offspring may be screened for the presence of the knockout construct using Southern blots and/or PCR as described above. Offspring that appear to be mosaics may then be crossed to each other, if they are believed to carry the knockout construct in their germ line, in order to generate homozygous knockout animals. Homozygotes may be identified by Southern blotting of equivalent amounts of genomic DNA from mice that are the product of this cross, as well as mice that are known heterozygotes and wild type mice.

Other means of identifying and characterizing the knockout offspring are available. For example, Northern blots can be used to probe the mRNA for the presence or absence of transcripts encoding either the gene knocked out, the marker gene, or both. In addition, Western blots can be used to assess the level of expression of the MFGF gene knocked out in various tissues of the offspring by probing the Western blot with an antibody against the particular MFGF protein, or an antibody against the marker gene product, where this gene is expressed. Finally, in situ analysis (such as fixing the cells and labeling with antibody) and/or FACS (fluorescence activated cell sorting) analysis of various cells from the offspring can be conducted using suitable antibodies to look for the presence or absence of the knockout construct gene product.

Yet other methods of making knock-out or disruption transgenic animals are also generally known. See, for example, Manipulating the Mouse Embryo, (Cold Spring Harbor Laboratory Press, Cold Spring Harbor, N.Y., 1986). Recombinase dependent knockouts can also be generated, e.g. by homologous recombination to insert target sequences, such that tissue specific and/or temporal control of inactivation of a MFGF-gene can be controlled by recombinase sequences (described infra).

Animals containing more than one knockout construct and/or more than one transgene expression construct are prepared in any of several ways. The preferred manner of preparation is to generate a series of mammals, each containing one of the desired transgenic phenotypes. Such animals are bred together through a series of crosses, backcrosses and selections, to ultimately generate a single animal containing all desired knockout constructs and/or expression constructs, where the animal is otherwise congenic (genetically identical) to the wild type except for the presence of the knockout construct(s) and/or transgene(s).

A MFGF transgene can encode the wild-type form of the protein, or can encode homologs thereof, including both agonists and antagonists, as well as antisense constructs. In preferred embodiments, the expression of the transgene is restricted to specific subsets of cells, tissues or developmental stages utilizing, for example, cis-acting sequences that control expression in the desired pattern. In the present invention, such mosaic expression of a MFGF protein can be essential for many forms of lineage analysis and can additionally provide a means to assess the effects of, for example, lack of MFGF expression which might grossly alter development in small patches of tissue within an otherwise normal embryo. Toward this and, tissue-specific regulatory sequences and conditional regulatory sequences can be used to control expression of the transgene in certain spatial patterns. Moreover, temporal patterns of expression can be provided by, for example, conditional recombination systems or prokaryotic transcriptional regulatory sequences.

Genetic techniques, which allow for the expression of transgenes can be regulated via site-specific genetic manipulation in vivo, are known to those skilled in the art. For instance, genetic systems are available which allow for the regulated expression of a recombinase that catalyzes the genetic recombination of a target sequence. As used herein, the phrase “target sequence” refers to a nucleotide sequence that is genetically recombined by a recombinase. The target sequence is flanked by recombinase recognition sequences and is generally either excised or inverted in cells expressing recombinase activity. Recombinase catalyzed recombination events can be designed such that recombination of the target sequence results in either the activation or repression of expression of one of the subject MFGF proteins. For example, excision of a target sequence which interferes with the expression of a recombinant MFGF gene, such as one which encodes an antagonistic homolog or an antisense transcript, can be designed to activate expression of that gene. This interference with expression of the protein can result from a variety of mechanisms, such as spatial separation of the MFGF gene from the promoter element or an internal stop codon. Moreover, the transgene can be made wherein the coding sequence of the gene is flanked by recombinase recognition sequences and is initially transfected into cells in a 3′ to 5′ orientation with respect to the promoter element. In such an instance, inversion of the target sequence will reorient the subject gene by placing the 5′ end of the coding sequence in an orientation with respect to the promoter element which allow for promoter driven transcriptional activation.

The transgenic animals of the present invention all include within a plurality of their cells a transgene of the present invention, which transgene alters the phenotype of the “host cell” with respect to regulation of cell growth, death and/or differentiation. Since it is possible to produce transgenic organisms of the invention utilizing one or more of the transgene constructs described herein, a general description will be given of the production of transgenic organisms by referring generally to exogenous genetic material. This general description can be adapted by those skilled in the art in order to incorporate specific transgene sequences into organisms utilizing the methods and materials described below.

In an illustrative embodiment, either the cre/loxP recombinase system of bacteriophage P1 (Lakso et al. (1992) PNAS 89:6232-6236; Orban et al. (1992) PNAS 89:6861-6865) or the FLP recombinase system of Saccharomyces cerevisiae (O'Gorman et al. (1991) Science 251:1351-1355; PCT publication WO 92/15694) can be used to generate in vivo site-specific genetic recombination systems. Cre recombinase catalyzes the site-specific recombination of an intervening target sequence located between loxP sequences. loxP sequences are 34 base pair nucleotide repeat sequences to-which the Cre recombinase binds and are required for Cre recombinase mediated genetic recombination. The orientation of loxP sequences determines whether the intervening target sequence is excised or inverted when Cre recombinase is present (Abremski et al. (1984) J Biol. Chem. 259:1509-1514); catalyzing the excision of the target sequence when the loxP sequences are oriented as direct repeats and catalyzes inversion of the target sequence when loxP sequences are oriented as inverted repeats.

Accordingly, genetic recombination of the target sequence is dependent on expression of the Cre recombinase. Expression of the recombinase can be regulated by promoter elements which are subject to regulatory control, e.g., tissue-specific, developmental stage-specific, inducible or repressible by externally added agents. This regulated control will result in genetic recombination of the target sequence only in cells where recombinase expression is mediated by the promoter element. Thus, the activation expression of a recombinant MFGF protein can be regulated via control of recombinase expression.

Use of the cre/loxP recombinase system to regulate expression of a recombinant MFGF protein requires the construction of a transgenic animal containing transgenes encoding both the Cre recombinase and the subject protein. Animals containing both the Cre recombinase and a recombinant MFGF gene can be provided through the construction of “double” transgenic animals. A convenient method for providing such animals is to mate two transgenic animals each containing a transgene, e.g., a MFGF gene and recombinase gene.

One advantage derived from initially constructing transgenic animals containing a MFGF transgene in a recombinase-mediated expressible format derives from the likelihood that the subject protein, whether agonistic or antagonistic, can be deleterious upon expression in the transgenic animal. In such an instance, a founder population, in which the subject transgene is silent in all tissues, can be propagated and maintained. Individuals of this founder population can be crossed with animals expressing the recombinase in, for example, one or more tissues and/or a desired temporal pattern. Thus, the creation of a founder population in which, for example, an antagonistic MFGF transgene is silent will allow the study of progeny from that founder in which disruption of MFGF mediated induction in a particular tissue or at certain developmental stages would result in, for example, a lethal phenotype.

Similar conditional transgenes can be provided using prokaryotic promoter sequences which require prokaryotic proteins to be simultaneous expressed in order to facilitate expression of the MFGF transgene. Exemplary promoters and the corresponding trans-activating prokaryotic proteins are given in U.S. Pat. No 4,833,080.

Moreover, expression of the conditional transgenes can be induced by gene therapy-like methods wherein a gene encoding the trans-activating protein, e.g. a recombinase or a prokaryotic protein, is delivered to the tissue and caused to be expressed, such as in a cell-type specific manner. By this method, a MFGFA transgene could remain silent into adulthood until “turned on” by the introduction of the trans-activator.

In an exemplary embodiment, the “transgenic non-human animals” of the invention are produced by introducing transgenes into the germline of the non-human animal. Embryonal target cells at various developmental stages can be used to introduce transgenes. Different methods are used depending on the stage of development of the embryonal target cell. The specific line(s) of any animal used to practice this invention are selected for general good health, good embryo yields, good pronuclear visibility in the embryo, and good reproductive fitness. In addition, the haplotype is a significant factor. For example, when transgenic mice are to be produced, strains such as C57BL/6 or FVB lines are often used (Jackson Laboratory, Bar Harbor, Me.). Preferred strains are those with H-2^{b}, H-2^{d} or H-2q haplotypes such as C57BL/6 or DBA/1. The line(s) used to practice this invention may themselves be transgenics, and/or may be knockouts (i.e., obtained from animals which have one or more genes partially or completely suppressed).

In one embodiment, the transgene construct is introduced into a single stage embryo. The zygote is the best target for micro-injection. In the mouse, the male pronucleus reaches the size of approximately 20 micrometers in diameter which allows reproducible injection of 1-2 pl of DNA solution. The use of zygotes as a target for gene transfer has a major advantage in that in most cases the injected DNA will be incorporated into the host gene before the first cleavage (Brinster et al. (1985) PNAS 82:44384442). As a consequence, all cells of the transgenic animal will carry the incorporated transgene. This will in general also be reflected in the efficient transmission of the transgene to offspring of the founder since 50% of the germ cells will harbor the transgene.

Normally, fertilized embryos are incubated in suitable media until the pronuclei appear. At about this time, the nucleotide sequence comprising the transgene is introduced into the female or male pronucleus as described below. In some species such as mice, the male pronucleus is preferred. It is most preferred that the exogenous genetic material be added to the male DNA complement of the zygote prior to its being processed by the ovum nucleus or the zygote female pronucleus. It is thought that the ovum nucleus or female pronucleus release molecules which affect the male DNA complement, perhaps by replacing the protamines of the male DNA with histones, thereby facilitating the combination of the female and male DNA complements to form the diploid zygote.

Thus, it is preferred that the exogenous genetic material be added to the male complement of DNA or any other complement of DNA prior to its being affected by the female pronucleus. For example, the exogenous genetic material is added to the early male pronucleus, as soon as possible after the formation of the male pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material can then be added to the ovum or the decondensed sperm could be added to the ovum with the transgene constructs being added as soon as possible thereafter.

Introduction of the transgene nucleotide sequence into the embryo may be accomplished by any means known in the art such as, for example, microinjection, electroporation, or lipofection. Following introduction of the transgene nucleotide sequence into the embryo, the embryo may be incubated in vitro for varying amounts of time, or reimplanted into the surrogate host, or both. In vitro incubation to maturity is within the scope of this invention. One common method in to incubate the embryos in vitro for about 1-7 days, depending on the species, and then reimplant them into the surrogate host.

For the purposes of this invention a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of the transgene constructs which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of the transgene construct, in order to insure that one copy is functional. As regards the present invention, there will often be an advantage to having more than one functioning copy of each of the inserted exogenous DNA sequences to enhance the phenotypic expression of the exogenous DNA sequences.

Any technique which allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgenic animals produced in accordance with the present invention will include exogenous genetic material. As set out above, the exogenous genetic material will, in certain embodiments, be a DNA sequence which results in the production of a MFGF protein (either agonistic or antagonistic), and antisense transcript, or a MFGF mutant. Further, in such embodiments the sequence will be attached to a transcriptional control element, e.g., a promoter, which preferably allows the expression of the transgene product in a specific type of cell.

Retroviral infection can also be used to introduce transgene into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

A third type of target cell for transgene introduction is the embryonal stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) PNAS 83: 9065-9069; and Robertson et al. (1986) Nature 322:445448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

4.7. Screening Assays for MFGF Therapeutics

The invention further provides screening methods for identifying MFGF therapeutics, e.g., for treating and/or preventing the development of diseases or conditions caused by, or contributed to by an abnormal MFGF activity or which can benefit from a modulation of an MFGF activity or protein level. Examples of such diseases, conditions or disorders include without limitation: cancer e.g., cancers involving the growth of steroid hormone-responsive tumors (e.g. breast, prostate, or testicular cancer); vascular diseases or disorders (e.g. thrombotic stroke, ischemic stroke, as well as peripheral vascular disease resulting from atherosclerotic and thrombotic processes); cardiac disorders (e.g., myocardial infarction, congestive heart failure, unstable angina and ishemic heart disease); cardiovascular system diseases and disorders (e.g. those resulting from hypertension, hypotension, cardiomyocyte hypertrophy and congestive heart failure) wound healing; limb regeneration; periodontal regeneration; aid in the acceptance of tissue transplants or bone grafts; skin aging; hair loss; muscle wasting conditions (e.g. cachexia) neurological damage or disease (e.g. that associated with Alzheimer's disease, Parkinson's disease, AIDS-related complex, or cerebral palsy); or other diseases conditions or disorders which result from aberrations or alterations of MFGF-dependent processes including: collateral growth and remodeling of cardiac blood vessels, angiogenesis, cellular transformation through autocrine or paracrine mechanisms, chemotactic stimulation of cells (e.g. endothelial), neurite outgrowth of neuronal precursor cell types (e.g. PC12 phaeochromoctoma), maintenance of neural physiology of mature neurons, proliferation of embryonic mesenchyme and limb-bud precursor tissue, mesoderm induction and other developmental processes, stimulation of collagenase and plasminogen activator secretion, tumor vascularization, as well as tumor invasion and metastasis. A MFGF therapeutic can be any type of compound, including a protein, a peptide, peptidomimetic, small molecule, and nucleic acid. A nucleic acid can be, e.g., a gene, an antisense nucleic acid, a ribozyme, or a triplex molecule. An MFGF therapeutic of the invention can be an agonist or an antagonist. Preferred MFGF agonists include MFGF proteins or derivatives thereof which mimic at least one MFGF activity, e.g., fibroblast growth factor receptor binding or heparin sulfate binding. Other preferred agonists include compounds which are capable of increasing the production of an MFGF protein in a cell, e.g., compounds capable of upregulating the expression of an MFGF gene, and compounds which are capable of enhancing an MFGF activity and/or the interaction of an MFGF protein with another molecule, such as a target peptide. Preferred MFGF antagonists include MFGF proteins which are dominant negative proteins, which, e.g., are capable of binding to fibroblast growth factor receptors, but not heparin sulfate. Other preferred antagonists include compounds which decrease or inhibit the production of an MFGF protein in a cell and compounds which are capable of downregulating expression of an MFGF gene, and compounds which are capable of downregulating an MFGF activity and/or interaction of an MFGF protein with another molecule. In another preferred embodiment, an MFGF antagonist is a modified form of a target peptide, which is capable of interacting with the FGFR binding domain of an MFGF protein, but which does not have biological activity, e.g., which is not itself a cell surface receptor.

The invention also provides screening methods for identifying MFGF therapeutics which are capable of binding to an MFGF protein, e.g., a wild-type MFGF protein or a mutated form of an MFGF protein, and thereby modulate the growth factor activity of MFGF or otherwise cause the degradation of MFGF. For example, such an MFGF therapeutic can be an antibody or derivative thereof which interacts specifically with an MFGF protein (either wild-type or mutated).

Thus, the invention provides screening methods for identifying MFGF agonist and antagonist compounds, comprising selecting compounds which are capable of interacting with an MFGF protein or with a molecule capable of interacting with an MFGF protein such as an FGF receptor and/or heparin sulfate and/or a compound which is capable of modulating the interaction of an MFGF protein with another molecule, such as a receptor and/or heparin sulfate. In general, a molecule which is capable of interacting with an MFGF protein is referred to herein as “MFGF binding partner”.

The compounds of the invention can be identified using various assays depending on the type of compound and activity of the compound that is desired. In addition, as described herein, the test compounds can be further tested in animal models. Set forth below are at least some assays that can be used for identifying MFGF therapeutics. It is within the skill of the art to design additional assays for identifying MFGF therapeutics.

4.7.1. Cell-free Assays

Cell-free assays can be used to identify compounds which are capable of interacting with an MFGF protein or binding partner, to thereby modify the activity of the MFGF protein or binding partner. Such a compound can, e.g., modify the structure of an MFGF protein or binding partner and thereby effect its activity. Cell-free assays can also be used to identify compounds which modulate the interaction between an MFGF protein and an MFGF binding partner, such as a target peptide. In a preferred embodiment, cell-free assays for identifying such compounds consist essentially in a reaction mixture containing an MFGF protein and a test compound or a library of test compounds in the presence or absence of a binding partner. A test compound can be, e.g., a derivative of an MFGF binding partner, e.g., a biologically inactive target peptide, or a small molecule.

Accordingly, one exemplary screening assay of the present invention includes the steps of contacting an MFGF protein or functional fragment thereof or an MFGF binding partner with a test compound or library of test compounds and detecting the formation of complexes. For detection purposes, the molecule can be labeled with a specific marker and the test compound or library of test compounds labeled with a different marker. Interaction of a test compound with an MFGF protein or fragment thereof or MFGF binding partner can then be detected by determining the level of the two labels after an incubation step and a washing step. The presence of two labels after the washing step is indicative of an interaction.

An interaction between molecules can also be identified by using real-time BIA (Biomolecular Interaction Analysis, Pharmacia Biosensor AB) which detects surface plasmon resonance (SPR), an optical phenomenon. Detection depends on changes in the mass concentration of macromolecules at the biospecific interface, and does not require any labeling of interactants. In one embodiment, a library of test compounds can be immobilized on a sensor surface, e.g., which forms one wall of a micro-flow cell. A solution containing the MFGF protein, functional fragment thereof, MFGF analog or MFGF binding partner is then flown continuously over the sensor surface. A change in the resonance angle as shown on a signal recording, indicates that an interaction has occurred. This technique is further described, e.g., in BIA technology Handbook by Pharmacia.

Another exemplary screening assay of the present invention includes the steps of (a) forming a reaction mixture including: (i) an MFGF polypeptide, (ii) an MFGF binding partner, and (iii) a test compound; and (b) detecting interaction of the MFGF and the MFGF binding protein. The MFGF polypeptide and MFGF binding partner can be produced recombinantly, purified from a source, e.g., plasma, or chemically synthesized, as described herein. A statistically significant change (potentiation or inhibition) in the interaction of the MFGF and MFGF binding protein in the presence of the test compound, relative to the interaction in the absence of the test compound, indicates a potential agonist (mimetic or potentiator) or antagonist (inhibitor) of MFGF bioactivity for the test compound. The compounds of this assay can be contacted simultaneously. Alternatively, an MFGF protein can first be contacted with a test compound for an appropriate amount of time, following which the MFGF binding partner is added to the reaction mixture. The efficacy of the compound can be assessed by generating dose response curves from data obtained using various concentrations of the test compound. Moreover, a control assay can also be performed to provide a baseline for comparison. In the control assay, isolated and purified MFGF polypeptide or binding partner is added to a composition containing the MFGF binding partner or MFGF polypeptide, and the formation of a complex is quantitated in the absence of the test compound.

Complex formation between an MFGF protein and an MFGF binding partner may be detected by a variety of techniques. Modulation of the formation of complexes can be quantitated using, for example, detectably labeled proteins such as radiolabeled, fluorescently labeled, or enzymatically labeled MFGF proteins or MFGF binding partners, by immunoassay, or by chromatographic detection.

Typically, it will be desirable to immobilize either MFGF or its binding partner to facilitate separation of complexes from uncomplexed forms of one or both of the proteins, as well as to accommodate automation of the assay. Binding of MFGF to an MFGF binding partner, can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase/MFGF (GST/MFGF) fusion proteins can be adsorbed onto glutathione sepharose beads (Sigma Chemical, St. Louis, Mo.) or glutathione derivatized microtitre plates, which are then combined with the MFGF binding partner, e.g. an ³⁵S labeled MFGF binding partner, and the test compound, and the mixture incubated under conditions conducive to complex formation, e.g. at physiological conditions for salt and pH, though slightly more stringent conditions may be desired. Following incubation, the beads are washed to remove any unbound label, and the matrix immobilized and radiolabel determined directly (e.g. beads placed in scintilant), or in the supernatant after the complexes are subsequently dissociated. Alternatively, the complexes can be dissociated from the matrix, separated by SDS-PAGE, and the level of MFGF protein or MFGF binding partner found in the bead fraction quantitated from the gel using standard electrophoretic techniques such as described in the appended examples.

Other techniques for immobilizing proteins on matrices are also available for use in the subject assay. For instance, either MFGF or its cognate binding partner can be immobilized utilizing conjugation of biotin and streptavidin. For instance, biotinylated MFGF molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with MFGF can be derivatized to the wells of the plate, and MFGF trapped in the wells by antibody conjugation. As above, preparations of an MFGF binding protein and a test compound are incubated in the MFGF presenting wells of the plate, and the amount of complex trapped in the well can be quantitated. Exemplary methods for detecting such complexes, in addition to those described above for the GST-immobilized complexes, include immunodetection of complexes using antibodies reactive with the MFGF binding partner, or which are reactive with MFGF protein and compete with the binding partner; as well as enzyme-linked assays which rely on detecting an enzymatic activity associated with the binding partner, either intrinsic or extrinsic activity. In the instance of the latter, the enzyme can be chemically conjugated or provided as a fusion protein with the MFGF binding partner. To illustrate, the MFGF binding partner can be chemically cross-linked or genetically fused with horseradish peroxidase, and the amount of polypeptide trapped in the complex can be assessed with a chromogenic substrate of the enzyme, e.g. 3,3′-diamino-benzadine terahydrochloride or 4-chloro-1-napthol. Likewise, a fusion protein comprising the polypeptide and glutathione-S-transferase can be provided, and complex formation quantitated by detecting the GST activity using 1-chloro-2,4-dinitrobenzene (Habig et al (1974) J Biol Chem 249:7130).

For processes which rely on immunodetection for quantitating one of the proteins trapped in the complex, antibodies against the protein, such as anti-MFGF antibodies, can be used. Alternatively, the protein to be detected in the complex can be “epitope tagged” in the form of a fusion protein which includes, in addition to the MFGF sequence, a second polypeptide for which antibodies are readily available (e.g. from commercial sources). For instance, the GST fusion proteins described above can also be used for quantification of binding using antibodies against the GST moiety. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc.) or the pEZZ-protein A system (Pharmacia, N.J.).

Cell-free assays can also be used to identify compounds which interact with an MFGF protein and modulate an activity of an MFGF protein. Accordingly, in one embodiment, an MFGF protein is contacted with a test compound and the catalytic activity of MFGF is monitored. In one embodiment, the abililty of MFGF to bind a target molecule is determined. The binding affinity of MFGF to a target molecule can be determined according to methods known in the art. Determination of the enzymatic activity of MFGF can be performed with the aid of the substrate furanacryloyl-L-phenylalanyl-glycyl-glycine (FAPGG) under conditions described in Holmquist et al. (1979) Anal. Biochem. 95:540 and in U.S. Pat. No. 5,259,045.

4.7.2. Cell Based Assays

In addition to cell-free assays, such as described above, MFGF proteins as provided by the present invention, facilitate the generation of cell-based assays, e.g., for identifying small molecule agonists or antagonists. In one embodiment, a cell expressing an MFGF receptor protein on the outer surface of its cellular membrane is incubated in the presence of a test compound alone or in the presence of a test compound and a MFGF protein and the interaction between the test compound and the MFGF receptor protein or between the MFGF protein (preferably a tagged MFGF protein) and the MFGF receptor is detected, e.g., by using a microphysiometer (McConnell et al. (1992) Science 257:1906). An interaction between the MFGF receptor protein and either the test compound or the MFGF protein is detected by the microphysiometer as a change in the acidification of the medium. This assay system thus provides a means of identifying molecular antagonists which, for example, function by interfering with MFGF-MFGF receptor interactions, as well as molecular agonist which, for example, function by activating an MFGF receptor.

Cell based assays can also be used to identify compounds which modulate expression of an MFGF gene, modulate translation of an MFGF mRNA, or which modulate the stability of an MFGF mRNA or protein. Accordingly, in one embodiment, a cell which is capable of producing MFGF, e.g., a cardiac myocyte, is incubated with a test compound and the amount of MFGF produced in the cell medium is measured and compared to that produced from a cell which has not been contacted with the test compound. The specificity of the compound vis a vis MFGF can be confirmed by various control analysis, e.g., measuring the expression of one or more control genes. Compounds which can be tested include small molecules, proteins, and nucleic acids. In particular, this assay can be used to determine the efficacy of MFGF antisense molecules or ribozymes.

In another embodiment, the effect of a test compound on transcription of an MFGF gene is determined by transfection experiments using a reporter gene operatively linked to at least a portion of the promoter of an MFGF gene. A promoter region of a gene can be isolated, e.g., from a genomic library according to methods known in the art. The reporter gene can be any gene encoding a protein which is readily quantifiable, e.g, the luciferase or CAT gene. Such reporter gene are well known in the art.

This invention further pertains to novel agents identified by the above-described screening assays and uses thereof for treatments as described herein.

4.8. Predictive Medicine

The invention further features predictive medicines, which are based, at least in part, on the identity of the novel MFGF genes and alterations in the genes and related pathway genes, which affect the expression level and/or function of the encoded MFGF protein in a subject.

For example, information obtained using the diagnostic assays described herein (alone or in conjunction with information on another genetic defect, which contributes to the same disease) is useful for diagnosing or confirming that a symptomatic subject (e.g. a subject symptomatic for congestive heart failure), has a genetic defect (e.g. in an MFGF gene or in a gene that regulates the expression of an MFGF gene), which causes or contributes to the particular disease or disorder. Alternatively, the information (alone or in conjunction with information on another genetic defect, which contributes to the same disease) can be used prognostically for predicting whether a non-symptomatic subject is likely to develop a disease or condition, which is caused by or contributed to by an abnormal MFGF activity or protein level in a subject. Based on the prognostic information, a doctor can recommend a regimen (e.g. diet or exercise) or therapeutic protocol, useful for preventing or prolonging onset of the particular disease or condition in the individual.

In addition, knowledge of the particular alteration or alterations, resulting in defective or deficient MFGF genes or proteins in an individual (the MFGF genetic profile), alone or in conjunction with information on other genetic defects contributing to the same disease (the genetic profile of the particular disease) allows customization of therapy for a particular disease to the individual's genetic profile, the goal of “pharmacogenomics”. For example, an individual's MFGF genetic profile or the genetic profile of a disease or condition, to which MFGF genetic alterations cause or contribute, can enable a doctor to 1) more effectively prescribe a drug that will address the molecular basis of the disease or condition; and 2) better determine the appropriate dosage of a particular drug. For example, the expression level of MFGF proteins, alone or in conjunction with the expression level of other genes, known to contribute to the same disease, can be measured in many patients at various stages of the disease to generate a transcriptional or expression profile of the disease. Expression patterns of individual patients can then be compared to the expression profile of the disease to determine the appropriate drug and dose to administer to the patient.

The ability to target populations expected to show the highest clinical benefit, based on the MFGF or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling (e.g. since the use of MFGF as a marker is useful for optimizing effective dose).

These and other methods are described in further detail in the following sections.

4.8.1. Prognostic and Diagnostic Assays

The present methods provide means for determining if a subject has (diagnostic) or is at risk of developing (prognostic) a disease, condition or disorder that is associated with an aberrant MFGF activity, e.g., an aberrant level of MFGF protein or an aberrant bioactivity. Examples of such diseases, conditions or disorders include without limitation: cancer e.g. cancers involving the growth of steroid hormone-responsive tumors (e.g. breast, prostate, or testicular cancer); vascular diseases or disorders (e.g. thrombotic stroke, ischemic stroke, as well as peripheral vascular disease resulting from atherosclerotic and thrombotic processes); cardiac disorders (e.g. myocardial infarction, unstable angina and ishemic heart disease); cardiovascular system diseases and disorders (e.g. those resulting from hypertension, hypotension, cardiomyocyte hypertrophy and congestive heart failure) wound healing; limb regeneration; neurological damage or disease (e.g. that associated with Alzheimer's disease, Parkinson's disease, AIDS-related complex, or cerebral palsy); or other diseases conditions or disorders which result from aberrations or alterations of MFGF-dependent processes including: collateral growth and remodeling of cardiac blood vessels, angiogenesis, cellular transformation through autocrine or paracrine mechanisms, chemotactic stimulation of cells (e.g. endothelial), neurite outgrowth of neuronal precursor cell types (e.g. PC12 phaeochromoctoma), maintenance of neural physiology of mature neurons, proliferation of embryonic mesenchyme and limb-bud precursor tissue, mesoderm induction and other developmental processes, stimulation of collagenase and plasminogen activator secretion, tumor vascularization, as well as tumor invasion and metastasis.

Accordingly, the invention provides methods for determining whether a subject has or is likely to develop, a disease or condition that is caused by or contributed to by an abnormal MFGF level or bioactivity, for example, comprising determining the level of an MFGF gene or protein, an MFGF bioactivity and/or the presence of a mutation or particular polymorphic variant in the MFGF gene.

In one embodiment, the method comprises determining whether a subject has an abnormal mRNA and/or protein level of MFGF, such as by Northern blot analysis, reverse transcription-polymerase chain reaction (RT-PCR), in situ hybridization, immunoprecipitation, Western blot hybridization, or immunohistochemistry. According to the method, cells are obtained from a subject and the MFGF protein or mRNA level is determined and compared to the level of MFGF protein or mRNA level in a healthy subject. An abnormal level of MFGF polypeptide or mRNA level is likely to be indicative of an aberrant MFGF activity.

In another embodiment, the method comprises measuring at least one activity of MFGF. For example, the affinity of MFGF for heparin, can be determined, e.g., as described herein. Similarly, the constant of affinity of an MFGF protein of a subject with a binding partner (e.g. FGF receptor) can be determined. Comparison of the results obtained with results from similar analysis performed on MFGF proteins from healthy subjects is indicative of whether a subject has an abnormal MFGF activity.

In preferred embodiments, the methods for determining whether a subject has or is at risk for developing a disease, which is caused by or contributed to by an aberrant MFGF activity is characterized as comprising detecting, in a sample of cells from the subject, the presence or absence of a genetic alteration characterized by at least one of (i) an alteration affecting the integrity of a gene encoding an MFGF polypeptide, or (ii) the mis-expression of the MFGF gene. For example, such genetic alterations can be detected by ascertaining the existence of at least one of (i) a deletion of one or more nucleotides from an MFGF gene, (ii) an addition of one or more nucleotides to an MFGF gene, (iii) a substitution of one or more nucleotides of an MFGF gene, (iv) a gross chromosomal rearrangement of an MFGF gene, (v) a gross alteration in the level of a messenger RNA transcript of an MFGF gene, (vii) aberrant modification of an MFGF gene, such as of the methylation pattern of the genomic DNA, (vii) the presence of a non-wild type splicing pattern of a messenger RNA transcript of an MFGF gene, (viii) a non-wild type level of an MFGF polypeptide, (ix) allelic loss of an MFGF gene, and/or (x) inappropriate post-translational modification of an MFGF polypeptide. As set out below, the present invention provides a large number of assay techniques for detecting alterations in an MFGF gene. These methods include, but are not limited to, methods involving sequence analysis, Southern blot hybridization, restriction enzyme site mapping, and methods involving detection of absence of nucleotide pairing between the nucleic acid to be analyzed and a probe. These and other methods are further described infra.

Specific diseases or disorders, e.g., genetic diseases or disorders, are associated with specific allelic variants of polymorphic regions of certain genes, which do not necessarily encode a mutated protein. Thus, the presence of a specific allelic variant of a polymorphic region of a gene, such as a single nucleotide polymorphism (“SNP”), in a subject can render the subject susceptible to developing a specific disease or disorder. Polymorphic regions in genes, e.g, MFGF genes, can be identified, by determining the nucleotide sequence of genes in populations of individuals. If a polymorphic region, e.g., SNP is identified, then the link with a specific disease can be determined by studying specific populations of individuals, e.g, individuals which developed a specific disease, such as congestive heart failure, hypertension, hypotension, or a cancer (e.g. a cancer involving growth of a steroid responsive tumor or tumors). A polymorphic region can be located in any region of a gene, e.g., exons, in coding or non coding regions of exons, introns, and promoter region.

It is likely that MFGF genes comprise polymorphic regions, specific alleles of which may be associated with specific diseases or conditions or with an increased likelihood of developing such diseases or conditions. Thus, the invention provides methods for determining the identity of the allele or allelic variant of a polymorphic region of an MFGF gene in a subject, to thereby determine whether the subject has or is at risk of developing a disease or disorder associated with a specific allelic variant of a polymorphic region.

In an exemplary embodiment, there is provided a nucleic acid composition comprising a nucleic acid probe including a region of nucleotide sequence which is capable of hybridizing to a sense or antisense sequence of an MFGF gene or naturally occurring mutants thereof or 5′ or 3′ flanking sequences or intronic sequences naturally associated with the subject MFGF genes or naturally occurring mutants thereof The nucleic acid of a cell is rendered accessible for hybridization, the probe is contacted with the nucleic acid of the sample, and the hybridization of the probe to the sample nucleic acid is detected. Such techniques can be used to detect alterations or allelic variants at either the genomic or mRNA level, including deletions, substitutions, etc., as well as to determine mRNA transcript levels.

A preferred detection method is allele specific hybridization using probes overlapping the mutation or polymorphic site and having about 5, 10, 20, 25, or 30 nucleotides around the mutation or polymorphic region. In a preferred embodiment of the invention, several probes capable of hybridizing specifically to allelic variants, such as single nucleotide polymorphisms, are attached to a solid phase support, e.g., a “chip”. Oligonucleotides can be bound to a solid support by a variety of processes, including lithography. For example a chip can hold up to 250,000 oligonucleotides. Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

In certain embodiments, detection of the alteration comprises utilizing the probe/primer in a polymerase chain reaction (PCR) (see, e.g. U.S. Pat. Nos. 4,683,195 and 4,683,202), such as anchor PCR or RACE PCR, or, alternatively, in a ligase chain reaction (LCR) (see, e.g., Landegran et al. (1988) Science 241:1077-1080; and Nakazawa et al. (1994) PNAS 91:360-364), the latter of which can be particularly useful for detecting point mutations in the MFGF gene (see Abravaya et al. (1995) Nuc Acid Res 23:675-682). In a merely illustrative embodiment, the method includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize to an MFGF gene under conditions such that hybridization and amplification of the MFGF gene (if present) occurs, and (iv) detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), Q-Beta Replicase (Lizardi, P. M. et al., 1988, Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In a preferred embodiment of the subject assay, mutations in, or allelic variants, of an MFGF gene from a sample cell are identified by alterations in restriction enzyme cleavage patterns. For example, sample and control DNA is isolated, amplified (optionally), digested with one or more restriction endonucleases, and fragment length sizes are determined by gel electrophoresis. Moreover, the use of sequence specific ribozymes (see, for example, U.S. Pat. No. 5,498,531) can be used to score for the presence of specific mutations by development or loss of a ribozyme cleavage site.

In yet another embodiment, any of a variety of sequencing reactions known in the art can be used to directly sequence the MFGF gene and detect mutations by comparing the sequence of the sample MFGF with the corresponding wild-type (control) sequence. Exemplary sequencing reactions include those based on techniques developed by Maxim and Gilbert (Proc. Natl Acad Sci USA (1977) 74:560) or Sanger (Sanger et al (1977) Proc. Nat. Acad. Sci 74:5463). It is also contemplated that any of a variety of automated sequencing procedures may be utilized when performing the subject assays (Biotechniques (1995) 19:448), including sequencing by mass spectrometry (see, for example PCT publication WO 94/16101; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleic acid is detected, can be carried out.

In a further embodiment, protection from cleavage agents (such as a nuclease, hydroxylamine or osmium tetroxide and with piperidine) can be used to detect mismatched bases in RNA/RNA or RNA/DNA or DNA/DNA heteroduplexes (Myers, et al. (1985) Science 230:1242). In general, the art technique of “mismatch cleavage” starts by providing heteroduplexes formed by hybridizing (labelled) RNA or DNA containing the wild-type MFGF sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl Acad Sci USA 85:4397; Saleeba et al (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in MFGF cDNAs obtained from samples of cells. For example, the mut Y enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an MFGF sequence, e.g., a wild-type MFGF sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations or the identity of the allelic variant of a polymorphic region in MFGF genes. For example, single strand conformation polymorphism (SSCP) may be used to detect differences in electrophoretic mobility between mutant and wild type nucleic acids (Orita et al. (1989) Proc Natl. Acad Sci USA 86:2766, see also Cotton (1993) Mutat Res 285:125-144; and Hayashi (1992) Genet Anal Tech Appl 9:73-79). Single-stranded DNA fragments of sample and control MFGF nucleic acids are denatured and allowed to renature. The secondary structure of single-stranded nucleic acids varies according to sequence, the resulting alteration in electrophoretic mobility enables the detection of even a single base change. The DNA fragments may be labelled or detected with labelled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of mutant or wild-type fragments in polyacrylamide gels containing a gradient of denaturant is assayed using denaturing gradient gel electrophoresis (DGGE) (Myers et al (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:12753).

Examples of other techniques for detecting point mutations or the identity of the allelic variant of a polymorphic region include, but are not limited to, selective oligonucleotide hybridization, selective amplification, or selective primer extension. For example, oligonucleotide primers may be prepared in which the known mutation or nucleotide difference (e.g., in allelic variants) is placed centrally and then hybridized to target DNA under conditions which permit hybridization only if a perfect match is found (Saiki et al. (1986) Nature 324:163); Saiki et al (1989) Proc. Natl Acad. Sci USA 86:6230). Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labelled target DNA Alternatively, allele specific amplification technology which depends on selective PCR amplification may be used in conjunction with the instant invention. Oligonucleotides used as primers for specific amplification may carry the mutation or polymorphic region of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238. In addition it may be desirable to introduce a novel restriction site in the region of the mutation to create cleavage-based detection (Gasparini et al (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

In another embodiment, identification of the allelic variant is carried out using an oligonucleotide ligation assay (OLA), as described, e.g., in U.S. Pat. No. 4,998,617 and in Landegren, U. et al., Science 241:1077-1080 (1988). The OLA protocol uses two oligonucleotides which are designed to be capable of hybridizing to abutting sequences of a single strand of a target. One of the oligonucleotides is linked to a separation marker, e.g,. biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., Proc. Natl. Acad. Sci. (U.S.A.) 87:8923-8927 (1990). In this method, PCR is used to achieve the exponential amplification of target DNA, which is then detected using OLA.

Several techniques based on this OLA method have been developed and can be used to detect specific allelic variants of a polymorphic region of an MFGF gene. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

The invention further provides methods for detecting single nucleotide polymorphisms in an MFGF gene. Because single nucleotide polymorphisms constitute sites of variation flanked by regions of invariant sequence, their analysis requires no more than the determination of the identity of the single nucleotide present at the site of variation and it is unnecessary to determine a complete gene sequence for each patient. Several methods have been developed to facilitate the analysis of such single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA ™ is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A. -C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA9:107-112 (1992); Nyren, P. et al., Anal. Biochem. 208:171-175 (1993)). These methods differ from GBA TM in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. -C., et al., Amer. J. Hum. Genet. 52:46-59 (1993)).

For mutations that produce premature termination of protein translation, the protein truncation test (PTT) offers an efficient diagnostic approach (Roest, et. al., (1993) Hum. Mol. Genet. 2:1719-21; van der Luijt, et. al., (1994) Genomics 20:1-4). For PTT, RNA is initially isolated from available tissue and reverse-transcribed, and the segment of interest is amplified by PCR. The products of reverse transcription PCR are then used as a template for nested PCR amplification with a primer that contains an RNA polymerase promoter and a sequence for initiating eukaryotic translation. After amplification of the region of interest, the unique motifs incorporated into the primer permit sequential in vitro transcription and translation of the PCR products. Upon sodium dodecyl sulfate-polyacrylamide gel electrophoresis of translation products, the appearance of truncated polypeptides signals the presence of a mutation that causes premature termination of translation. In a variation of this technique, DNA (as opposed to RNA) is used as a PCR template when the target region of interest is derived from a single exon.

The methods described herein may be performed, for example, by utilizing pre-packaged diagnostic kits comprising at least one probe nucleic acid, primer set; and/or antibody reagent described herein, which may be conveniently used, e.g., in clinical settings to diagnose patients exhibiting symptoms or family history of a disease or illness involving an MFGF polypeptide.

Any cell type or tissue may be utilized in the diagnostics described below. In a preferred embodiment a bodily fluid, e.g., blood, is obtained from the subject to determine the presence of a mutation or the identity of the allelic variant of a polymorphic region of an MFGF gene. A bodily fluid, e.g, blood, can be obtained by known techniques (e.g. venipuncture). Alternatively, nucleic acid tests can be performed on dry samples (e.g. hair or skin). For prenatal diagnosis, fetal nucleic acid samples can be obtained from maternal blood as described in International Patent Application No. WO91/07660 to Bianchi. Alternatively, amniocytes or chorionic villi may be obtained for performing prenatal testing.

When using RNA or protein to determine the presence of a mutation or of a specific allelic variant of a polymorphic region of an MFGF gene, the cells or tissues that may be utilized must express the MFGF gene. Preferred cells for use in these methods include cardiac cells (see Examples). Alternative cells or tissues that can be used, can be identified by determining the expression pattern of the specific MFGF gene in a subject, such as by Northern blot analysis.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

Antibodies directed against wild type or mutant MFGF polypeptides or allelic variants thereof, which are discussed above, may also be used in disease diagnostics and prognostics. Such diagnostic methods, may be used to detect abnormalities in the level of MFGF polypeptide expression, or abnormalities in the structure and/or tissue, cellular, or subcellular location of an MFGF polypeptide. Structural differences may include, for example, differences in the size, electronegativity, or antigenicity of the mutant MFGF polypeptide relative to the normal MFGF polypeptide. Protein from the tissue or cell type to be analyzed may easily be detected or isolated using techniques which are well known to one of skill in the art, including but not limited to western blot analysis. For a detailed explanation of methods for carrying out Western blot analysis, see Sambrook et al, 1989, supra, at Chapter 18. The protein detection and isolation methods employed herein may also be such as those described in Harlow and Lane, for example, (Harlow, E. and Lane, D., 1988, “Antibodies: A Laboratory Manual”, Cold Spring Harbor Laboratory Press, Cold Spring Harbor, N.Y.), which is incorporated herein by reference in its entirety.

This can be accomplished, for example, by immunofluorescence techniques employing a fluorescently labeled antibody (see below) coupled with light microscopic, flow cytometric, or fluorimetric detection. The antibodies (or fragments thereof) useful in the present invention may, additionally, be employed histologically, as in immunofluorescence or immunoelectron microscopy, for in situ detection of MFGF polypeptides. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the MFGF polypeptide, but also its distribution in the examined tissue. Using the present invention, one of ordinary skill will readily perceive that any of a wide variety of histological methods (such as staining procedures) can be modified in order to achieve such in situ detection.

Often a solid phase support or carrier is used as a support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

One means for labeling an anti-MFGF polypeptide specific antibody is via linkage to an enzyme and use in an enzyme immunoassay (EIA) (Voller, “The Enzyme Linked Immunosorbent Assay (ELISA)”, Diagnostic Horizons 2:1-7, 1978, Microbiological Associates Quarterly Publication, Walkersville, Md.; Voller, et al., J. Clin. Pathol. 31:507-520 (1978); Butler, Meth. Enzymol. 73:482-523 (1981); Maggio, (ed.) Enzyme Immunoassay, CRC Press, Boca Raton, Fla., 1980; Ishikawa, et al., (eds.) Enzyme Immunoassay, Kgaku Shoin, Tokyo, 1981). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect fingerprint gene wild type or mutant peptides through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Moreover, it will be understood that any of the above methods for detecting alterations in a gene or gene product or polymorphic variants can be used to monitor the course of treatment or therapy.

4.8.2. Pharmacogenomics

Knowledge of the particular alteration or alterations, resulting in defective or deficient MFGF genes or proteins in an individual (the MFGF genetic profile), alone or in conjunction with information on other genetic defects contributing to the same disease (the genetic profile of the particular disease) allows a customization of the therapy for a particular disease to the individual's genetic profile, the goal of “pharmacogenomics”. For example, subjects having a specific allele of an MFGF gene may or may not exhibit symptoms of a particular disease or be predisposed of developing symptoms of a particular disease. Further, if those subjects are symptomatic, they may or may not respond to a certain drug, e.g., a specific MFGF therapeutic, but may respond to another. Thus, generation of an MFGF genetic profile, (e.g., categorization of alterations in MFGF genes which are associated with the development of a particular disease), from a population of subjects, who are symptomatic for a disease or condition that is caused by or contributed to by a defective and/or deficient MFGF gene and/or protein (an MFGF genetic population profile) and comparison of an individual's MFGF profile to the population profile, permits the selection or design of drugs that are expected to be safe and efficacious for a particular patient or patient population (i.e., a group of patients having the same genetic alteration).

For example, an MFGF population profile can be performed, by determining the MFGF profile, e.g., the identity of MFGF genes, in a patient population having a disease, which is caused by or contributed to by a defective or deficient MFGF gene. Optionally, the MFGF population profile can further include information relating to the response of the population to an MFGF therapeutic, using any of a variety of methods, including, monitoring: 1) the severity of symptoms associated with the MFGF related disease, 2) MFGF gene expression level, 3) MFGF mRNA level, and/or 4) MFGF protein level and (iii) dividing or categorizing the population based on the particular genetic alteration or alterations present in its MFGF gene or an MFGF pathway gene. The MFGF genetic population profile can also, optionally, indicate those particular alterations in which the patient was either responsive or non-responsive to a particular therapeutic. This information or population profile, is then useful for predicting which individuals should respond to particular drugs, based on their individual MFGF profile.

In a preferred embodiment, the MFGF profile is a transcriptional or expression level profile and step (i) is comprised of determining the expression level of MFGF proteins, alone or in conjunction with the expression level of other genes, known to contribute to the same disease. The MFGF profile can be measured in many patients at various stages of the disease.

Pharmacogenomic studies can also be performed using transgenic animals. For example, one can produce transgenic mice, e.g., as described herein, which contain a specific allelic variant of an MFGF gene. These mice can be created, e.g, by replacing their wild-type MFGF gene with an allele of the human MFGF gene. The response of these mice to specific MFGF therapeutics can then be determined.

4.8.3. Monitoring of Effects of MFGF Therapeutics During Clinical Trials

The ability to target populations expected to show the highest clinical benefit, based on the MFGF or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling (e.g. since the use of MFGF as a marker is useful for optimizing effective dose).

The treatment of an individual with an MFGF therapeutic can be monitored by determining MFGF characteristics, such as MFGF protein level or activity, MFGF mRNA level, and/or MFGF transcriptional level. This measurements will indicate whether the treatment is effective or whether it should be adjusted or optimized. Thus, MFGF can be used as a marker for the efficacy of a drug during clinical trials.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a preadministration sample from a subject prior to administration of the agent; (ii) detecting the level of expression of an MFGF protein, mRNA, or genomic DNA in the preadministration sample; (iii) obtaining one or more post-administration samples from the subject; (iv) detecting the level of expression or activity of the MFGF protein, mRNA, or genomic DNA in the post-administration samples; (v) comparing the level of expression or activity of the MFGF protein, mRNA, or genomic DNA in the preadministration sample with the MFGF protein, mRNA, or genomic DNA in the post administration sample or samples; and (vi) altering the administration of the agent to the subject accordingly. For example, increased administration of the agent may be desirable to increase the expression or activity of MFGF to higher levels than detected, i.e., to increase the effectiveness of the agent. Alternatively, decreased administration of the agent may be desirable to decrease expression or activity of MFGF to lower levels than detected, i.e., to decrease the effectiveness of the agent.

Cells of a subject may also be obtained before and after administration of an MFGF therapeutic to detect the level of expression of genes other than MFGF, to verify that the MFGF therapeutic does not increase or decrease the expression of genes which could be deleterious. This can be done, e.g., by using the method of transcriptional profiling. Thus, mRNA from cells exposed in vivo to an MFGF therapeutic and mRNA from the same type of cells that were not exposed to the MFGF therapeutic could be reverse transcribed and hybridized to a chip containing DNA from numerous genes, to thereby compare the expression of genes in cells treated and not treated with an MFGF-therapeutic. If, for example an MFGF therapeutic turns on the expression of a proto-oncogene in an individual, use of this particular MFGF therapeutic may be undesirable.

4.9. Methods of Treatment

The present invention provides for both prophylactic and therapeutic methods of treating a subject having or likely to develop a disorder associated with aberrant MFGF expression or activity, e.g., cardiac disorders or cancers.

4.9.1. Prophylactic Methods

In one aspect, the invention provides a method for preventing in a subject, a disease or condition associated with an aberrant MFGF expression or activity by administering to the subject an agent which modulates MFGF expression or at least one MFGF activity. Subjects at risk for such a disease can be identified by a diagnostic or prognostic assay, e.g., as described herein. Administration of a prophylactic agent can occur prior to the manifestation of symptoms characteristic of the MFGF aberrancy, such that a disease or disorder is prevented or, alternatively, delayed in its progression. Depending on the type of MFGF aberrancy, for example, a MFGF agonist or MFGF antagonist agent can be used for treating the subject prophylactically. The prophylactic methods are similar to therapeutic methods of the present invention and are further discussed in the following subsections.

4.9.2. Therapeutic Methods

In general the invention provides methods for treating a disease or condition which is caused by or contributed to by an aberrant MFGF activity comprising administering to the subject an effective amount of a compound which is capable of modulating an MFGF activity. Among the approaches which may be used to ameliorate disease symptoms involving an aberrant MFGF activity are, for example, antisense, ribozyme, and triple helix molecules described above. Examples of suitable compounds include the antagonists, agonists or homologues described in detail herein.

4.9.3. Diseases or Conditions that can be Treated or Prevented with MFGF Therapeutics

FGF proteins are generally potent mitogens for a variety of cells of mesodermal, ectodermal and endodermal origin including such cell types as fibroblasts, corneal and vascular endothelial cells and granulocytes. Indeed, the FGF family possesses many important physiological and homeostatic bioactivities. Not surprisingly, altered and/or aberrant expression of growth factors in general, and fibroblast growth factors in particular, has been associated with a number of disease processes including cardiac, vascular, and oncogenic conditions. The present invention provides MFGF therapeutics useful in the treatment of these as well as other diseases and disorders as enumerated below.

Adult cardiac myocytes have lost the capability to divide. Nonetheless, these cells express multiple growth factors and growth factor receptors and these receptors likely mediate normal functions as well as pathological conditions involving the heart. For example, FGF-1 expression has been shown to be greatly increased in viable cardiomyocytes close to small necrotic myocardial areas. Thus FGF-1 is thought to play a specific physiological role in a complex cascade leading to collateral growth and remodeling in response to ischemia. Furthermore, FGF-2 and other growth factors are thought to be involved in processes leading to cardiac hypertrophy. In particular, Cummins et al. have shown that FGF-2 mediates at least some changes in gene expression under conditions such as ischemia or volume overload that lead to adult cardiomyocyte hypertrophy (Cummins et al. (1993) Cardiovascular Research 27:1150-1154). Thus it is likely that FGF therapeutic agents, including molecular agonists and antagonists of FGF bioactivities, will be useful for treating any of a number of abnormal conditions of the heart. Indeed, FGF-2 has been shown to reduce myocardial infarction size after temporary coronary occlusion (Horrigan, et. al. (1996) Circulation 94:1927-1933).

As shown in the examples, Northern blot analysis has revealed that MFGF expression in cardiac tissue is particularly significant. Thus it is likely that MFGF therapeutic agents, including molecular agonists and antagonists of MFGF bioactivities, will be useful in treating a number of abnormal conditions of the heart. In a preferred embodiment, the compounds of the present invention are useful for regulating cardiac disease and in particular preventing or reversing the processes of congestive heart failure, myocardial infarction, cardiac ischemia, cardiomyocyte hypertrophy, and arterial hypertension. In fact, based on the apparent prominent expression of MFGF in the heart, and the significant nucleotide and amino acid sequence homology of certain active domains of MFGF with active domains of known FGF family members, MFGF is likely to promote healing of cardiac damage resulting from cardiac ischemia by promoting the division of cardiac myocytes as well as angiogenesis in adult myocardium recovering from ischemic injury. Furthermore, the polypeptides of the present invention, as a result of the ability to stimulate vascular endothelial cell growth, may also be more generally employed in treatment for stimulating revascularization of ischemic tissues due to any of a number of disease conditions such as thrombosis, arteriosclerosis, and other cardiovascular conditions.

MFGF antagonists are particularly useful for treating subjects with cardiac myocyte hypertrophy leading to enlargement of the heart. Thus, administration of a MFGF antagonist to such a subject will disrupt MFGF-dependent myocyte hypertrophy thereby blocking the physiological pathway leading to production of enlargement of the heart.

MFGF agonists are particularly useful for treating subjects who experience cardiac ischemia leading to a myocardial infarction. Since the binding of MFGF to a MFGF receptor promotes mitogenic and chemotactic effects on various cell types, administration of a MFGF therapeutic polypeptide or a MFGF agonist should stimulate cardiac myocyte cell growth as well as angiogenesis, thereby treating the subject's myocardial infarction resulting from ischemia.

MFGF therapeutics should also prove to be effective for treating unstable angina, a condition which can arise by coronary thrombosis leading to increased coronary obstruction and subsequent myocardial ischemia (Arbustini, et al. (1995) Am. J. Cardiol. 75:675-82) and which can ultimately progress to myocardial infarction and associated advanced myocardial ischemia. Immunohistochemical studies have demonstrated the specific accumulation of a fibroblast growth factor surrounding cardiomyocytes close to small necrotic tissue patches (Bernotat-Danielowski, S. et al. (1993) Cardiovascular Research 27:1220-8). Studies have also demonstrated that significantly elevated pericardial levels of growth factors such as basic fibroblast growth factor (FGF-2) are associated with unstable angina (Fujita, et al. (1996) Circulation 610-13). In addition, serum FGF-2 levels were found to be elevated in patients with ischemic heart disease, particularly in those with minimal coronary artery disease (Hasdai et al. (1997) International Journal of Cardiology 59:133-8). Furthermore, another factor, vascular endothelial growth factor, has been shown to be induced in hypoxic rat cardiac myocytes (Levy, et al. (1995) 76:758-66). These observations implicate secreted growth factor synthesis as an immediate early homeostatic response to ischemic heart disease processes and suggest that levels of such factors provide a sensitive prognostic and diagnostic indicator of ischemic heart disease. Based on structural and functional similarities with other fibroblast growth factors which are known to be involved in early stages of ischemic heart disease, MFGF and MFGF therapueutics are therefore likely to be effective in treating the above described conditions associated with ischemic heart disease.

Furthermore, based on the reported variation in angiogenic properties of other fibroblast growth factors discussed in detail below, MFGF therapeutics should prove generally useful for treating cardiac disease and its associated disorders including acute myocardial infarction. For example, fibroblast growth factors have been shown to stimulate the generation of angioblasts form mesoderm (Folkman and D'Amore (1996) Cell 87:1153-5). Fibroblast growth factors also stimulate many of the processes involved in the formation of new capillaries by endothelial cells including the destruction of capillary basement membrane, and endothelial cell migration, division and then reformation into capillary structures (Folkman and Klagsbrum (1987) Science 235:752-55). Specifically, fibroblast growth factors appear to stimulate these angiogenic processes through a multitude of specific biological activities including: chemotactic stimulation of endothelial cells (Terranova et al. (1985) J. Cell. Biol. 101:2330-4), direct stimulation of endothelial cell migration (Sato et al. (1988) J. Cell. Biol. 107:1199-205), indirect activation of interstitial collagenase through stimulation of tissue plasminogen activator and subsequent conversion of plasminogen into plasmin (the proteolytic activator of the collagenase proenzyme) (Monsenato et al. (1986) Proc. Natl. Acad. Sci. U.S.A. 83:7297-301), and upregulation of cell surface receptor expression such as the integrins leading to increased cell adherence to extracellular matrix proteins (Klein et al. (1993). These multiple activities account for the documented ability of fibroblast growth factors to stimulate ingrowth of new blood vessels in a rabbit corneal model system (Folkman and Klagsbrum (1987) Science 235:752-55). Furthermore the angiogenic therapeutic potential of fibroblast growth factors has been demonstrated in a study in which exogenous administration of FGF-1 was shown to enhance development of collateral blood flow in dogs with myocardial ischemia secondary to single-vessel coronary occlusion (Unger, et al. (1994) Am. J. Physiol. 266:H1588-95). Furthermore, short-term treatment with FGF-2 enhanced collateral development without increasing neointimal accumulation at sites of vascular injury (Lazarous, et al. (1996) Circulation 94:1074-82). Another study has concluded that delivery of a fibroblast growth factor to the pericardial cavity stimulated cardiac angiognesis and associated myocardial salvage thus providing a selective therapeutic and preventive modality of myocardial infarction (Uchida, et al. (1995) Am Heart J. 130:1182-8). Still another study has demonstrated that intracoronary gene transfer of fibroblast growth factor-5 increases blood flow and contractile function in an ischemic region of the heart (Giordano (1996) Nature Medicine 2:534-9). Finally, purified recombinant FGF-1 has recently been shown to induce neoangiogenesis in ischemic myocardum in human clinical trials (Schumacher, B. et al. (1998) Circulation 97:645-50). This latest study concluded that FGF treatment may be particularly suitable for patients with additional peripheral stenoses that cannot be revascularized surgically. MFGF and MFGF therapueutics are therefore likely to be effective in treating the above described cardiac disorders and conditions associated with ischemic heart disease.

The angiogenic activity of the fibroblast growth factor family is not limited to the development of cardiac blood vessels and thus the therapeutic potential of the MFGF therapeutics extends to the treatment of peripheral vascular diseases such as those arising from atherosclerotic and acute thrombotic conditions. In particular, fibroblast growth factors have been shown to be a potent angiogenic cytokine in an ischemic limb model system (Baffour, R. et al. (1992) J. Vase. Surg. 16:181-91; Takeshita, S. et al. (1994) J. Clin. Invest. 93:662-70) as well as in a rabbit cornea model system (Folkman and Klagsbrum (1987) Science 235:442-7). Furthermore, adenovirus-mediated expression of the secreted form of FGF-2 has been shown to induce cellular proliferation and angiogenesis in the ventral subcutaneous space of mice (Ueno, H. (1997) Arterioscler. Throm. Vasc. Biol. 17:2453-60). MFGF therapeutics should thus be generally useful in the treatment of peripheral vascular disease resulting from ischemic or thrombotic conditions.

Analysis of MFGF sequence has revealed that it has some homology to FGF-8. Indeed these two FGFs appear to define a distinct subfamily of the FGF family that is characterized by a distinct spacing of the two cysteines normally found in the mature form of FGFs. It is well known that homologous proteins often possess similar biological activities. FGF-8 (also known as Androgen-Induced Growth Factor or AIGF) has been shown to mediate androgen-induced cell transformation in an autocrine manner (Tanaka, et al. (1992) PNAS, USA 89:8928-8932). Interference with such an autocrine mitogenic function should block cancer cell division, thereby preventing tumor growth and eventual metastasis. Indeed it has been reported that cell division in a mouse mammary carcinoma cell line, which is stimulated by androgen through an FGF-8 dependent autocrine loop, can be markedly inhibited by antibodies to fibroblast growth factors (Yamanishi, et al. (1991) Cancer Res. 51:30063010). Furthermore, it has been shown that FGF-2 activates oncogene expression, thereby inducing the synthesis of fetal-like proteins (Parker and Schneider (1991) Annu. Rev. Physiol. 53:179-200). Thus FGF family members have been shown to be generally involved in the process of cellular transformation, therefore MFGF therapeutic agents should prove useful in the diagnosis and treatment of various cancers, particularly cancers involving the growth of steroid hormone-responsive tumors such as breast, prostate, and testicular cancers.

Indeed, FGFs 3, 4, 5, 6, 8, and 9 are all proto-oncogenes and most have been cloned from tumor lines (Slaving (1995) Cell Biol. Intl. 19:431-44). For example, FGF-3 was identified as the site of insertion of the mouse mammary tumor virus. Insertion of viral DNA within genomic FGF-3 led to gene activation and transformation of infected cells (Dickson et al. (1987) Nature 326:830-3). Furthermore, FGF-4 and FGF-5 were identified by screening neoplastic cells for the presence of genes capable of transforming 3T3 fibroblasts (Deli Bovi et al (1987) Cell 50:729-37). Most adult tumors express FGF-1 and FGF-2 as shown by immunohistochemical detection in the extracellular matrix, most notably in tumor-supporting fibroblast and endothelial cells suggesting FGF expression from host cells acting in a paracrine fashion (Ohtani et al. (1993) Lab. Invest. 68:520-7). Abnormally high levels of FGF are detectable in the serum and urine of patients with a wide range of malignancies (Nguyen et al. (1994) J. Natl. Cancer Inst. 86:356-61), and thus production of FGF by host cells appears to be a phenomenon common to many tumors and suggests that FGF plays a central role in tumor angiogenesis. Furthermore, expression of FGF may be associated with metastatic processes. In a series of 110 pigmented lesions all metastatic and primary invasive melanomas examined expressed FGF-1 and FGF expression appeared to correlate with invasion of fibroblastic reactions adjacent to the melanocyte lesions (Reed et al. (1994) Am. J. Pathol. 144:329-36). Preferred MFGF therapeutic are thus useful for treating cancer. The abovementioned observations support the use of MFGF in treating breast, prostate, testicular, and cancers of other tissues, particularly those which are known to develop steroid-responsive tumors.

In addition to their role in autocrine mitogenic stimulation of cell division, FGFs may play a role in tumor vascularization, since one of the first angiogenic factors isolated from tumors was bFGF. The ability of FGFs to stimulate the secretion of collagenase and plasminogen activator may be involoved in tumor invasion and metastasis as well as angiogenesis. Neovascularization and tumorigenicity of fibrosarcomas in transgenic mice carrying the bovine papilloma virus genome are associated with enhanced bFGF secretion (Kandel et al. (1991) Cell 66:1095-1104). Additionally, an FGF4 producing recombinant retrovirus induces tumors with a high frequency and a short latency; and amplification of the FGF-3 and FGF4 gene has been demonstrated in breast and squamous cell carcinomas and may correlate with poor prognosis. Thus the MFGF therapeutic agents may be specifically useful in preventing tumor growth by blocking neovascularization and may, furthermore, be of use in specifically preventing the spread of cancer throughout the body by the process of metastasis.

Also, many members of the FGF family bind to the same receptors (FGFR types 1, 2 and 3) and thereby elicit a second message. Based on the homology to known FGFs and similarity in signaling activity, MFGF may therefore affect any of a number of biological processes which have already been shown to be under the influence of other FGF family members. The MFGF polypeptides of the present invention may therefore also be generally employed for treating wounds (e.g. wounds due to injuries, burns, post-operative tissue repair, and ulcers) since MFGFs are potentially mitogenic to various cells of different origins, such as cardiac cells, fibroblast cells and skeletal muscle cells, and therefore, facilitate the repair or replacement of damaged or diseased tissue. The multiple activities of FGFs appear to facilitate the complex process of tissue repair in adults—i.e. a coordinated sequence of events involving platelets, leukocytes, fibroblasts, and endothelial cells. Indeed, positive effects on wound healing have been demonstrated with topical FGF treatment of certain wounds including an ischaemic rabbit ear wound model (Uhl et at. (1993) Br. J. Surg. 80:977-80) and a rat wound repair model involving random skin flaps created on the backs of rats (Ishiguro et al. (1994) Ann. Plast. Surg. 32:356-60). Furthermore, upper gastrointestinal peptic ulcers are a specific type of wound resulting from acid mediated damage to the upper gastrointestinal mucosa. Significantly, an altered form of FGF-2, which is stabilized to acid and pepsin by site-specific mutagenesis, resulted in significant acceleration of healing of duodenal ulcers in rats when administered orally (reviewed in Slavin (1995) Cell Biol. Intl. 19:43144). FGFs have been used in phase II trials in patients with gastroduodenal ulcers in the U.S.A. and Europe (Rabasseda et al. (1995) Drugs. Fut. 20:790-1). The MFGF therapeutics should therefore be generally useful in accelerating the healing of a broad range of internal and external wounds.

Neural tissue is a rich source of fibroblast growth factors and both FGF-1 and FGF-2 are widely distributed throughout the central nervous system where they act as chemotactic signals for astroglial cells (Senior et al. (1986) Biochem. Biophys. Res. Commun. 141:67-72), as proliferative signals for glial cell precursors (Engele and Bohn (1992) Dev. Biol. 152:363-72), and as stimulators of neurotrophic factor secretion by astrocytes (Yoshida, et al. (1992) J. Neurochem. 59:919-23). FGF-1 and FGF-2 have also been shown to stimulate neurite outgrowth. Furthermore, both of these FGFs, along with FGF-5, are highly expressed in the brain; while FGF-1 is highly expressed in motor neurons, primary sensory neurons, and retinal ganglion neurons suggesting that FGFs are important in neural physiology in adults (Elde, et al. (1991) Neuron 7:349-364). Significantly, fibroblast growth factors have been shown to have direct effects upon neuronal cells, e.g. in supporting neuronal survival in culture (Walicke (1988) J. Neurosci. 8:2618-27), suggesting a role of FGFs as neuroprotective agents. Indeed, systemic administration of FGF-1 at the onset of reperfusion of transient forebrain ischemia prevented severe brain injury, perhaps by alleviating damage that occurred early during reperfusion (Cuevas et al. Surg. Neurol (in press)). Furthermore FGFs have been implicated in neuronal sprouting and new synapse formation following brain infarction (Gurney et al. (1992) J. Neurosci. 12:3241-7), thereby enhancing functional recovery. These findings suggest that fibroblast growth factors have important roles in the functioning of the neuroendocrine system and that modulation of FGF bioactivities may occur in certain neurological diseases and disorders. As a member of the fibroblast growth factor family, the MFGF gene of the present invention thus provides a method of treating any of a number of such neurological diseases and disorders including e.g. Parkinson's disease, Alzheimer's disease, and cerebral palsy.

Furthermore, fibroblast growth factors play important indirect roles in neurological health through their involvement in angiogenic processes in the brain and central nervous system. Indeed fibroblast growth factors have been shown to demonstrate a biphasic pattern of gene expression following experimental cerebral ischemia, with a peak of expression that precedes and another that follows cell death (Endoh, M. et al. (1994) Mol. Brain Res. 22:76-88). It has been proposed that early expression of FGF is related to its trophic properties which support cell survival, whereas the later expression relates to its growth promoting and angiogenic properties (Cuevas (1997) Neurological Research 19:355-6). Experimental studies further suggest that angiogenesis following brain infarct is mediated by endogeneous FGFs (Cheng, et al. (1994) Stroke 25:1651-7) and that brain vessels can be manipulated by intraventricular infusion or topical application of FGFs (Lyons et al. (1991) Brain Res. 558:315-20). Thus the association between FGFs, cerebral ischemia/infarction, and angiogenesis support therapeutic administration of FGFs to stimulate angiogenic brain neovascularization which otherwise occurs naturally during brain ischemia as a self-protective process (Cuevas (1997) Neurol. Res. 19:355-6). As a member of this family of growth factors, the MFGF gene of the present invention provides a means of diagnosing and treating ischemic stroke and associated neurological diseases and disorders.

MFGF therapeutics should prove useful in treating thrombotic stroke, i.e. the traumatic loss of blood to a region of the brain due to the formation of a blood clot. Thrombotic stroke has been associated not only with altered FGF expression as has already been reviewed in the evidence implicating FGFs in protective and adaptive events following ischemic stroke, but also with the increased expression of still other trophic factors, e.g. nerve growth factor and brain-derived neurotrophic factor (Comelli, et al. (1993) Neuroscience 55:473-90). MFGF, is generally related to these neurotrophic factors, since both belong to a class of secreted ligands which can affect the growth and/or development of cells in both autocrine or paracrine mechanisms. Accordingly, the detection of altered and/or aberrant expression of MFGF may be used to predict the likelihood of thrombotic or ischemic stroke.

By analogy with the known function of other known FGF family members, MFGF and MFGF therapeutics may also be employed to stimulate chondrocyte growth thereby enhancing bone and periodontal regeneration and aiding in tissue transplants or bone grafts.

The MFGF polypeptides of the present invention may also be employed to prevent skin aging (e.g. due to sunburn by stimulating keratinocyte growth), or to prevent hair loss, since FGF family members activate hair-forming cells and promote melanocyte growth. Along the same lines, the polypeptides of the present invention may be employed to stimulate growth and differentiation of hematopoietic cells and bone marrow cells when used in combination with other cytokines.

FGFs are thought to be important agents in the maintenance of normal cellular and tissue homeostasis. The MFGF polypeptides of the present invention may therefore also be employed to maintain organs before transplantion or for supporting cell culture of primary tissues. Furthermore FGFs such as FGF4 have been shown to the stimulate the proliferation of mouse embryo limb-bud mesenchyme and thus may have a function in limb bud development (Niswander and Martin (1992) Devlopment 114: 755-68). Indeed, biological assays have identified FGF-2 activity in the developing chick limb as early as stage 18 and this expression continues in the developing limb until just prior to hatching (Niswander et al. (1993) Nature 361:68-71). Furthermore, FGFR-1 is broadly distributed in somite mesenchyme while expression of FGFR-2 occurs in regions of the somites corresponding to mesenchymal precursors of bone (Orr-Urteger (1991) Development 113:1419-34). Given the association of FGF and FGFR expression with these important embryonic developmental processes, the MFGF polypeptides of the present invention may also be employed for inducing tissue of mesodermal origin to differentiate in early embryos.

Furthermore, research on growth and differentiation inducing factors such as the FGFs has shown that they play crucial roles in the repair of damaged tissues and organs and in the regulation of the immune system and can thereby find use in agricultural applications. Specifically, members of this family have been shown to promote skeletal muscle development thereby increasing muscle mass in livestock and obviating the need for excessive use of antibiotics and hormones to improve feed conversion and weight gain in such animals. Transgenic strategies with these factors could lead to new breeds of livestock with significantly enhanced muscle mass and diminished fat content. Furthermore, pharmaceutical applications in humans include use in the development of new therapeutics for intransigent muscle-wasting conditions such as muscular dystrophy and cachexia, the muscle deterioration associated with AIDS and some cancers. The MFGF polypeptides and MFGF agonist and antagonist therapeutics of the present invention may thus have applications in both the improvement of livestock and in the treatment of muscle wasting conditions in humans.

4.9.4. Effective Dose

Toxicity and therapeutic efficacy of such compounds can be determined by standard pharmaceutical procedures in cell cultures or experimental animals, e.g., for determining The LdSO (The Dose Lethal To 50% Of The Population) And The Ed₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic induces are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

4.9.5. Formulation and Use

Pharmaceutical compositions for use in accordance with the present invention may be formulated in conventional manner using one or more physiologically acceptable carriers or excipients. Thus, the compounds and their physiologically acceptable salts and solvates may be formulated for administration by, for example, injection, inhalation or insufflation (either through the mouth or the nose) or oral, buccal, parenteral or rectal administration.

For such therapy, the compounds of the invention can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. Other suitable delivery systems include microspheres which offer the possiblity of local noninvasive delivery of drugs over an extended period of time. This technology utilizes microspheres of precapillary size which can be injected via a coronary chatheter into any selected part of the e.g. heart or other organs without causing inflammation or ischemia. The administered therapeutic is slowly released from these microspheres and taken up by surrounding tissue cells (e.g. endothelial cells).

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. in addition, detergents may be used to facilitate permeation. Transmucosal administration may be through nasal sprays or using suppositories. For topical administration, the oligomers of the invention are formulated into ointments, salves, gels, or creams as generally known in the art. A wash solution can be used locally to treat an injury or inflammation to accelerate healing.

In clinical settings, a gene delivery system for the therapeutic MFGF gene can be introduced into a patient by any of a number of methods, each of which is familiar in the art. For instance, a pharmaceutical preparation of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g., Chen et al. (1994) PNAS 91: 3054-3057). An MFGF gene, such as any one of the sequences represented in the group consisting of SEQ ID NOS 1 and 3 or a sequence homologous thereto can be delivered in a gene therapy construct by electroporation using techniques described, for example, by Dev et al. ((1994) Cancer Treat Rev 20:105-115).

The pharmaceutical preparation of the gene therapy construct or compound of the inventioncan consist essentially of the gene delivery system in an acceptable diluent, or can comprise a slow release matrix in which the gene delivery vehicle or compound is imbedded. Alternatively, where the complete gene delivery system can be produced intact from recombinant cells, e.g., retroviral vectors, the pharmaceutical preparation can comprise one or more cells which produce the gene delivery system.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration

4.10. Kits

The invention further provides kits for use in diagnostics or prognostic methods or for treating a disease or condition associated with an aberrant MFGF protein. The invention also provides kits for determining which MFGF therapeutic should be administered to a subject. The invention encompasses kits for detecting the presence of MFGF mRNA or protein in a biological sample or for determining the presence of mutations or the identity of polymorphic regions in an MFGF gene. For example, the kit can comprise a labeled compound or agent capable of detecting MFGF protein or mRNA in a biological sample; means for determining the amount of MFGF in the sample; and means for comparing the amount of MFGF in the sample with a standard. The compound or agent can be packaged in a suitable container. The kit can further comprise instructions for using the kit to detect MFGF mRNA or protein.

In one embodiment, the kit comprises a pharmaceutical composition containing an effective amount of an MFGF antagonist therapeutic and instruction for use in treating or preventing hypertension. In another embodiment, the kit comprises a pharmaceutical composition comprising an effective amount of an MFGF agonist therapeutic and instructions for use in treating insect bites. Generally, the kit comprises a pharmaceutical composition comprising an effective amount of an MFGF agonist or antagonist therapeutic and instructions for use as an analgesic. For example, the kit can comprise a pharmaceutical composition comprising an effective amount of an MFGF agonist therapeutic and instructions for use as an analgesic.

Yet other kits can be used to determine whether a subject has or is likely to develop a disease or condition associated with an aberrant MFGF activity. Such a kit can comprise, e.g., one or more nucleic acid probes capable of hybridizing specifically to at least a portion of an MFGF gene or allelic variant thereof, or mutated form thereof

4.11. Additional Uses for MFGF Proteins and Nucleic Acids

The MFGF nucleic acids of the invention can further be used in the following assays. In one embodiment, the human MFGF nucleic acid having SEQ ID NO: 1 or a portion thereof, or a nucleic acid which hybridizes thereto can be used to determine the chromosomal localization of an MFGF gene. Comparison of the chromosomal location of the MFGF gene with the location of chromosomal regions which have been shown to be associated with specific diseases or conditions, e.g., by linkage analysis (coinheritance of physically adjacent genes), can be indicative of diseases or conditions in which MFGF may play a role. A list of chromosomal regions which have been linked to specific diseases can be found, for example, in V. McKusick, Mendelian Inheritance in Man (available on line through Johns Hopkins University Welch Medical Library) and at http://www3.ncbi.nlm.nih.gov/Omim/(Online Mendelian Inheritance in Man). Furthermore, the MFGF gene can also be used as a chromosomal marker in genetic linkage studies involving genes other than MFGF.

Chromosomal localization of a gene can be performed by several methods well known in the art. For example, Southern blot hybridization or PCR mapping of somatic cell hybrids can be used for determining on which chromosome or chromosome fragment a specific gene is located. Other mapping strategies that can similarly be used to localize a gene to a chromosome or chromosomal region include in situ hybridization, prescreening with labeled flow-sorted chromosomes and preselection by hybridization to construct chromosome specific-cDNA libraries.

Furthermore, fluorescence in situ hybridization (FISH) of a nucleic acid, e.g., an MFGF nucleic acid, to a metaphase chromosomal spread is a one step method that provides a precise chromosomal location of the nucleic acid. This technique can be used with nucleic acids as short as 500 or 600 bases; however, clones larger than 2,000 bp have a higher likelihood of binding to a unique chromosomal location with sufficient signal intensity for simple detection. Such techniques are described, e.g, in Verma et al., Human Chromosomes: a Manual of Basic Techniques, Pergamon Press, New York (1988). Using such techniques, a gene can be localized to a chromosomal region containing from about 50 to about 500 genes.

If the MFGF gene is shown to be localized in a chromosomal region which cosegregates, i.e., which is associated, with a specific disease, the differences in the cDNA or genomic sequence between affected and unaffected individuals are determined. The presence of a mutation in some or all of the affected individuals but not in any normal individuals, will be indicative that the mutation is likely to be causing or contributing to the disease.

The present invention is further illustrated by the following examples which should not be construed as limiting in any way. The contents of all cited references (including literature references, issued patents, published patent applications as cited throughout this application are hereby expressly incorporated by reference. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No.: 4,683,195; Nucleic Acid Hybridization(B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### 5. EXAMPLES

5.1. Cloning and Analysis of Human MFGF

A cDNA encoding full length human MFGF was isolated. A cDNA library was first prepared from the human heart of a subject who had congestive heart failure, and a partial sequence was identified with homology to the FGF family. 3′ RACE was used to clone the 3′ end of the MFGF gene. RACE was performed using Clontech's Marathon cDNA Amplification Kit (Clontech, Palo Alto, Calif. 94303). First strand cDNA synthesis was performed using the cDNA synthesis primer supplied with the kit and 1 μg polyA⁺ RNA prepared from the heart of a 43 year old woman with an idiopathic cardiomyopathy using 100 u MMLV reverse transcriptase. Second strand cDNA synthesis was then performed using the second strand enzyme cocktail of the Clontech kit. The Marathon cDNA adaptor was ligated to the double stranded cDNA with T4 DNA ligase. A gene specific primer was designed starting at the 5′ end of the FGF-8 homolog clone. The primer had the following nucleotide sequence:

5′ CCAAGCTTCTCGAGATGTATTCAGCGCCCTCCGCCTGCACTTGCCTG 3′ (SEQ ID NO. 12). The gene specific primer and an adaptor primer were used for 3′ RACE using the Advantage Klentaq polymerase mix under the following conditions: 1 cycle at 94° C. for 2 minutes; 35 cycles of 94° C. for 30 sec., 60° C. for 45 sec., 72° C. for 30 sec.; and 1 cycle at 72° C. for 5 minutes. A 1 kb RACE product was obtained. The RACE products were run on a 1.2% agarose gel, the expected size fragments were visualized, excised and purified using the Gene Clean Gel Extraction (Bio101, Vista, Calif. 92083). The fragments were then ligated into the TA cloning vector pCR2.1 (Invitrogen, Carlsbad, Calif. 92008). A clone containing a 1.4 kb insert was sequenced and found to have the 3′ end of the gene.

The cDNA described herein encoding MFGF is 1006 bp long and has the nucleotide sequence shown in FIG. 1 and set forth in SEQ ID No. 1. A nucleic acid comprising this cDNA has been deposited at the American Type Culture Collection (12301 Parklawn Drive, Rockville, Md.) on Jan. 8, 1998 and has been assigned ATCC Designation No. 209574. This cDNA has an open reading frame from nucleotide 86 to nucleotide 706 of SEQ ID NO. 1 which is set forth in SEQ ID NO. 3 and encodes a protein of 207 amino acids having the amino acid sequence shown in FIG. 2 and set forth in SEQ ID NO. 2. The MFGF protein having SEQ ID NO. 2 contains a hydrophobic signal sequence from about amino acid 1 to amino acid 27 (SignalP (Henrik Nielsen et al., “Identification of Prokaryotic and Eukaryotic Signal Peptides and Prediction of Their Cleavage Site” 1997 Protein Engineering 10, 1-6))). Thus, the mature MFGF protein is predicted to have the amino acid sequence spanning amino acid 29 to amino acid 207 of SEQ ID NO. 2. The presence of the signal peptide indicates that MFGF is secreted and/or membrane bound. MFGF protein further comprises several functional motifs. Sequence analysis with a prosite pattern search (PDOC 00001) indicated a N-linked glycosylation site within the NQTR amino acid sequence from amino acid 39 to amino acid 42 of SEQ ID NO. 2, which is encoded by nucleotides 200 to 211 of SEQ ID NO. 1 and another N-linked glycosylation site within the NYTA amino acid sequence from amino acid 137 to amino acid 140 of SEQ ID NO. 2 which is encoded by nucleotides 494 to 505 of SEQ ID NO. 1. These N-linked glycosylation sites are characteristic of fibroblast growth factors (with the exception of bFGF).

A hydrophobicity plot analysis (data not shown) of the MFGF protein sequence shows the hydrophobic character of the putative amino-terminal secretion signal. The hydrophobic amino-terminal region of the protein stands in contrast to the relatively hydrophilic character of the remainder of the protein. These features are characteristic of secreted proteins and they thus provide further support for the relationship between MFGF and the fibroblast growth factor family of proteins. Computer sequence analysis also revealed the location of a hypothetical predicted transmembrane domain from amino acid 7 to amino acid 27 of SEQ ID NO. 2 (predicted by MEMSAT analysis), and the location of the predicted processing site at amino acid 28 of SEQ ID NO. 2 (predicted by SignalP analysis).

Several conserved features of the fibroblast growth factor family can be detected in human MFGF. These features suggest the positions of major domains of human MFGF. These domains are summarized in Table I.

The MFGF protein further comprises a pair of cysteine amino acid residues at amino acid 109 and amino acid 127 of SEQ ID NO. 2 which are encoded by nucleotide sequence from nucleotide 410 to nucleotide 412 and from nucleotide 464 to nucleotide 466 respectively of SEQ ID NO. 1. These cysteine residues occur in the predicted mature protein, but one of these cysteines is not in the position found in all other members of the FGF family, with the exception of FGF-8. While the more carboxy-terminal cysteine of both FGF-8 and MFGF is in the same position as that of the other FGF family members, the more amino-terminal cysteine is uniquely positioned only 18 residues upstream of the more carboxy-terminal cysteine and thus suggests a unique evolutionary relatedness between FGF-8 and MFGF.

Sequence Comparison

A Genbank search using BLAST (Altschul et al. (1990) J. Mol. Biol. 215: 403) of the nucleic acid and the amino acid sequences of MFGF revealed that MFGF has significant homology to ESTs, which are similar to different regions of the nucleotide sequence of MFGFs. ESTs having greater than 67% identity to regions of MFGFs are shown in Table II.

TABLE II
EST Database hits

| | | | | | | | | | |
|---|---|---|---|---|---|---|---|---|---|
| Accession # | AA656693* | Species | Mouse | Bp Covered | 416-687 | % Identity | 94 | Coding? | yes |
| Accession # | AA022949 | Species | Human | Bp Covered | 611-984 | % Identity | 87 | Coding? | yes |
| Accession # | N68951 | Species | Human | Bp Covered | 620-967 | % Identity | 87 | Coding? | yes |
| Accession # | W00630 | Species | Human | Bp Covered | 736-925 | % Identity | 88 | Coding? | no |
| Accession # | AA022987 | Species | Mouse | Bp Covered | 739-984 | % Identity | 90 | Coding? | no |
| Accession # | U55189 | Species | Chicken | Bp Covered | 191-630 | % Identity | 69 | Coding? | yes |
| Accession # | U41467 | Species | Chicken | Bp Covered | 191-630 | % Identity | 72 | Coding? | yes |
| Accession # | Z48746 | Species | Mouse | Bp Covered | 191-630 | % Identity | 67 | Coding? | yes |
| Accession # | U18673 | Species | Mouse | Bp Covered | 191-630 | % Identity | 67 | Coding? | yes |

Sequences producing high-scoring segment pairs with BLASTN included Gallus gallus fibroblast growth factor 8 mRNA (71% identical over 329 bp [nucleotides 231 to 559 of SEQ ID NO. 1] and 65% identical over 112 bp [nucleotides 120 to 231 of SEQ ID NO. 1]); Mus musculus mRNA for fibroblast growth factor 8 (67% identical over 331 bp [nucleotides 229 to 559 of SEQ ID NO. 1] and 66% identical over 115 bp [nucleotides 120 to 234 of of SEQ ID NO. 1]); and Xenopus laevis mRNA for fibroblast growth factor 8 (65% identical over 332 bp [nucleotides 228 to 559 of SEQ ID NO. 1] and 63% identical over 103 bp [nucleotides 120 to 222 of SEQ ID NO. 1]).

FIG. 3 shows an alignment of the amino acid sequence of human MFGF having SEQ ID NO. 2 and the amino acid sequence of murine MFGF having SEQ ID NO. 5 with human FGF-1 (SEQ ID NO. 7; GenBank Accession No. E03692), human FGF-2 (SEQ ID NO. 8; GenBank Accession No. E05628), mouse FGF-3 (INT-2) (SEQ ID NO. 9; GenBank Accession No. X68450), mouse FGF-13 (SEQ ID NO 10; GenBank Accession No. AF020737), and human FGF-8 (SEQ ID NO. 11; GenBank Accession No. U36223). Conserved cysteine pairs occurring in the mature protein sequence are circled and the predicted FGFR binding regions (i) and (ii) are boxed The alignment was performed using CLUSTAL W (1.7).

This amino acid sequence alignment indicates that MFGF having SEQ ID NO. 2 has the highest overall similarity to the human FGF-8 amino acid sequence and that it is about 60% identical and 75% similar to the amino acid sequence of human FGF-8. The cDNAs encoding human MFGF and FGF-8 (SEQ ID NO. 1) have an overall identity of about 68%.

Data obtained from bFGF (FGF-2) structure suggests that the binding site for heparin is a cluster of basic residues including Lys-128, Arg-129, Lys-134 and Lys-138 (Eriksson et al. 1991; Zhang et al. 1991). A similar basic sequence can be found in the sequences of both FGF-8 and MFGF as shown in Table III

TABLE III

| FGF | Amino Acids of SEQ ID NO. | Basic Sequence |
|---|---|---|
| FGF-2 | 128 to 138 (SEQ ID NO. 8) | KRTGQYKLGSK |
| FGF-8 | 154 to 164 (SEQ ID NO. 11) | TRKGRPRKGSK |
| hMFGF | 154 to 164 (SEQ ID NO. 2) | TKKGRPRKGPK |
| mMFGF | 154 to 164 (SEQ ID NO. 5) | TKKGRPRKGPK |

These functional homologies suggest that both FGF-8 and MFGF share the conserved heparin sulfate binding domain found in FGF-2. This conserved feature in MFGF has several important implications. The purification of FGF's has been greatly facilitated by their affinity for heparin and so one would expect a heparin affinity column to allow for the facile purification of MFGF. Furthermore, other FGF's are known to bind hepran sulfate proteoylycans (HGPGs), such as syndecan, present on the cell surface and in the extracellular matrix. Affinities for HSPGs vary between 2-600×10⁻⁹ M. Furthermore FGFs transduce their signals by binding to cell surface tyrosine kinase receptors (FGFRs). Interaction of many FGFs, such as bFGF (FGF-2), aFGF (FGF-1), and K-FGF (FGF4), with their cognate receptors require the presence of heparin sulfate, perhaps because of a conformational change induced by FGF binding to heparin (Yayon et al.(1991) Cell 64:841-848). Binding to heparin or heparin sulfate also protects bFGF from denaturation and proteolytic degradation, and many of the FGFs in tissues are apparently present as HSPG matrix-bound forms which can promote cell growth (Salmivirta et al. (1992) J. Biol. Chem. 267:17606-17610). Distinct classes of HSPGs may regulate, for example, neural responses to a FGF and bFGF during development (Nurcombe et al.(1993) Science 260:103-106). Thus it is likely that MFGF binding to its receptor(s) will be similarly modulated by the presence of cell surface and extracellular matrix proteoglycans.

Thus, based on the results of the BLAST analysis and the presence of characteristic functional domains, MFGF is likely to be a novel new member of the fibroblast growth factor family. Furthermore it appears that MFGF, together with FGF-8, define a novel subfamily within this group.

The BLAST analysis of GenBank with MFGF nucleic acid also indicated homologies of portions of human FGF with a number of ESTs summarized below in Table IV.

TABLE IV

| | | | % |
|---|---|---|---|
| Accession No. | Species | Nucleotides of SEQ ID No. 1 | Identity |
| AA656693* | mouse | 416-687 (coding region) | 94 |
| AA022949 | human | 611-984 (C-terminal 32aa + 3′ UTR) | 87 |
| N68951 | human | 620-967 (C-terminal 29aa + 3′ UTR) | 87 |
| W00630 | human | 736-925 (3′ UTR) | 88 |
| AA022987 | mouse | 739-984 (3′ UTR) | 90 |

*Annotation: similar to gb: Z48746 M. musculus mRNA for fibroblast growth factor

5.2 Cloning and Analysis of Murine MFGF

RACE was performed using Clontech's Marathon cDNA Amplification Kit. First strand cDNA synthesis was performed using the cDNA synthesis primer supplied with the kit and lug poly A+RNA prepared from mouse heart using 100 u MMLV reverse transcriptase. Second strand synthesis was then performed using the 2nd strand enzyme cocktail. The Marathon cDNA adaptor was ligated to the double stranded cDNA with T4 DNA ligase. For 3′ end RACE a gene specific primer was designed starting at the 5′ end of the human MFGF clone. (5′ CCAAGCTTCTCGAGATGTATTCAGCGCCCTCCGCCTGCACTTGCCTG 3′). The gene specific primer and an adaptor primer were used for 3′ RACE using the Advantage Klentaq polymerase mix (1 cycle at 94° C. for 2 minutes, 35 cycles of 94° C. for 30 sec., 60° C. for 45 sec., 72° C. for 30 sec., and 1 cycle at 72° C. for 5 minutes). A 1 kb RACE product of was obtained. The RACE products were run on a 1.2% agarose gel, the expected size fragments were visualized, excised and purified using the Gene clean Gel Extraction kit (Bio101). The fragments were then ligated into the TA cloning vector pCR2.1 (Invitrogen). A clone containing a 1.1 Kb insert was sequenced and found to have the 3 end of the gene. For 5′ end RACE the above procedure was repeated however with a gene specific primer designed starting at the 3′ end of the murine MFGF clone (5′ CTTTAGGTTCAGTTTTTGTCTTCTTTTAA 3′). On an agarose gel a 800 bp fragment was visualized which upon purification, cloning and sequencing was found to have the entire murine MFGF.

FIG. 2 shows the nucleotide sequence of a full length cDNA encoding murine MFGF including 5′ and 3′ untranslated regions and coding sequences (SEQ ID NO. 4) and the deduced amino acid sequence of the murine MFGF protein (SEQ IID NO. 5). In both FIGS. 1 and 2, the signal sequence is underlined, and the two aforementioned conserved cysteine residues which are characteristic of MFGF, FGF-8, and “FGF-13 ”, are circled. The more carboxy-terminal of these two cysteines is conserved in all known member of the fibroblast growth factor family.

5.2. Tissue Distribution of MFGF

A 398 bp EcoRI probe from the human MFGF cDNA, corresponding to nucleotides 1-398 of SEQ ID NO. 1, was labeled with ³²p using the Multiprime Labeling System from Amersham and hybridized at 10⁶ cpm/ml to Multiple Tissue Nortern blots from Clontech overnight at 65° C. in ExpressHyb Hybridization Solution from Clontech. The blots were then washed three times for 30 minutes at 65° C. in 0.1× SSC, 0.1% SDS wash buffer.

The results suggest that MFGF is expressed predominantly in the heart. In particular, the results of a human multiple tissue Northern blot reveal a single band which is evident in human heart tissue demonstrating that MFGF is expressed in cardiac muscle tissue while expression is not evident in other human organs including pancreas, kidney, liver, lung, placenta, and brain. Furthermore a human muscle multiple tissue blot has been used to demonstrate that expression appears limited to cardiac muscle as this signal is not evident in either skeletal muscle or smooth muscle tissue (prostate, stomach, bladder, small intestine, colon, and uterus).

5.3. Expression of Recombinant MFGF in COS Cells

This example describes a method for producing recombinant full length human MFGF in a mammalian expression system.

An expression construct containing a nucleic acid encoding a full length human MFGF protein, or a soluble MFGF protein which is devoid of the signal sequence can be constructed as follows. A nucleic acid encoding the full length human MFGF protein or a soluble form of MFGF protein described above is obtained by reverse transcription (RT-PCR) of mRNA extracted from human cells expressing MFGF, e.g., human cardiac tissue using PCR primers based on the sequence set forth in SEQ ID NO: 1. The PCR primers further contain appropriate restriction sites for introduction into the expression plasmid. The amplified nucleic acid is then inserted in a eukaryotic expression plasmid such as pcDNAl/Amp (In Vitrogen) containing: 1) SV40 origin of replication, 2) ampicillin resistance gens, 3) E. coli replication origin, 4) CMV promoter followed by a polylinker region, a SV40 intron and polyadenylation site. A DNA fragment encoding the full length human MFGF and a HA or myc tag fused in frame to its 3′ end is then cloned into the polylinker region of the. The HA tag corresponds to an epitope derived from the influenza hemagglutinin protein as previously described (I. Wilson, H. Niman, R. Heighten, A Cherenson, M. Connolly, and R. Lerner, 1984, Cell 37, 767). The infusion of HA tag to MFGF allows easy detection of the recombinant protein with an antibody that recognizes the HA epitope.

For expression of the recombinant MFGF, COS cells are transfected with the expression vector by DEAE-DEXTRAN method. (J. Sambrook, E. Fritsch, T. Maniatis, Molecular Cloning: A Laboratory Manual, Cold Spring Laboratory Press, (1989)). The expression of the MFGF-HA protein can be detected by radiolabelling and immunoprecipitation with an anti-HA antibody. (E. Harlow, D. Lane, Antibodies: A Laboratory Manual, Cold Spring Harbor Laboratory Press, (1988)). For this, transfected cells are labeled with ³⁵S-cysteine two days post transfection. The cells, or alternatively the culture media (e.g., for the soluble MFGF) is then collected and the MFGF protein immunoprecipitated with an HA specific monoclonal antibody. To determine whether full length MFGF is a membrane protein, and/or a secreted protein, the cells transfected with a vector encoding the full length MFGF protein can be lysed with detergent (RIPA buffer (150 mM NaCI 1% NP40, 0.1% SDS, 1% NP40, 0.5% DOC, 50 mM Tris, pH 7.5). (Wilson, I. et al., Id. 37:767 (1984)). Proteins precipitated can then be analyzed on SDS-PAGE gel. Thus, the presence of MFGF in the cell will be indicative that the full length MFGF can be membrane bound and the presence of MFGF in the supernatant will be indicative that the protein can also be in a soluble form, whether produced as a secreted protein or released by leakage from the cell.

Equivalents

Those skilled in the art will recognize, or be able to ascertain using no more than routine experimentation, many equivalents of the specific embodiments of the invention described herein. Such equivalents are intended to be encompassed by the following claims.

1. An isolated nucleic acid comprising a nucleotide sequence which is at least about 70% identical to the entire nucleotide sequence set forth in SEQ ID NO: 1, 3, 4 or 6 or complement thereof.

3. The isolated nucleic acid of claim 1, which is mammalian.

4. The isolated nucleic acid of claim 3, which is from a human.

5. The isolated nucleic acid of claim 4, which is comprised of the nucleic acid having ATCC Designation No. 209574 or 209648.

6. An isolated nucleic acid comprising at least about 15 consecutive nucleotides having a nucleotide sequence which is at least about 75% identical to a nucleotide sequence set forth in SEQ ID NOS: 1 or 4 or a complement thereof, with the proviso that the nucleic acid is not selected from the group consisting of the EST sequences having GenBank Accession Nos. AA656693, AA022949, N68951, W00630, and AA022987.

7. The isolated nucleic acid of claim 6, which is located in a region selected from the group consisting of: nucleotides 86-169; nucleotides 170-706; nucleotides 545-577; nucleotides 182-199; nucleotides 410-466; and nucleotides 539-568 of SEQ ID NO: 1.

8. The isolated nucleic acid of claim 6, further comprising a label.

9. An isolated nucleic acid comprising at least about 15 consecutive nucleotides, which nucleic acid hybridizes under high stringency conditions to a nucleotide sequence set forth in SEQ ID NOS: 1, 3, 4 or 6 or a complement thereof or to the nucleic acid having ATCC Designation No. 209574 or 209648, provided that the nucleic acid is not a member selected from the group consisting of the EST sequences having GenBank Accession Nos. AA656693, AA022949, N68951, W00630, and AA022987.

10. The isolated nucleic acid of claim 9, which is located in a region selected from the group consisting of: nucleotides 86-169; nucleotides 170-706; nucleotides 545-577; nucleotides 182-199; nucleotides 410-466; and nucleotides 539-568 of SEQ ID NO: 1.

11. An isolated nucleic acid comprising a nucleotide sequence encoding a polypeptide having an amino acid identity of at least about 70% with the entire amino acid sequence of an MFGF polypeptide set forth in SEQ ID NO: 2 or SEQ ID NO: 5.

12. The isolated nucleic acid of claim 11, wherein the polypeptide is a mammalian polypeptide.

13. The isolated nucleic acid of claim 12, wherein the polypeptide is a human polypeptide.

14. The isolated nucleic acid of claim 11, wherein the polypeptide is a soluble polypeptide.

15. The isolated nucleic acid of claim 14, wherein the polypeptide is a fusion polypeptide.

16. A vector comprising a nucleic acid of claim 1.

17. A host cell comprising the vector of claim 16.

18. An isolated polypeptide comprising an amino acid sequence having an amino acid identity of at least about 70% with the entire amino acid sequence set forth in SEQ ID NO: 2 or 5.

19. The isolated polypeptide of claim 18, which is a mammalian polypeptide.

20. The isolated polypeptide of claim 19, wherein the polypeptide is a human polypeptide.

21. The isolated polypeptide of claim 20, which is encoded by the nucleic acid having ATCC Designation No. 209574 or ATCC Designation No. 209648.

22. The isolated polypeptide of claim 19, which has the amino acid sequence set forth in SEQ ID NO: 2 or SEQ ID NO: 5.

23. A method for producing a polypeptide of claim 18, comprising incubating a host cell comprising a nucleic acid encoding the polypeptide of claim 18 operably linked to a regulatory element, thereby resulting in expression of the polypeptide.

24. The method of claim 23, wherein the host cell is in vivo.

25. A method for identifying a compound that modulates a MFGF bioactivity, comprising the steps of:
(a) contacting an appropriate amount of the compound with a cell or cellular extract, which expresses a MFGF gene; and
(b) determining the resulting MFGF bioactivity, wherein an increase or decrease in the MFGF bioactivity in the presence of the compound as compared to the bioactivity in the absence of the compound indicates that the compound is a modulator of a MFGF bioactivity.

26. The method of claim 25, wherein the compound is an agonist of a MFGF bioactivity.

27. The method of claim 25, wherein the compound is an antagonist of a MFGF bioactivity.

28. A method for identifying a compound that modulates a MFGF bioactivity comprising the steps of:
(i) combining an MFGF protein, an MFGF binding partner, and a test compound under conditions wherein, but for the test compound, the MFGF protein and MFGF binding partner are able to interact; and
(ii) detecting the formation of an MFGF protein/MFGF binding partner complex, such that a difference in the formation of an MFGF protein/MFGF binding partner complex in the presence of a test compound relative to the absence of the test compound is indicative that the test compound is an MFGF therapeutic.

29. The method of claim 25, wherein the compound is a member selected from the group consisting of a polypeptide, a nucleic acid, a peptidomimetic, and a small molecule.

30. The method of claim 29, wherein the small molecule is a steroid.

31. The method of claim 29, wherein the nucleic acid is a member selected from the group consisting of a gene replacement, an antisense, a ribozyme, and a triplex nucleic acid.

32. A method for treating or preventing a disease, which is caused by or contributed to by an aberrant MFGF activity in a subject, comprising administering to the subject an effective amount of an MFGF therapeutic.

33. A method of claim 32, where the disease is selected from the group consisting of: a cardiovascular disease; a neurodegenerative disease and a cancer.

34. The method of claim 32, wherein the cancer is associated with the growth of a steroid responsive tumor.

35. The method of claim 33, wherein the cardiovascular disease is a member selected from the group consisting of: hypertension, hypotension, cardiomyocyte hypertrophy, congestive heart failure or myocardial infarction.

36. A method for determining whether a subject has or is at risk of developing a disease or condition which is caused or contributed to by an aberrant MFGF activity, comprising measuring in the subject or in a sample obtained from the subject at least one MFGF activity, wherein a difference in the MFGF activity relative to the MFGF activity in a normal subject indicates that the subject is at risk of developing a disease caused by or contributed to by an aberrant MFGF activity.

37. The method of claim 36, wherein an MFGF activity is determined by measuring the protein level of an MFGF protein.

38. The method of claim 37, comprising determining whether the MFGF gene of the subject comprises a genetic alteration.

39. The method of claim 38, wherein determining whether a subject's MFGF gene comprises a genetic alteration, further comprises the steps of:
(i) contacting a nucleic acid comprising at least a portion of the MFGF gene from a subject with at least one nucleic acid probe capable of hybridizing with a wild-type MFGF gene; and
(ii) detecting the formation of a hybrid between the portion of the MFGF gene from the subject and the at least one nucleic acid probe, wherein the absence of hybrid formation indicates that the subject's MFGF gene contains a genetic alteration.

40. A method for determining whether a subject has or is at risk of developing a disease or condition, which is caused by or contributed to by an aberrant MFGF activity comprising measuring in the subject or in a sample obtained from the subject at least one MFGF activity, wherein a difference in the MFGF activity relative to the MFGF activity in a normal subject indicates that the subject has or is at risk of developing the disease or condition.

41. The method of claim 40, comprising determining whether the MFGF gene of the subject comprises a genetic alteration.

42. The method of claim 40, wherein the disease or condition is selected from the group consisting of a cardiovascular disease, a cancer and a neurodegenerative disease.

43. The method of claim 42, wherein the cardiovascular disease or condition is selected from the group consisting of hypertension, hypotension, cardiomyocyte hypertrophy, congestive heart failure or myocardial infarction.

44. A method for establishing an MFGF genetic population profile in a specific population of individuals, comprising determining the MFGF genetic profile of the individuals in the population and establishing a relationship between MFGF genetic profiles and specific characteristics of the individuals.

45. The method of claim 44, wherein the specific characteristics of the individual include the response of an individual to an MFGF therapeutic.

46. A method for selecting the appropriate MFGF therapeutic to administer to an individual having a disease or condition caused by or contributed to by a deficient MFGF gene and/or protein, comprising determining the MFGF genetic profile of an individual and comparing the individual's MFGF genetic profile to an MFGF genetic population profile, to thereby select the appropriate MFGF therapeutic for administration to the individual.

47. The method of claim 46, wherein determining the MFGF genetic profile of an individual comprises determining the identity of a single nucleotide polymorphism.

48. A kit for determining whether a subject has or is likely to develop a disease or condition, which is caused by or contributed to by an aberrant MFGF activity, comprising a probe or primer capable of hybridizing to an MFGF nucleic acid and instructions for use.
